(19) United States
(12) Patent Application Publication
(10) Pub. No.: US 2025/0269037 A1
(43) Pub. Date: Aug. 28, 2025

(54) COMPOUNDS, COMPLEXES, AND METHODS FOR THEIR PREPARATION AND OF THEIR USE

(71) Applicant: Revolution Medicines, Inc., Redwood City, CA (US)

(72) Inventors: G. Leslie BURNETT, Redwood City, CA (US); Anne V. EDWARDS, San Mateo, CA (US); Adrian L. GILL, Atherton, CA (US); Micah James GLIEDT, Fremont, CA (US); John E. KNOX, Emerald Hills, CA (US); Elena S. KOLTUN, Foster City, CA (US); James AGGEN, Burlingame, CA (US); Andreas BUCKL, Basel (CH); Jennifer PITZEN, Oakland, CA (US); Christopher SEMKO, Fremont, CA (US)

(21) Appl. No.: 19/200,842

(22) Filed: May 7, 2025

Related U.S. Application Data
(63) Continuation of application No. PCT/US2023/037057, filed on Nov. 9, 2023.
(60) Provisional application No. 63/424,079, filed on Nov. 9, 2022.

(51) Int. Cl. A61K 47/54; C07D 203/08; C07D 203/24; C07D 405/04; C07F 1/02

(52) U.S. Cl. CPC A61K 47/545; C07D 203/08; C07D 203/24; C07D 405/04; C07F 1/02

(57) ABSTRACT

The invention features compounds containing an aziridine moiety and methods of synthesizing the same. The compounds may be bound to a monovalent organic moiety and may be used to bind to a target (e.g., a target protein), for example, by cross-linking the target. In some embodiments, the monovalent organic moiety is capable of binding to a presenter protein. Also disclosed are complexes containing the compounds (e.g., presenter protein/compound complexes, compound/target protein complexes, or tri-complexes). Methods of forming the complexes and methods of using the compounds and complexes to modulate biological processes are also disclosed.

### BACKGROUND

The vast majority of small molecule drugs act by binding a functionally important pocket on a target protein, thereby modulating the activity of that protein. For example, cholesterol-lowering drugs known as statins bind the enzyme active site of HMG-CoA reductase, thus preventing the enzyme from engaging with its substrates. The fact that many such drug/target interacting pairs are known may have misled some into believing that a small molecule modulator could be discovered for most, if not all, proteins provided a reasonable amount of time, effort, and resources. This is far from the case. Current estimates are that only about 10% of all human proteins are targetable by small molecules. Bojadzic and Buchwald, Curr Top Med Chem 18: 674-699 (2019). The other 90% are currently considered refractory or intractable toward above-mentioned small molecule drug discovery. Such targets are commonly referred to as “undruggable.” These undruggable targets include a vast and largely untapped reservoir of medically important human proteins. Thus, there exists a great deal of interest in discovering new molecular modalities capable of modulating the function of such undruggable targets. Particularly, there is a need for new compounds capable of forming complexes with a target of interest (e.g., a target protein).

### SUMMARY

In general, the present disclosure features compounds capable of forming complexes and/or crosslinking to a target (e.g., a target protein). Also disclosed are synthetic intermediates used in the preparation of such compounds, complexes formed from reaction with the compounds, methods of synthesizing the compounds and complexes, and methods of using the compounds and complexes.

In a first aspect, the disclosure provides a compound having the structure of Formula I:

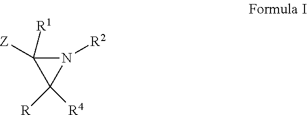

or a pharmaceutically acceptable salt thereof; wherein
Z is

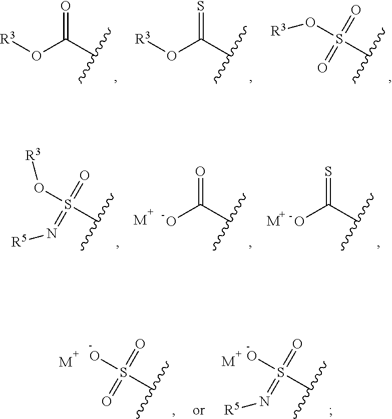

M⁺ is a cation;
R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl or —Si(R^{1a})₃; or
R and R¹ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R and R² combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and R¹ and R⁴ are each, independently, hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a}); or
R and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R² combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and each of R and R⁴ is, independently, hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R² and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and each of R¹ and R is, independently, hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
wherein at least one of R and R⁴ is not hydrogen;
R³ is hydrogen, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₃-C₁₀ cycloalkyl, optionally substituted C₃-C₁₀ cycloalkenyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl;
each R^{1a}, R^{2a}, R^{2c}, R^{2d}, and R^{2e} is, independently, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl;
R^{2b} is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁵ is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl,
and wherein each hydrogen is independently, optionally, isotopically enriched for deuterium.

In some embodiments, the compound, or pharmaceutically acceptable salt thereof, has the structure of Formula Ia:

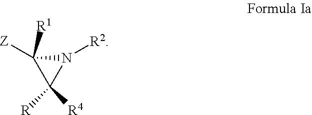

In some embodiments, the compound, or pharmaceutically acceptable salt thereof, has the structure of Formula Ib:

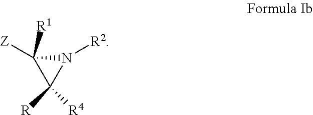

In some embodiments, the compound, or pharmaceutically acceptable salt thereof, has the structure of Formula Ic:

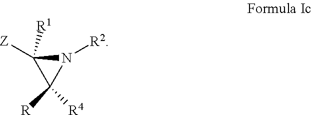

In some embodiments, the compound, or pharmaceutically acceptable salt thereof, has the structure of Formula Id:

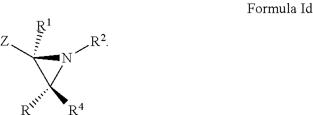

In some embodiments, R³ is optionally substituted C₁-C₆ alkyl, optionally substituted 3- to 10-membered heterocycloalkyl, optionally substituted C₆-C₁₀ aryl, or optionally substituted 5- to 10-membered heteroaryl. In some embodiments, R³ is optionally substituted C₁-C₆ alkyl. In some embodiments, R³ is methyl, ethyl, or benzyl.

In some embodiments, M⁺ is Li⁺.

In some embodiments, R³ is:

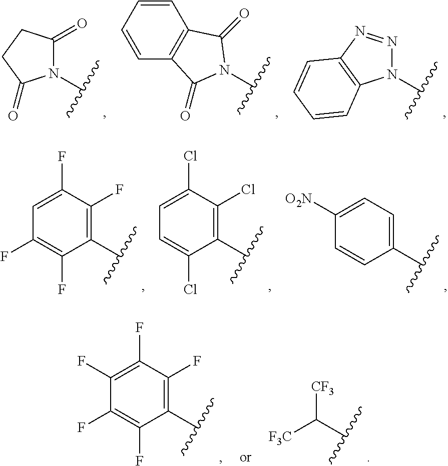

In some embodiments, Z is

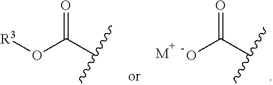

In another aspect, the disclosure provides a compound having the structure of Formula II:

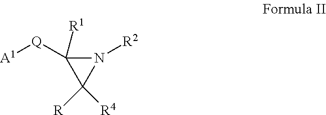

or a pharmaceutically acceptable salt thereof,
wherein A¹ is a monovalent organic moiety;
Q is

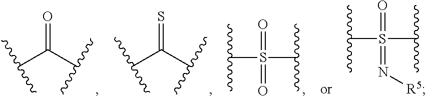

R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl or —Si(R^{1a})₃;
R and R¹ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R and R² combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and R¹ and R⁴ are each, independently, hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a}); or
R and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R² combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and each of R and R⁴ is, independently, hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R² and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and each of R¹ and R is, independently, hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
wherein at least one of R and R⁴ is not hydrogen;
each R^{1a}, R^{2a}, R^{2c}, R^{2d}, and R^{2e} is, independently, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl;
R^{2b} is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁵ is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl,
and wherein each hydrogen is independently, optionally, isotopically enriched for deuterium.

In some embodiments, the compound of Formula II, or a pharmaceutically acceptable salt thereof, has the structure of Formula IIa:

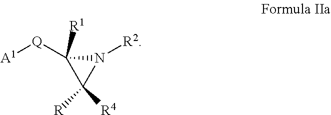

In some embodiments, the compound of Formula II, or a pharmaceutically acceptable salt thereof, has the structure of Formula IIb:

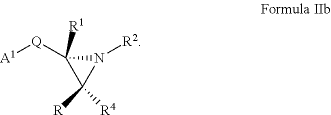

In some embodiments, the compound of Formula II, or a pharmaceutically acceptable salt thereof, has the structure of Formula IIc:

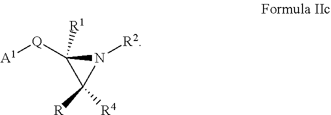

In some embodiments, the compound of Formula II, or a pharmaceutically acceptable salt thereof, has the structure of Formula IId:

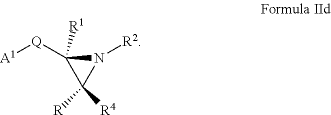

In some embodiments, Q is

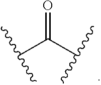 [SMILES: *C(*)=O]

In a further aspect, the disclosure provides a compound/target protein complex, or a pharmaceutically acceptable salt thereof, having the structure of Formula III:

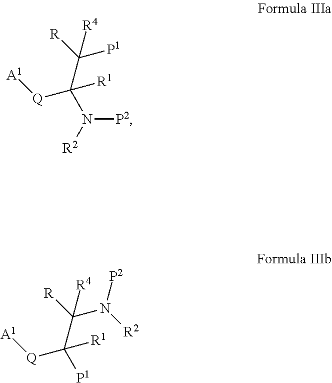

wherein A¹ is a monovalent organic moiety;
Q is

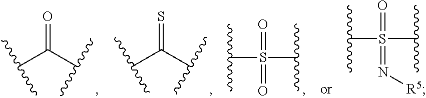

P¹ is A², and P² is hydrogen; or P¹ is hydroxyl, and P² is A²;
A² is the target protein;
R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl or —Si(R^{1a})₃;
or R and R¹ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R and R² combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and R¹ and R⁴ are each, independently, hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a}); or
R and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R² combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and each of R and R⁴ is, independently, hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R² and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and each of R¹ and R is, independently, hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
wherein at least one of R and R⁴ is not hydrogen;
each R^{1a}, R^{2a}, R^{2c}, R^{2d} and R^{2e} is, independently, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl;
R^{2b} is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted 5- to 10-membered heterocycloalkenyl, optionally substituted 8- to 10-membered heterocycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁵ is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl,
and wherein each hydrogen is independently, optionally, isotopically enriched for deuterium.

In some embodiments, the compound/target protein complex has the structure of Formula IIIa-1:

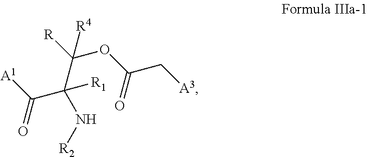

wherein A³ is the rest of the target protein.

In some embodiments, the compound/target protein complex has the structure of Formula IIIa-2:

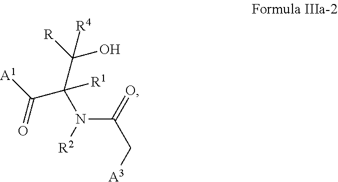

wherein A³ is the rest of the target protein.

In some embodiments, the compound/target protein complex has the structure of Formula IIIa-3:

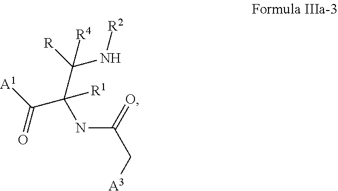

wherein A³ is the rest of the target protein.

In some embodiments, the compound/target protein complex has the structure of Formula IIIa-4:

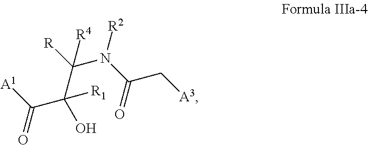

wherein A³ is the rest of the target protein.

In some embodiments, the compound/target protein complex has the structure of/target protein complex of claim 16, wherein the compound/target protein complex has the structure of Formula IIIb-1:

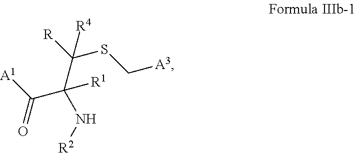

wherein A³ is the rest of the target protein.

In some embodiments, the compound/target protein complex has the structure of Formula IIIb-2:

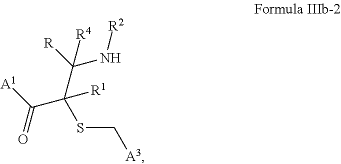

wherein A³ is the rest of the target protein.

In some embodiments, the compound/target protein complex has the structure of Formula IIIc-1:

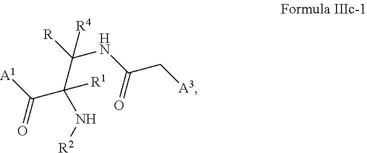

wherein A³ is the rest of the target protein.

In some embodiments, the compound/target protein complex has the structure of Formula IIIc-2:

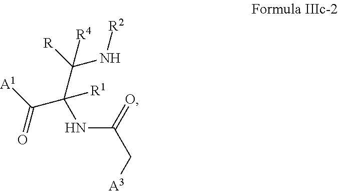

wherein A³ is the rest of the target protein.

In some embodiments, the compound/target protein complex has the structure of Formula IIIc-3:

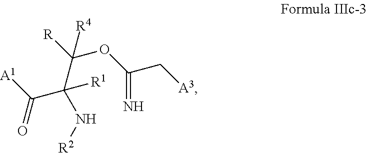

wherein A³ is the rest of the target protein.

In some embodiments, the compound/target protein complex has the structure of Formula IIIc-4:

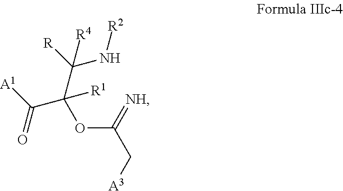

wherein A³ is the rest of the target protein.

In some embodiments, the compound/target protein complex has the structure of Formula IIId-1:

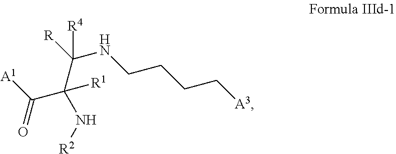

wherein A³ is the rest of the target protein.

In some embodiments, the compound/target protein complex has the structure of Formula IIId-2:

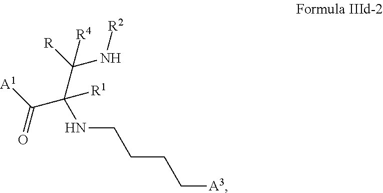

wherein A³ is the rest of the target protein.

In some embodiments, the compound/target protein complex has the structure of Formula IIIe-1:

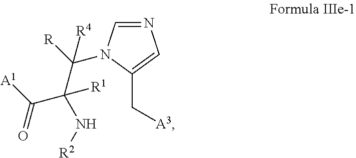

wherein A³ is the rest of the target protein.

In some embodiments, the compound/target protein complex has the structure of Formula IIIe-2:

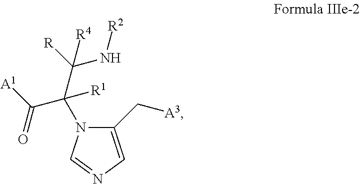

wherein A³ is the rest of the target protein.

In some embodiments of any of the compounds or compound/target protein complexes described herein, R is optionally substituted C₃-C₁₀ cycloalkyl, optionally substituted C₆-C₁₀ aryl, optionally substituted C₂-C₆ alkenyl, or optionally substituted C₂-C₆ alkynyl. In some embodiments, R is optionally substituted C₃-C₁₀ cycloalkyl. In some embodiments, R is optionally substituted cyclopropyl. In some embodiments, R is

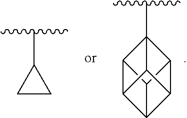

In some embodiments, R is

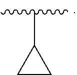 [SMILES: *C1CC1]

In some embodiments, R is optionally substituted C₂-C₆ alkenyl, or optionally substituted C₂-C₆ alkynyl. In some embodiments, R is

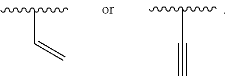

In some embodiments, R is a carbocation-stabilizing electron-donating group, that is, R is a substituent capable of stabilizing a positive or partial positive charge on the carbon to which it is attached.

In some embodiments of any of the compounds or compound/target protein complexes described herein, R¹ is hydrogen.

In some embodiments of any of the compounds or compound/target protein complexes described herein, R² is hydrogen, optionally substituted C₁-C₆ alkyl, optionally substituted C₁-C₆ heteroalkyl, optionally substituted C₃-C₁₀ cycloalkyl, or optionally substituted 3- to 10-membered heterocycloalkyl. In some embodiments, R² is optionally substituted C₁-C₆ alkyl, optionally substituted C₁-C₆ heteroalkyl, optionally substituted C₃-C₁₀ cycloalkyl, or optionally substituted 3- to 10-membered heterocycloalkyl. In some embodiments, R² is optionally substituted C₁-C₆ alkyl. In some embodiments, R² is optionally substituted C₃-C₁₀ cycloalkyl. In some embodiments, R² is optionally substituted 3- to 10-membered heterocycloalkyl. In some embodiments, R² is:

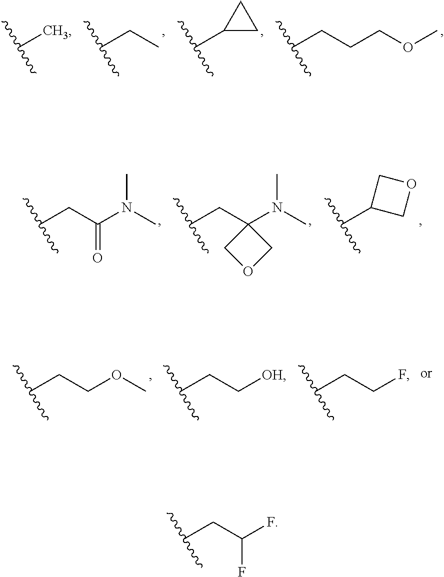

In some embodiments, R² is:

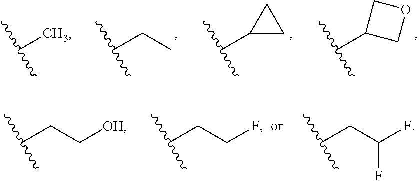

In some embodiments, R² is methyl.

In some embodiments of any of the compounds or compound/target protein complexes described herein, R⁴ is hydrogen.

In some embodiments of the compounds or compound/target protein complexes described herein, A¹ is or comprises a protein. In some embodiments, A¹ is or comprises a nucleic acid. In some embodiments, A¹ is or comprises a small molecule. In some embodiments, A¹ is or comprises a macrocyclic small molecule. In some embodiments A¹ is or compromises a degrader.

In some embodiments, A¹ has the structure of Formula IV:

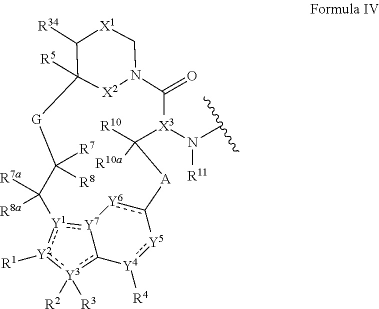

wherein the dotted lines represent zero, one, two, three, or four non-adjacent double bonds;
A is —N(H or CH₃)C(O)—(CH₂)— where the amino nitrogen is bound to the carbon atom of —CH(R¹⁰)—, optionally substituted C₂-C₄ alkylene, optionally substituted C₁-C₄ heteroalkylene, or optionally substituted C₂-C₄ alkenylene, optionally substituted 3- to 6-membered cycloalkylene, optionally substituted 3- to 6-membered heterocycloalkylene, optionally substituted 6-membered arylene, or optionally substituted 5- to 10-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂ or N;
Y⁶ is C(O), CH, CH₂, or N;
R¹ is cyano, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6 to 10-membered aryl, or optionally substituted 5- to 10-membered heteroaryl, or
R¹ and R² combine with the atoms to which they are attached to form an optionally substituted 3- to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl; R³ is absent, or
R² and R³ combine with the atom to which they are attached to form an optionally substituted 3- to 8-membered cycloalkyl or optionally substituted 3- to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or R⁶ and R⁷ combine with the carbon atoms to which they are attached to form an optionally substituted 3- to 6-membered cycloalkyl or optionally substituted 3- to 7-membered heterocycloalkyl; R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6 to 10-membered aryl, or R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3- to 6-membered cycloalkyl, or optionally substituted 3- to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6 to 10-membered aryl, or R^{7′} and R^{8′} combine with the carbon atom to which they are attached to form optionally substituted 3- to 6-membered cycloalkyl or optionally substituted 3- to 7-membered heterocycloalkyl;
R⁹ is hydrogen, F, optionally substituted C₁-C₆ alkyl, optionally substituted C₁-C₆ heteroalkyl, optionally substituted 3- to 6-membered cycloalkyl, or optionally substituted 3- to 7-membered heterocycloalkyl, or
R⁹ and L combine with the atoms to which they are attached to form an optionally substituted 3- to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R³⁴ is hydrogen or C₁-C₃ alkyl, and wherein each hydrogen is independently, optionally, isotopically enriched for deuterium.

In some embodiments, A¹ has the structure of Formula V:

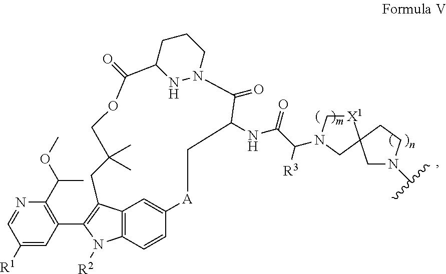

wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
X¹ is CH₂ or O;
m is 1 or 2;
n is 0 or 1;
R¹ is hydrogen or optionally substituted 3- to 10-membered heterocycloalkyl;
R² is optionally substituted C₁-C₆ alkyl; and
R³ is optionally substituted C₁-C₆ alkyl or optionally substituted 3- to 6-membered cycloalkyl, and wherein each hydrogen is independently, optionally, isotopically enriched for deuterium.

In some embodiments, A¹ has the structure of Formula VI:

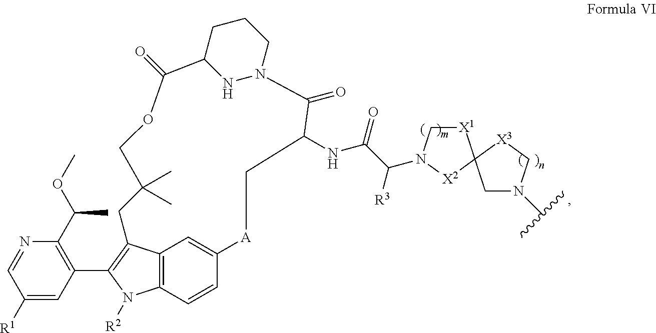

wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene; X¹, X², and X³ are each independently selected from CH₂, CHF, CF₂, C═O, or O;
m is 1 or 2;
n is 0 or 1;
R¹ is hydrogen, optionally substituted C₁-C₆ heteroalkyl, or optionally substituted 3- to 10-membered heterocycloalkyl;
R² is optionally substituted C₁-C₆ alkyl; and
R³ is optionally substituted C₁-C₆ alkyl, optionally substituted C₁-C₆ heteroalkyl, optionally substituted 3- to 6-membered cycloalkyl, or optionally substituted heterocycloalkyl,
and wherein each hydrogen is independently, optionally, isotopically enriched for deuterium.

In some embodiments, A¹ has the structure of any one of Formula VII, VIII, and IX:

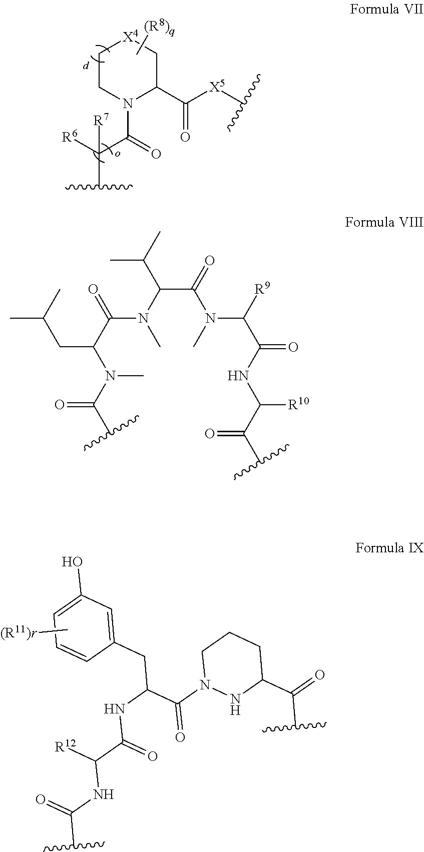

wherein o, and p are independently 0, 1, or 2;
q is an integer between 0 and 7;
r is an integer between 0 and 4;
X⁴ and X⁵ are each, independently, absent, CH₂, O, S, SO, SO₂, or NR¹¹;
R⁶ and R⁷ are, independently, hydrogen, hydroxyl, optionally substituted amino, halogen, thiol, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted C₃-C₁₀ carbocyclyl, optionally substituted C₆-C₁₀ aryl, optionally substituted C₆-C₁₀ aryl C₁-C₆ alkyl, optionally substituted C₂-C₉ heteroaryl, optionally substituted C₂-C₉ heteroaryl C₁-C₆ alkyl, optionally substituted C₂-C₉ heterocyclyl, optionally substituted C₂-C₉ heterocyclyl C₁-C₆ alkyl, or R⁶ and R⁷ combine with the carbon atom to which they are bound to form C═O;
each R⁸ is, independently, hydroxyl, optionally substituted amino, halogen, thiol, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted C₃-C₁₀ carbocyclyl, optionally substituted C₆-C₁₀ aryl, optionally substituted C₆-C₁₀ aryl C₁-C₆ alkyl, optionally substituted C₂-C₉ heteroaryl, optionally substituted C₂-C₉ heteroaryl C₁-C₆ alkyl, optionally substituted C₂-C₉ heterocyclyl, or optionally substituted C₂-C₉ heterocyclyl C₁-C₆ alkyl, or two R⁸ combine to form an optionally substituted C₃-C₁₀ carbocyclyl, optionally substituted C₆-C₁₀ aryl, or optionally substituted C₂-C₉ heteroaryl;
R⁹ is optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted C₃-C₁₀ carbocyclyl, optionally substituted C₆-C₁₀ aryl, optionally substituted C₆-C₁₀ aryl C₁-C₆ alkyl, optionally substituted C₂-C₉ heteroaryl, optionally substituted C₂-C₉ heteroaryl C₁-C₆ alkyl, optionally substituted C₂-C₉ heterocyclyl, or optionally substituted C₂-C₉ heterocyclyl C₁-C₆ alkyl;
R¹⁰ is optionally substituted C₁-C₆ alkyl;
each R¹¹ is, independently, hydroxyl, cyano, optionally substituted amino, halogen, thiol, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted C₃-C₁₀ carbocyclyl, optionally substituted C₆-C₁₀ aryl, optionally substituted C₆-C₁₀ aryl C₁-C₆ alkyl, optionally substituted C₂-C₉ heteroaryl, optionally substituted C₂-C₉ heteroaryl C₁-C₆ alkyl, optionally substituted C₂-C₉ heterocyclyl, or optionally substituted C₂-C₉ heterocyclyl C₁-C₆ alkyl; and
R¹² and R¹³ are each, independently, hydrogen, optionally substituted C₁-C₆ alkyl, optionally substituted C₂-C₆ alkenyl, optionally substituted C₂-C₆ alkynyl, optionally substituted aryl, C₃-C₇ carbocyclyl, optionally substituted C₆-C₁₀ aryl C₁-C₆ alkyl, and optionally substituted C₃-C₇ carbocyclyl C₁-C₆ alkyl.

In yet another aspect, the disclosure provides a tri-complex. The tri-complex includes a presenter protein, a compound described herein, and a target protein.

In some embodiments of the compound/target protein complexes or the tri-complexes described herein, the target protein is a GTPase, GTPase activating protein, Guanine nucleotide-exchange factor, a heat shock protein, an ion channel, a coiled-coil protein, a kinase, a phosphatase, a ubiquitin ligase, a transcription factor, a chromatin modifier/remodeler, a protease, or a protein with classical protein-protein interaction domains and motifs.

In some embodiments, the target protein is a GTPase selected from DIRAS1, DIRAS2, DIRAS3, ERAS, GEM, HRAS, KRAS, MRAS, NKIRAS1, NKIRAS2, NRAS, RALA, RALB, RAP1A, RAP1B, RAP2A, RAP2B, RAP2C, RASD1, RASD2, RASL10A, RASL10B, RASL11A, RASL11B, RASL12, REM1, REM2, RERG, RERGL, RRAD, RRAS, RRAS2, RHOA, RHOB, RHOBTB1, RHOBTB2, RHOBTB3, RHOC, RHOD, RHOF, RHOG, RHOH, RHOJ, RHOQ, RHOU, RHOV, RND1, RND2, RND3, RAC1, RAC2, RAC3, CDC42, RAB1A, RAB1B, RAB2, RAB3A, RAB3B, RAB3C, RAB3D, RAB4A, RAB4B, RABSA, RAB5B, RAB5C, RAB6A, RAB6B, RAB6C, RAB7A, RAB7B, RAB7L1, RAB8A, RAB8B, RAB9, RAB9B, RABL2A, RABL2B, RABL4, RAB10, RAB11A, RAB11B, RAB12, RAB13, RAB14, RAB15, RAB17, RAB18, RAB19, RAB20, RAB21, RAB22A, RAB23, RAB24, RAB25, RAB26, RAB27A, RAB27B, RAB28, RAB2B, RAB30, RAB31, RAB32, RAB33A, RAB33B, RAB34, RAB35, RAB36, RAB37, RAB38, RAB39, RAB39B, RAB40A, RAB40AL, RAB40B, RAB40C, RAB41, RAB42, RAB43, RAP1A, RAP1B, RAP2A, RAP2B, RAP2C, ARF1, ARF3, ARF4, ARF5, ARF6, ARL1, ARL2, ARL3, ARL4, ARL5, ARL5C, ARL6, ARL7, ARL8, ARL9, ARL10A, ARL10B, ARL10C, ARL11, ARL13A, ARL13B, ARL14, ARL15, ARL16, ARL17, TRIM23, ARL4D, ARFRP1, ARL13B, RAN, RHEB, RHEBL1, RRAD, GEM, REM, REM2, RIT1, RIT2, RHOT1, and RHOT2. In some embodiments, the target protein is a member of the RAS family. In some embodiments, the target protein is HRAS, KRAS, or NRAS. In some embodiments, the target protein is mutated HRAS, mutated KRAS, or mutated NRAS. In some embodiments, the target protein is not RAS (e.g., not HRAS, not KRAS, or not NRAS, such as not a mutated HRAS, not a mutated KRAS, or not a mutated NRAS). In some embodiments, the target protein is KRAS. In some embodiments, the target protein is a GTPase activating factor selected from NF1, IQGAP1, PLEXIN-B1, RASAL1, RASAL2, ARHGAP5, ARHGAP8, ARHGAP12, ARHGAP22, ARHGAP25, BCR, DLC1, DLC2, DLC3, GRAF, RALBP1, RAP1GAP, SIPA1, TSC2, AGAP2, ASAP1, and ASAP3.

In some embodiments, target protein is a Guanine nucleotide-exchange factor selected from CNRASGEF, RASGEF1A, RASGRF2, RASGRP1, RASGRP4, SOS1, RALGDS, RGL1, RGL2, RGR, ARHGEF10, ASEF/ARHGEF4, ASEF2, DBS, ECT2, GEF-H1, LARG, NET1, OBSCURIN, P-REX1, P-REX2, PDZ-RHOGEF, TEM4, TIAM1, TRIO, VAV1, VAV2, VAV3, DOCK1, DOCK2, DOCK3, DOCK4, DOCK8, DOCK10, C₃G, BIG2/ARFGEF2, EFA6, FBX8, and GEP100.

In some embodiments, the target protein is a protein with a protein-protein interaction domain selected from ARM, BAR, BEACH, BH, BIR, BRCT, BROMO, BTB, C₁, C₂, CARD, CC, CALM, CH, CHROMO, CUE, DEATH, DED, DEP, DH, EF-hand, EH, ENTH, EVH1, F-box, FERM, FF, FH2, FHA, FYVE, GAT, GEL, GLUE, GRAM, GRIP, GYF, HEAT, HECT, IQ, LRR, MBT, MH1, MH2, MIU, NZF, PAS, PB1, PDZ, PH, POLO-Box, PTB, PUF, PWWP, PX, RGS, RING, SAM, SC, SH2, SH3, SOCS, SPRY, START, SWIRM, TIR, TPR, TRAF, SNARE, TUBBY, TUDOR, UBA, UEV, UIM, VHL, VHS, WD40, WW, SH2, SH3, TRAF, Bromodomain, and TPR.

In some embodiments, the target protein is a heat shock protein selected from Hsp20, Hsp27, Hsp70, Hsp84, alpha B crystalline, TRAP-1, hsf1, and Hsp90.

In some embodiments, the target protein is an ion channel selected from Cav2.2, Cav3.2, IKACh, Kv1.5, TRPA1, NAv1.7, Nav1.8, Nav1.9, P2X3, or P2X4.

In some embodiments, the target protein is a coiled-coil protein selected from geminin, SPAG4, VAV1, MAD1, ROCK1, RNF31, NEDP1, HCCM, EEA1, Vimentin, ATF4, Nemo, SNAP25, Syntaxin 1a, FYCO1, and CEP250.

In some embodiments, the target protein is a kinase selected from ABL, ALK, AXL, BTK, EGFR, FMS, FAK, FGFR1, 2, 3, 4, FLT3, HER2/ErbB2, HER3/ErbB3, HER4/ErbB4, IGF1R, INSR, JAK1, JAK2, JAK3, KIT, MET, PDGFRA, PDGFRB, RET RON, ROR1, ROR2, ROS, SRC, SYK, TIE1, TIE2, TRKA, TRKB, KDR, AKT1, AKT2, AKT3, PDK1, PKC, RHO, ROCK1, RSK1, RKS2, RKS3, ATM, ATR, CDK1, CDK2, CDK3, CDK4, CDK5, CDK6, CDK7, CDK8, CDK9, CDK10, ERK1, ERK2, ERK3, ERK4, GSK3A, GSK3B, JNK1, JNK2, JNK3, AurA, ARuB, PLK1, PLK2, PLK3, PLK4, IKK, KIN1, cRaf, PKN3, c-Src, Fak, PyK2, and AMPK.

In some embodiments, the target protein is a phosphatase selected from WIP1, SHP2, SHP1, PRL-3, PTP1B, and STEP.

In some embodiments, the target protein is a ubiquitin ligase selected from BMI-1, MDM2, NEDD4-1, Beta-TRCP, SKP2, E6AP, and APC/C.

In some embodiments, the target protein is a chromatin modifier/remodeler encoded by a gene selected from BRG1, BRM, ATRX, PRDM3, ASH1L, CBP, KAT6A, KAT6B, MLL, NSD1, SETD2, EP300, KAT2A, and CREBBP.

In some embodiments, the target protein is a transcription factor encoded by a gene selected from EHF, ELF1, ELF3, ELF4, ELF5, ELK1, ELK3, ELK4, ERF, ERG, ETS1, ETV1, ETV2, ETV3, ETV4, ETV5, ETV6, FEV, FLI1, GAVPA, SPDEF, SP11, SPIC, SPIB, E2F1, E2F2, E2F3, E2F4, E2F7, E2F8, ARNTL, BHLHA15, BHLHB2, BHLBHB3, BHLHE22, BHLHE23, BHLHE41, CLOCK, FIGLA, HAS5, HES7, HEY1, HEY2, ID4, MAX, MESP1, MLX, MLXIPL, MNT, MSC, MYF6, NEUROD2, NEUROG2, NHLH1, OLIG1, OLIG2, OLIG3, SREBF2, TCF3, TCF4, TFAP4, TFE3, TFEB, TFEC, USF1, ARF4, ATF7, BATF3, CEBPB, CEBPD, CEBPG, CREB3, CREB3L1, DBP, HLF, JDP2, MAFF, MAFG, MAFK, NRL, NFE2, NFIL3, TEF, XBP1, PROX1, TEAD1, TEAD3, TEAD4, ONECUT3, ALX3, ALX4, ARX, BARHL2, BARX, BSX, CART1, CDX1, CDX2, DLX1, DLX2, DLX3, DLX4, DLX5, DLX6, DMBX1, DPRX, DRGX, DUXA, EMX1, EMX2, EN1, EN2, ESX1, EVX1, EVX2, GBX1, GBX2, GSC, GSC2, GSX1, GSX2, HESX1, HMX1, HMX2, HMX3, HNF1A, HNF1B, HOMEZ, HOXA1, HOXA10, HOXA13, HOXA2, HOXAB13, HOXB2, HOXB3, HOXB5, HOXC10, HOXC11, HOXC12, HOXC13, HOXD11, HOXD12, HOXD13, HOXD8, IRX2, IRX5, ISL2, ISX, LBX2, LHX2, LHX6, LHX9, LMX1A, LMX1B, MEIS1, MEIS2, MEIS3, MEOX1, MEOX2, MIXL1, MNX1, MSX1, MSX2, NKX2-3, NKX2-8, NKX3-1, NKX3-2, NKX6-1, NKX6-2, NOTO, ONECUT1, ONECUT2, OTX1, OTX2, PDX1, PHOX2A, PHOX2B, PITX1, PITX3, PKNOX1, PROP1, PRRX1, PRRX2, RAX, RAXL1, RHOXF1, SHOX, SHOX2, TGIF1, TGIF2, TGIF2LX, UNCX, VAX1, VAX2, VENTX, VSX1, VSX2, CUX1, CUX2, POU F1, POU2F1, POU2F2, POU2F3, POU3F1, POU3F2, POU3F3, POU3F4, POU4F1, POU4F2, POU4F3, POU5F1P1, POU6F2, RFX2, RFX3, RFX4, RFX5, TFAP2A, TFAP2B, TFAP2C, GRHL1, TFCP2, NFIA, NFIB, NFIX, GCM1, GCM2, HSF1, HSF2, HSF4, HSFY2, EBF1, IRF3, IRF4, IRF5, IRF7, IRF8, IRF9, MEF2A, MEF2B, MEF2D, SRF, NRF1, CPEB1, GMEB2, MYBL1, MYBL2, SMAD3, CENPB, PAX1, PAX2, PAX9, PAX3, PAX4, PAX5, PAX6, PAX7, BCL6B, EGR1, EGR2, EGR3, EGR4, GLIS1, GLIS2, GLI2, GLIS3, HIC2, HINFP1, KLF13, KLF14, KLF16, MTF1, PRDM1, PRDM4, SCRT1, SCRT2, SNAI2, SP1, SP3, SP4, SP8, YY1, YY2, ZBED1, ZBTB7A, ZBTB7B, ZBTB7C, ZIC1, ZIC3, ZIC4, ZNF143, ZNF232, ZNF238, ZNF282, ZNF306, ZNF410, ZNF435, ZBTB49, ZNF524, ZNF713, ZNF740, ZNF75A, ZNF784, ZSCAN4, CTCF, LEF1, SOX10, SOX14, SOX15, SOX18, SOX2, SOX21, SOX4, SOX7, SOX8, SOX9, SRY, TCF7L1, FOXO3, FOXB1, FOXC1, FOXC2, FOXD2, FOXD3, FOXG1, FOXI1, FOXJ2, FOXJ3, FOXK1, FOXL1, FOXO1, FOXO4, FOXO6, FOXP3, EOMES, MGA, NFAT5, NFATC1, NFKB1, NFKB2, TP63, RUNX2, RUNX3, T, TBR1, TBX1, TBX15, TBX19, TBX2, TBX20, TBX21, TBX4, TBX5, AR, ESR1, ESRRA, ESRRB, ESRRG, HNF4A, NR2C₂, NR2E1, NR2F1, NR2F6, NR3C₁, NR3C₂, NR4A2, RARA, RARB, RARG, RORA, RXRA, RXRB, RXRG, THRA, THRB, VDR, GATA3, GATA4, GATA5, C-myc, Max, Stat3, androgen receptor, C-Jun, C-Fox, N-Myc, L-Myc, MITF, Hif-1alpha, Hif-2alpha, Bcl6, E2F1, NF-kappaB, Stat5, and ER(coact).

In some embodiments, the target protein is selected from the group consisting of TrkA, P2Y14, mPEGS, ASK1, ALK, Bcl-2, BCL-XL, mSIN1, RORγt, IL17RA, eIF4E, TLR7R, PCSK9, IgE R, CD40, CD40L, Shn-3, TNFR1, TNFR2, IL31RA, OSMR, IL12beta1,2, Tau, FASN, KCTD 6, KCTD 9, Raptor, Rictor, RALGAPA, RALGAPB, Annexin family members, BCOR, NCOR, beta catenin, AAC 11, PLD1, PLD2, Frizzled7, RaLP, MLL-1, Myb, Ezh2, RhoGD12, EGFR, CTLA4R, GCGC (coact), Adiponectin R2, GPR 81, IMPDH2, IL-4R, IL-13R, IL-1R, IL2-R, IL-6R, IL-22R, TNF-R, TLR4, Nrlp3, and OTR.

In another aspect, the present disclosure provides a presenter protein/compound complex comprising a presenter protein and a compound described herein.

In some embodiments of the tri-complexes or the presenter protein/compound complexes described herein, the presenter protein is a prolyl isomerase. In some embodiments, the presenter protein is a member of the FKBP family, a member of the cyclophilin family, or PIN1. In some embodiments, the presenter protein is a member of the FKBP family selected from FKBP12, FKBP12.6, FKBP13, FKBP19, FKBP22, FKBP23, FKBP25, FKBP36, FKBP38, FKBP51, FKBP52, FKBP60, FKBP65, and FKBP133. In some embodiments, the member of the FKBP family is FKBP12, FKBP12.6, FKBP25, or FKBP52. In some embodiments, the presenter protein is a member of the cyclophilin family selected from PP1A, CYPB, CYPC, CYP40, CYPE, CYPD, NKTR, SRCyp, CYPH, CWC27, CYPL1, CYP60, CYPJ, PPIL4, PPIL6, RANBP2, PPWD1, PPIAL4A, PPIAL4B, PPIAL4C, PPIAL4D, and PPIAL4G. In some embodiments, the member of the cyclophilin family is PPIAL4A, PPIAL4B, PPIAL4C, PPIAL4D, or PPIAL4G.

In another aspect, the disclosure provides a method of modulating a target protein by contacting the target protein with a compound or presenter protein/compound complex described herein.

In a further aspect, the disclosure provides a method of inhibiting a target protein by contacting the target protein with a compound or presenter protein/compound complex described herein.

In still another aspect, the disclosure provides a method of activating a target protein by contacting the target protein with a compound or presenter protein/compound complex described herein.

In another aspect, the disclosure provides a method of forming a tri-complex described herein by contacting a target protein with a presenter protein/compound complex described herein.

In some embodiments of the foregoing methods, upon contacting the target protein, the target protein forms a covalent bond to the compound or the presenter protein/compound complex. In some embodiments, upon contacting the target protein, an aspartic acid, glutamic acid, cysteine, glutamine, asparagine, lysine, or histidine residue of the target protein forms a covalent bond to the compound or the complex. In some embodiments, upon contacting the target protein, an aspartic acid, glutamic acid, cysteine, glutamine, or asparagine residue of the target protein forms a covalent bond to the compound or the complex.

In another aspect, the disclosure provides a method of crosslinking a compound described herein to a second moiety by contacting the second moiety with the compound under conditions sufficient to form a covalent bond between the compound and the second moiety. Such conditions include sufficient orientation and residency time for the compound and the second moiety to form a covalent bond.

Methods of determining if a covalent bond has formed are known in the art, such as using FRET, mass spectrometry, or a gel-shift assay. In some embodiments, the second moiety is a target protein.

In a further aspect, the disclosure provides a method of forming a presenter protein/compound complex described herein by contacting a presenter protein with a compound described herein under conditions sufficient to permit the formation of a complex. In some embodiments, the complex is formed by way of noncovalent interactions. Methods of measuring such interactions are known in the art, such as using FRET.

In another aspect, the disclosure provides a method of forming a tri-complex described herein, including the following steps:
a) contacting a presenter protein with the compound described herein under conditions sufficient to permit the formation of presenter protein/compound complex; and
b) contacting the presenter protein/compound complex with a target protein under conditions that permit the formation of a tri-complex.

In some embodiments, the presenter protein/compound complex binds to the target protein with at least 5-fold greater affinity than the presenter protein or the compound alone. In some embodiments, the presenter protein or the compound do not substantially bind to the target protein in the absence of forming the presenter protein/compound complex.

In yet another aspect, the disclosure provides a method of treating a disease or disorder in a subject in need thereof by administering to the subject a therapeutically effective amount of a compound described herein. In some embodiments, the subject has previously been treated with a prior therapy, such as a cancer therapy. In some embodiments, the subject has developed resistance to treatment with a prior therapy, such as a cancer therapy.

It is specifically contemplated that any limitation discussed with respect to one embodiment of the disclosure may apply to any other embodiment of the disclosure. Furthermore, any compound or composition of the disclosure may be used in any method of the disclosure, and any method of the disclosure may be used to produce or to utilize any compound or composition of the disclosure.

### Definitions and Chemical Terms

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used herein, the term “adjacent” in the context of describing adjacent atoms refers to bivalent atoms that are directly connected by a covalent bond.

A “compound of the present invention,” “a compound disclosed herein,” or “a compound described herein” and similar terms as used herein, whether explicitly noted or not, refers to compounds of Formula I or Formula II and subformulae thereof, and compounds of Table 3, as well as salts (e.g., pharmaceutically acceptable salts), solvates, hydrates, stereoisomers (including atropisomers), and tautomers thereof.

The term “wild-type” refers to an entity having a structure or activity as found in nature in a “normal” (as contrasted with mutant, diseased, altered, etc.) state or context. Those of ordinary skill in the art will appreciate that wild-type genes and polypeptides often exist in multiple different forms (e.g., alleles).

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation state having the same empirical formula and total charge as a reference form. Examples of moieties with prototropic tautomeric forms are ketone—enol pairs, amide—imidic acid pairs, lactam—lactim pairs, amide—imidic acid pairs, enamine—imine pairs, and annular forms where a proton can occupy two or more positions of a heterocyclic system, such as, 1H- and 3H-imidazole, 1H—, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent.

As is known in the art, many chemical entities can adopt a variety of different solid forms such as, for example, amorphous forms or crystalline forms (e.g., polymorphs, hydrates, solvate). In some embodiments, compounds of the present invention may be utilized in any such form, including in any solid form. In some embodiments, compounds described or depicted herein may be provided or utilized in hydrate or solvate form.

Non-limiting examples of moieties that may contain one or more deuterium substitutions in compounds of the present invention, where any position “R” may be deuterium (D), include

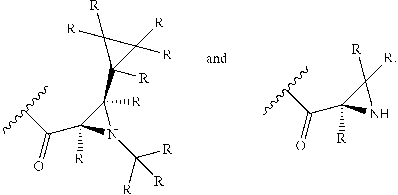

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substituents are disclosed in groups or in ranges, unless otherwise indicated, the present disclosure is intended to cover individual compounds and groups of compounds (e.g., genera and subgenera) containing each and every individual subcombination of members at each position.

The term “optionally substituted X” (e.g., “optionally substituted alkyl”) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₉ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be, independently, deuterium; halogen; —(CH₂)₀₋₄R^{º}; —(CH₂)₀₋₄OR^{º}; —O(CH₂)₀₋₄R^{º}; —O—(CH₂)₀₋₄C(O)OR^{º}; —(CH₂)₀₋₄CH(OR^{º})₂; —(CH₂)₀₋₄SR^{º}; —(CH₂)₀₋₄Ph, which may be substituted with R^{º}; —(CH₂)₀₋₄O(CH₂)₀₋₁Ph which may be substituted with R^{º}; —CH═CHPh, which may be substituted with R^{º}; —(CH₂)₀₋₄O(CH₂)₀₋₁-pyridyl which may be substituted with R^{º}; 4-11 membered saturated or unsaturated heterocycloalkyl (e.g., 4-8 membered saturated or unsaturated heterocycloalkyl (e.g., pyridyl)) which may be further optionally substituted (e.g., with a methyl); 3-8 membered saturated or unsaturated cycloalkyl (e.g., cyclopropyl, cyclobutyl, or cyclopentyl); —NO₂; —CN; —N₃; —(CH₂)₀₋₄N(R^{º})₂; —(CH₂)₀₋₄N(R^{º})C(O)R^{º}; —N(R^{º})C(S)R^{º}; —(CH₂)₀₋₄N(R^{º})C(O)NR^{º}₂; —N(R^{º})C(S)NR^{º}₂; —(CH₂)₀₋₄N(R^{º})C(O)OR^{º}; —N(R^{º})N(R^{º})C(O)R^{º}; —N(R^{º})N(R^{º})C(O)NR^{º}₂; —N(R^{º})N(R^{º})C(O)OR^{º}; —(CH₂)₀₋₄C(O)R^{º}; —C(S)R^{º}; —(CH₂)₀₋₄C(O)OR^{º}; —(CH₂)₀₋₄—C(O)—N(R^{º})₂; —(CH₂)₀₋₄—C(O)—N(R^{º})—S(O)₂—R^{º}; —C(NCN)NR^{º}₂; —(CH₂)₀₋₄C(O)SR^{º}; —(CH₂)₀₋₄C(O)OSiR^{º}₃; —(CH₂)₀₋₄OC(O)R^{º}; —OC(O)(CH₂)₀₋₄SR^{º}; —SC(S)SR^{º}; —(CH₂)₀₋₄SC(O)R^{º}; —(CH₂)₀₋₄C(O)NR^{º}₂; —C(S)NR^{º}₂; —C(S)SR^{º}; —(CH₂)₀₋₄OC(O)NR^{º}₂; —C(O)N(OR^{º})R^{º}; —C(O)C(O)R^{º}; —C(O)CH₂C(O)R^{º}; —C(NOR^{º})R^{º}; —(CH₂)₀₋₄SSR^{º}; —(CH₂)₀₋₄S(O)₂R^{º}; —(CH₂)₀₋₄S(O)₂₀R^{º}; —(CH₂)₀₋₄OS(O)₂R^{º}; —S(O)₂NR^{º}₂; —(CH₂)₀₋₄S(O)R^{º}; —N(R^{º})S(O)₂NR^{º}₂; —N(R^{º})S(O)₂R^{º}; —N(OR^{º})R^{º}; —C(NOR^{º})NR^{º}₂; —C(NH)NR^{º}₂; —P(O)₂R^{º}; —P(O)R^{º}₂; —P(O)(OR^{º})₂; —OP(O)R^{º}₂; —OP(O)(OR^{º})₂; —OP(O)(OR^{º})R^{º}, —SiR^{º}₃; —(C₁₋₄ straight or branched alkylene)O—N(R^{º})₂; or —(C₁₋₄ straight or branched alkylene)C(O)O—N(R^{º})₂, wherein each R^{º} may be substituted as defined below and is independently hydrogen, —C₁₋₆ aliphatic, —CH₂Ph, —O(CH₂)₀₋₁Ph, —CH₂-(5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R^{º}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below. In some embodiments, a substituent of a suitable carbon is —N₃, such as for the purpose of click chemistry reactions, as described herein.

Suitable monovalent substituents on R^{º} (or the ring formed by taking two independent occurrences of R^{º} together with their intervening atoms), may be, independently, halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂ C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NH R^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{º} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on an aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{†} include ═O and ═S.

The term “acetyl,” as used herein, refers to the group —C(O)CH₃.

The term “acyl,” as used herein, refers to the group —C(O)—R, where R is alkyl, alkenyl, alkynyl, heteroalkyl, heteroalkenyl, heteroalkynyl, cycloalkyl, cycloalkenyl, cycloalkynyl, heterocycloalkyl, heterocycloalkenyl, heterocycloalkynyl, or heteroaryl, where each group is as defined herein. An optionally substituted acyl is an acyl that is optionally substituted as defined herein for each group.

The term “alkoxy,” as used herein, refers to a —O—C₁-C₂₀ alkyl group, wherein the alkoxy group is attached to the remainder of the compound through an oxygen atom.

The term “alkyl,” as used herein, refers to a saturated, straight or branched monovalent hydrocarbon group containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by, but not limited to, methyl, ethyl, n- and iso-propyl, n-, sec-, iso- and tert-butyl, and neopentyl.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like. The term “Cₓ-C_{y} alkylene” represents alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁-C₆, C₁-C₁₀, C₂-C₂₀, C₂-C₆, C₂-C₁₀, or C₂-C₂₀ alkylene). In some embodiments, the alkylene can be further substituted with 1, 2, 3, or 4 substituent groups as defined herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, and 2-butenyl. Alkenyls include both cis and trans isomers. The term “alkenylene,” as used herein, represents a divalent straight or branched chain groups of, unless otherwise specified, from 2 to 20 carbons (e.g., from 2 to 6 or from 2 to 10 carbons) containing one or more carbon-carbon double bonds.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, and 1-propynyl.

The term “alkynyl sulfone,” as used herein, represents a group comprising the structure

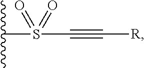

wherein R is any chemically feasible substituent described herein.

The term “amino,” as used herein, represents —N(R^{†})₂, e.g., —NH₂ and —N(CH₃)₂.

The term “aminoalkyl,” as used herein, represents an alkyl moiety substituted on one or more carbon atoms with one or more amino moieties.

The term “amino acid,” as described herein, refers to a molecule having a side chain, an amino group, and an acid group (e.g., —CO₂H or —SO₃H), wherein the amino acid is attached to the parent molecular group by the side chain, amino group, or acid group (e.g., the side chain). As used herein, the term “amino acid” in its broadest sense, refers to any compound or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. Exemplary amino acids include alanine, arginine, asparagine, aspartic acid, cysteine, glutamic acid, glutamine, glycine, histidine, optionally substituted hydroxynorvaline, isoleucine, leucine, lysine, methionine, norvaline, ornithine, phenylalanine, proline, pyrrolysine, selenocysteine, serine, taurine, threonine, tryptophan, tyrosine, and valine.

The term “aryl,” as used herein, represents a monovalent monocyclic, bicyclic, or multicyclic ring system formed by carbon atoms, wherein the ring attached to the pendant group is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified.

The term “C₀,” as used herein, represents a bond. For example, part of the term —N(C(O)—(C₀-C₅ alkylene-H)— includes —N(C(O)—(C₀ alkylene-H)—, which is also represented by —N(C(O)—H)—.

The terms “carbocyclic” and “carbocyclyl,” as used herein, refer to a monovalent, optionally substituted C₃-C₁₂ monocyclic, bicyclic, or tricyclic ring structure, which may be bridged, fused or spirocyclic, in which all the rings are formed by carbon atoms and at least one ring is non-aromatic. Carbocyclic structures include cycloalkyl, cycloalkenyl, and cycloalkynyl groups. Examples of carbocyclyl groups are cyclohexyl, cyclohexenyl, cyclooctynyl, 1,2-dihydronaphthyl, 1,2,3,4-tetrahydronaphthyl, fluorenyl, indenyl, indanyl, decalinyl, and the like. A carbocyclic ring can be attached to its pendant group at any ring atom that results in a stable structure and any of the ring atoms can be optionally substituted unless otherwise specified.

The term “carbonyl,” as used herein, represents a C(O) group, which can also be represented as C═O.

The term “carboxyl,” as used herein, means —CO₂H, (C═O)(OH), COOH, or C(O)OH or the unprotonated counterparts.

The term “cyano,” as used herein, represents a —CN group.

The term “cycloalkyl,” as used herein, represents a saturated cyclic hydrocarbon group, which may be bridged, fused or spirocyclic having from three to eight ring carbons, unless otherwise specified, and is exemplified by cyclopropyl, cyclobutyl, cyclopentyl, cyclohexyl, cycloheptyl, and cycloheptyl.

The term “cycloalkenyl,” as used herein, represents a non-aromatic, saturated cyclic hydrocarbon group, which may be bridged, fused or spirocyclic having from three to eight ring carbons, unless otherwise specified, and containing one or more carbon-carbon double bonds.

The term “diastereomer,” as used herein, means stereoisomers that are not mirror images of one another and are non-superimposable on one another.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “haloacetyl,” as used herein, refers to an acetyl group wherein at least one of the hydrogens has been replaced by a halogen.

The term “haloalkyl,” as used herein, represents an alkyl moiety substituted on one or more carbon atoms with one or more of the same of different halogen moieties.

The term “halogen,” as used herein, represents a halogen selected from bromine, chlorine, iodine, or fluorine.

The term “heteroalkyl,” as used herein, refers to an “alkyl” group, as defined herein, in which at least one carbon atom has been replaced with a heteroatom (e.g., an O, N, or S atom). The heteroatom may appear in the middle or at the end of the radical. The term “heteroalkylene,” as used herein, represents a divalent alkylene straight or branched chain groups of, unless otherwise specified, from 2 to 20 carbons (e.g., from 2 to 6 or from 2 to 10 carbons) in which at least one carbon atom has been replaced with a heteroatom (e.g., an O, N, or S atom). The heteroatom may appear in the middle or at the end of the radical.

The term “heteroalkenyl,” as used herein, refers to an “alkenyl” group, as defined herein, in which at least one carbon atom has been replaced with a heteroatom (e.g., an O, N, or S atom). The heteroatom may appear in the middle or at the end of the radical.

The term “heteroalkynyl,” as used herein, refers to an “alkynyl” group, as defined herein, in which at least one carbon atom has been replaced with a heteroatom (e.g., an O, N, or S atom). The heteroatom may appear in the middle or at the end of the radical.

The term “heteroaryl,” as used herein, represents a monovalent, monocyclic or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups include, but are not limited to, pyridyl, pyrazolyl, benzooxazolyl, benzoimidazolyl, benzothiazolyl, imidazolyl, thiazolyl, quinolinyl, tetrahydroquinolinyl, and 4-azaindolyl. A heteroaryl ring can be attached to its pendant group at any ring atom that results in a stable structure and any of the ring atoms can be optionally substituted unless otherwise specified. In some embodiments, the heteroaryl is substituted with 1, 2, 3, or 4 substituents groups.

The term “heterocycloalkyl,” as used herein, represents a monocyclic, bicyclic or polycyclic ring system, which may be bridged, fused or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocycloalkyl groups are of 1 to 12 (e.g., 1 to 11, 1 to 10, 1 to 9, 2 to 12, 2 to 11, 2 to 10, or 2 to 9) carbons. The term “heterocycloalkyl” also represents a heterocyclic compound having a bridged multicyclic structure in which one or more carbons or heteroatoms bridges two non-adjacent members of a monocyclic ring, e.g., a quinuclidinyl group. The term “heterocycloalkyl” includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. Examples of heterocycloalkyl groups are pyrrolidinyl, piperidinyl, 1,2,3,4-tetrahydroquinolinyl, decahydroquinolinyl, dihydropyrrolopyridine, and decahydronapthyridinyl. A heterocycloalkyl ring can be attached to its pendant group at any ring atom that results in a stable structure and any of the ring atoms can be optionally substituted unless otherwise specified.

The term “heterocycloalkenyl,” as used herein, represents a non-aromatic, saturated cyclic heterocyclic group, which may be bridged, fused or spirocyclic having from five to ten ring atoms, unless otherwise specified, containing one or more carbon-carbon double bonds, and containing one, two, three, or four heteroatoms independently selected from the group consisting of nitrogen, oxygen, and sulfur.

The term “heterocycloalkynyl,” as used herein, represents a non-aromatic, saturated cyclic heterocyclic group, which may be bridged, fused or spirocyclic having from eight to ten ring atoms, unless otherwise specified, containing a carbon-carbon triple bond, and containing one, two, three, or four heteroatoms independently selected from the group consisting of nitrogen, oxygen, and sulfur.

The term “hydroxy,” as used herein, represents a —OH group.

The term “hydroxyalkyl,” as used herein, represents an alkyl moiety substituted on one or more carbon atoms with one or more —OH moieties.

The term “isomer,” as used herein, means any tautomer, stereoisomer, atropisomer, enantiomer, or diastereomer of any compound of the invention. It is recognized that the compounds of the invention can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used herein, the term “linker” refers to a divalent organic moiety connecting a first moiety (e.g., a macrocyclic moiety) to a second moiety (e.g., a cross-linking group).

A “macrocyclic moiety” or “macrocycle” refers to a compound or a portion of a compound that has a ring of at least 10 atoms (e.g., at least 12, at least 14, at least 16, from 10 to 40, from 12 to 40, or from 12 to 30). In some embodiments, the macrocycle has at least 12 atoms. In some embodiments, the macrocycle has at least 14 atoms.

In some embodiments, the linker comprises 20 or fewer linear atoms. In some embodiments, the linker comprises 15 or fewer linear atoms. In some embodiments, the linker comprises 10 or fewer linear atoms. In some embodiments, the linker has a molecular weight of under 500 g/mol. In some embodiments, the linker has a molecular weight of under 400 g/mol. In some embodiments, the linker has a molecular weight of under 300 g/mol. In some embodiments, the linker has a molecular weight of under 200 g/mol. In some embodiments, the linker has a molecular weight of under 100 g/mol. In some embodiments, the linker has a molecular weight of under 50 g/mol.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers or conformers of the basic molecular structure, including atropisomers. Some compounds of the present invention may exist in different tautomeric forms, all of the latter being included within the scope of the present invention.

The term “sulfonyl,” as used herein, represents an —S(O)₂— group.

The term “thiocarbonyl,” as used herein, refers to a —C(S)— group.

Those of ordinary skill in the art, reading the present disclosure, will appreciate that certain compounds described herein may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form.

Chemical substituents may be “capable of stabilizing a positive charge or partial positive charge” or “carbocation stabilizing” in ways known to those skilled in the art of organic chemistry. For example, a substituent may be capable of stabilizing a positive charge through a resonance effect (delocalization of electron density throughout adjacent orbitals), hyperconjugation (e.g., interaction of electrons in a sigma orbital with an adjacent non-bonding p orbital), inductive effect (changes in electron density due to electron withdrawing or electron donating groups in the molecule).

As used herein, the term “complex” refers to a group of two or more compounds and/or proteins which are bound together through a binding interaction (e.g., a covalent bond or a non-covalent interaction, such as a hydrophobic effect interaction, an electrostatic interaction, a van der Waals interaction, or π-effect interaction). Examples of complexes are a “presenter protein/compound complex” which include a compound of the invention bound to a presenter protein and a “compound/target protein complex” which include a compound of the invention bound to a target protein.

The term “presenter protein” refers to a protein that binds to a small molecule to form a complex that binds to and modulates the activity of a target protein (e.g., a eukaryotic target protein such as a mammalian target protein or a fungal target protein or a prokaryotic target protein such as a bacterial target protein). In some embodiments, the presenter protein is a relatively abundant protein (e.g., the presenter protein is sufficiently abundant that participation in a tri-complex does not substantially impact the biological role of the presenter protein in a cell and/or viability or other attributes of the cell). In certain embodiments, the presenter protein is a protein that has chaperone activity within a cell. In some embodiments, the presenter protein is a protein that has multiple natural interaction partners within a cell. In certain embodiments, the presenter protein is one which is known to bind a small molecule to form a binary complex that is known to or suspected of binding to and modulating the biological activity of a target protein.

The term “presenter protein binding moiety” refers to a group of ring atoms and the moieties attached thereto (e.g., atoms within 20 atoms of a ring atom such as, atoms within 15 atoms of a ring atom, atoms within 10 atoms of a ring atom, atoms within 5 atoms of a ring atom) that participate in binding to a presenter protein such that the compound specifically binds to said presenter protein, for example, with a K_{D} of less than 10 μM (e.g., less than 5 μM, less than 1 μM, less than 500 nM, less than 200 nM, less than 100 nM, less than 75 nM, less than 50 nM, less than 25 nM, less than 10 nM) or inhibits the peptidyl-prolyl isomerase activity of the presenter protein, for example, with an IC₅₀ of less than 1 μM (e.g., less than 0.5 μM, less than 0.1 μM, less than 0.05 μM, less than 0.01 μM). It will be understood that the presenter protein binding moiety does not necessarily encompass the entirety of atoms in the compound that interact with the presenter protein. It will also be understood that one or more atoms of the presenter protein binding moiety may be within the target protein interaction moiety (e.g., eukaryotic target protein interacting moiety such as mammalian target protein interacting moiety or fungal target protein interacting moiety or prokaryotic target protein interacting moiety such as a bacterial target protein interacting moiety). In some embodiments, the presenter protein binding moiety has a molecular weight of under 1000 g/mol. In some embodiments, the presenter protein binding moiety has a molecular weight of under 750 g/mol. In some embodiments, the presenter protein binding moiety has a molecular weight of under 500 g/mol. In some embodiments, the presenter protein binding moiety has a molecular weight of under 400 g/mol. In some embodiments, the presenter protein binding moiety has a molecular weight of under 300 g/mol. In some embodiments, the presenter protein binding moiety has a molecular weight of under 200 g/mol. In some embodiments, the presenter protein binding moiety has a molecular weight of under 100 g/mol. In some embodiments, the presenter protein binding moiety has a molecular weight of under 50 g/mol.

It will be understood that the term “binding” as used herein, typically refers to association (e.g., non-covalent or covalent) between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

The affinity of a molecule X for its partner Y can generally be represented by the dissociation constant (K_{D}). Affinity can be measured by common methods known in the art, including those described herein. Specific illustrative and exemplary embodiments for measuring binding affinity are described below. The term “K_{D},” as used herein, is intended to refer to the dissociation equilibrium constant of a particular compound-protein or complex-protein interaction. Typically, the compounds of the invention bind to presenter proteins with a dissociation equilibrium constant (K_{D}) of less than about 10⁻⁶ M, such as less than approximately 10⁻⁷ M, 10⁻⁸ M, 10⁻⁹ M, or 10⁻¹⁰ M or even lower, e.g., when determined by surface plasmon resonance (SPR) technology using the presenter protein as the analyte and the compound as the ligand. The presenter protein/compound complexes of the invention bind to target proteins (e.g., a eukaryotic target protein such as a mammalian target protein or a fungal target protein or a prokaryotic target protein such as a bacterial target protein) with a dissociation equilibrium constant (K_{D}) of less than about 10⁻⁶ M, such as less than approximately 10⁻⁷ M, 10⁻⁸ M, 10⁻⁹ M, or 10⁻¹⁰ M or even lower, e.g., when determined by surface plasmon resonance (SPR) technology using the target protein as the analyte and the complex as the ligand.

The term “target protein interacting moiety” as used herein, refers to a group of ring atoms and the moieties attached thereto (e.g., atoms within 20 atoms of a ring atom such as, atoms within 15 atoms of a ring atom, atoms within 10 atoms of a ring atom, atoms within 5 atoms of a ring atom) that, when the compound is in a complex with a presenter protein, specifically bind to a target protein (e.g., a eukaryotic target protein such as a mammalian target protein or a fungal target protein or a prokaryotic target protein such as a bacterial target protein).

The term “modulator” is used to refer to an entity whose presence or level in a system in which an activity of interest is observed correlates with a change in level and/or nature of that activity as compared with that observed under otherwise comparable conditions when the modulator is absent. In some embodiments, a modulator is an activator, in that activity is increased in its presence as compared with that observed under otherwise comparable conditions when the modulator is absent. In some embodiments, a modulator is an antagonist or inhibitor, in that activity is reduced in its presence as compared with otherwise comparable conditions when the modulator is absent. In some embodiments, a modulator interacts directly with a target entity whose activity is of interest. In some embodiments, a modulator interacts indirectly (i.e., directly with an intermediate compound that interacts with the target entity) with a target entity whose activity is of interest. In some embodiments, a modulator affects the level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level. In some embodiments, a modulator is an allosteric modulator such as an allosteric agonist.

The term “presenter protein” refers to a protein that binds to a small molecule to form a complex that binds to and modulates the activity of a target protein (e.g., a eukaryotic target protein such as a mammalian target protein or a fungal target protein or a prokaryotic target protein such as a bacterial target protein). In some embodiments, the presenter protein is a relatively abundant protein (e.g., the presenter protein is sufficiently abundant that participation in a tri-complex does not substantially impact the biological role of the presenter protein in a cell and/or viability or other attributes of the cell). In certain embodiments, the presenter protein is a protein that has chaperone activity within a cell. In some embodiments, the presenter protein is a protein that has multiple natural interaction partners within a cell. In certain embodiments, the presenter protein is one which is known to bind a small molecule to form a binary complex that is known to or suspected of binding to and modulating the biological activity of a target protein.

The term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

The term “does not substantially bind” to a particular protein as used herein can be exhibited, for example, by a molecule or portion of a molecule having a K_{D} for the target of 10⁻⁴ M or greater, alternatively 10⁻⁵ M or greater, alternatively 10⁻⁶ M or greater, alternatively 10⁻⁷ M or greater, alternatively 10⁻⁸ M or greater, alternatively 10⁻⁹ M or greater, alternatively 10⁻¹⁰ M or greater, alternatively 10⁻¹¹ M or greater, alternatively 10⁻¹² M or greater, or a K_{D} in the range of 10⁻⁴ M to 10⁻¹² M or 10⁻⁶ M to 10⁻¹⁰ M or 10⁻⁷ M to 10⁻⁹ M.

The term “target protein” refers to a protein that binds with a small molecule, or a presenter protein/compound complex as described herein. In some embodiments, the target protein does not substantially bind with either the small molecule or the presenter protein alone. In some embodiments, the small molecule/presenter protein/compound complex does not substantially bind to mTOR or calcineurin. In some embodiments, the target protein participates in a biological pathway associated with a disease, disorder or condition. In some embodiments, a target protein is a naturally-occurring protein; in some such embodiments, a target protein is naturally found in certain mammalian cells (e.g., a mammalian target protein), fungal cells (e.g., a fungal target protein), bacterial cells (e.g., a bacterial target protein) or plant cells (e.g., a plant target protein). In some embodiments, a target protein is characterized by natural interaction with one or more natural presenter protein/natural small molecule complexes. In some embodiments, a target protein is characterized by natural interactions with a plurality of different natural presenter protein/natural small molecule complexes; in some such embodiments some or all of the complexes utilize the same presenter protein (and different small molecules). In some embodiments, a target protein does not substantially bind to a complex of cyclosporin, rapamycin, or FK506 and a presenter protein (e.g., FKBP). Target proteins can be naturally occurring, e.g., wild type. Alternatively, the target protein can vary from the wild type protein but still retain biological function, e.g., as an allelic variant, a splice mutant or a biologically active fragment. Exemplary mammalian target proteins are GTPases, GTPase activating protein, Guanine nucleotide-exchange factor, heat shock proteins, ion channels, coiled-coil proteins, kinases, phosphatases, ubiquitin ligases, transcription factors, chromatin modifier/remodelers, proteins with classical protein-protein interaction domains and motifs, or any other proteins that participate in a biological pathway associated with a disease, disorder or condition.

The term “target protein interacting moiety” refers to a group of atoms in a compound that participate in binding to a target protein (e.g., a eukaryotic target protein such as a mammalian target protein or a fungal target protein or a prokaryotic target protein such as a bacterial target protein). In some embodiments, the compound binds to the target protein when the compound is in a complex with a presenter protein. It will be understood that the target protein interacting moiety does not necessarily encompass the entirety of atoms in the compound that interact with the target protein. It will also be understood that one or more atoms of the presenter protein binding moiety may also be present in the target protein interacting moiety. In some embodiments, the target protein interacting moiety is a cross-linking group.

The term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a small molecule is not a protein or polypeptide (e.g., is not an oligopeptide or peptide). In some embodiments, a small molecule is not a polynucleotide (e.g., is not an oligonucleotide). In some embodiments, a small molecule is not a polysaccharide. In some embodiments, a small molecule does not comprise a polysaccharide (e.g., is not a glycoprotein, proteoglycan, glycolipid, etc.). In some embodiments, a small molecule is not a lipid. In some embodiments, a small molecule is a modulating compound. In some embodiments, a small molecule is biologically active. In some embodiments, a small molecule is detectable (e.g., comprises at least one detectable moiety). In some embodiments, a small molecule is a therapeutic.

### DETAILED DESCRIPTION

### Compounds

The disclosure provides compounds capable of forming complexes and/or crosslinking to a target (e.g., a target protein). Also disclosed are synthetic intermediates used in the preparation of such compounds and complexes formed from reaction with the compounds.

### Synthetic Intermediates

Compounds of the present disclosure may contain an aziridine moiety. The compounds of the invention may contain an aziridine and a group capable of reaction with a second moiety allowing for the aziridine to be incorporated into another moiety. The compound may be, for example, capable of reaction with a nucleophile (e.g., an ester). Accordingly, the compounds may have the structure of Formula I:

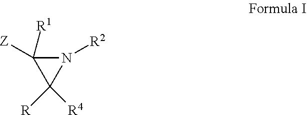

or a pharmaceutically acceptable salt thereof; wherein
Z is

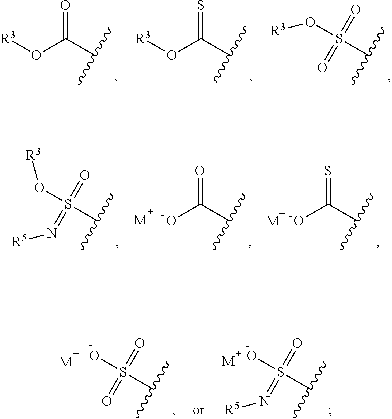

M⁺ is a cation;
R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl or —Si(R^{1a})₃; or
R and R¹ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R and R² combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and R¹ and R⁴ are each, independently, hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a}); or
R and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R² combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and each of R and R⁴ is, independently, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R² and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and each of R¹ and R is, independently, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R³ is hydrogen, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₃-C₁₀ cycloalkyl, optionally substituted C₃-C₁₀ cycloalkenyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl;
each R¹—, R²³, R^{2c}, R^{2d}, and R^{2e} is, independently, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl;
R^{2b} is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁵ is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl,
and wherein each hydrogen is independently, optionally, isotopically enriched for deuterium.

In some embodiments, at least one of R and R⁴ is not hydrogen.

In some embodiments, the compound, or pharmaceutically acceptable salt thereof, has the structure of Formula Ia:

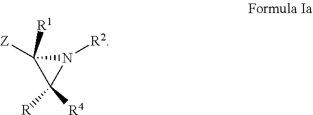

In some embodiments, the compound, or pharmaceutically acceptable salt thereof, has the structure of Formula Ib:

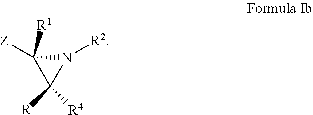

In some embodiments, the compound, or pharmaceutically acceptable salt thereof, has the structure of Formula Ic:

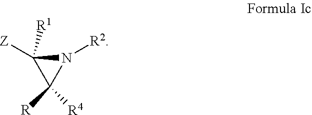

In some embodiments, the compound, or pharmaceutically acceptable salt thereof, has the structure of Formula Id:

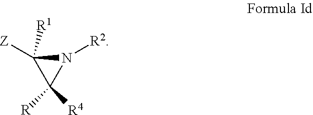

In some embodiments, R³ is optionally substituted C₁-C₆ alkyl, optionally substituted 3- to 10-membered heterocycloalkyl, optionally substituted C₆-C₁₀ aryl, or optionally substituted 5- to 10-membered heteroaryl. In some embodiments, R³ is optionally substituted C₁-C₆ alkyl. In some embodiments, R³ is methyl, ethyl, or benzyl.

In some embodiments, M⁺ is Li⁺.

In some embodiments, R³ is:

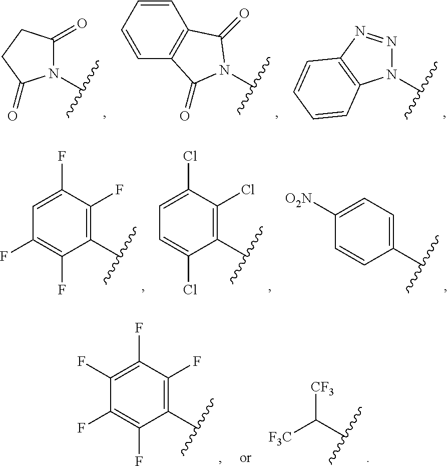

In some embodiments, Z is

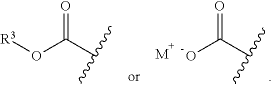

In some embodiments, the compound is a compound of Table 1, or a pharmaceutically acceptable salt, or alternative pharmaceutically acceptable salt, thereof, or a stereoisomer thereof:

TABLE 1

| Ex # | Structure |
|---|---|
| 1 | 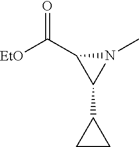 |
| 2 | 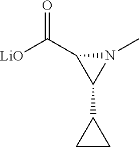 |
| 3 | 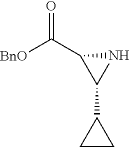 [SMILES: O=C(OCc1ccccc1)[C@@H]1N[C@@H]1C1CC1] |
| 4 | 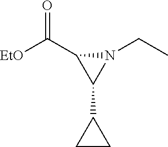 |
| 5 | 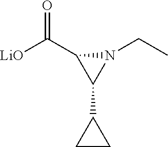 |
| 6 | 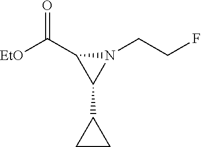 |
| 7 | 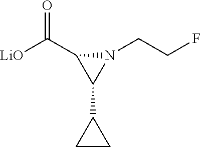 |
| 8 | 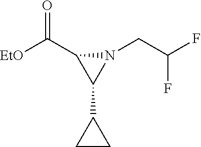 |
| 9 | 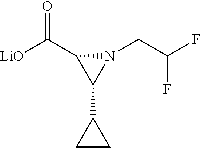 |
| 10 | 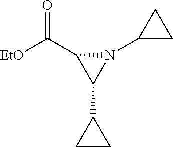 |
| 11 | 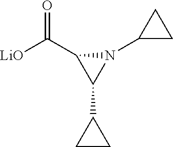 |
| 12 | 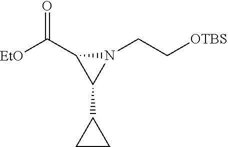 |
| 13 | 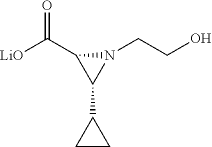 |
| 14 | 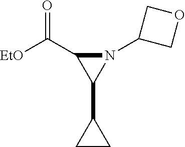 |
| 15 | 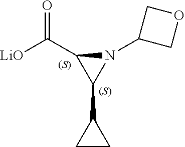 |
| 16 | 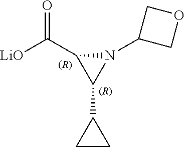 |
| 17 | 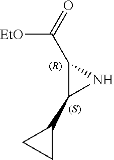 |
| 18 | 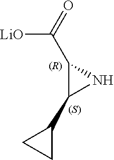 |
| 19 | 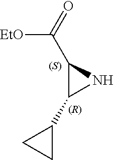 |
| 20 | 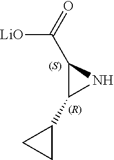 |
| 21 | 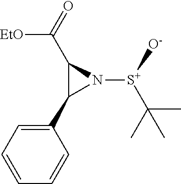 |
| 22 | 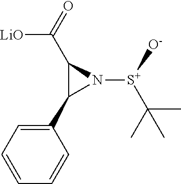 |
| 23 | 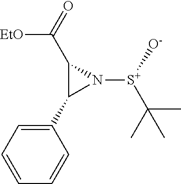 |
| 24 | 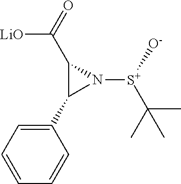 |
| 25 | 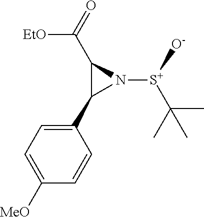 |
| 26 | 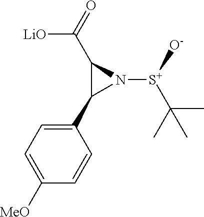 |
| 27 | 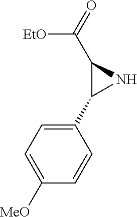 [SMILES: CCOC(=O)[C@H]1N[C@@H]1c1ccc(OC)cc1] |
| 28 | 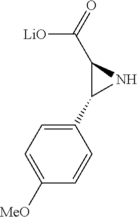 |
| 29 | 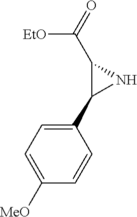 [SMILES: CCOC(=O)[C@@H]1N[C@H]1c1ccc(OC)cc1] |
| 30 | 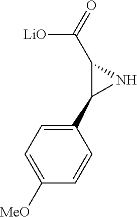 |

### Aziridine-Containing Compounds

The disclosure also features compounds containing an aziridine moiety bound to a monovalent organic moiety. Persons of skill in the art are familiar with organic moieties. The monovalent organic moiety may be or may comprise, for example, a small molecule (e.g., a macrocyclic small molecule), a polymer, a nucleic acid (e.g., a DNA or RNA oligonucleotide), a peptide, a polypeptide, an oligosaccharide, an organometallic, a degrader, a macrocycle, or a protein, such as a mutated protein. The organic moiety may be bound to an aziridine moiety as disclosed herein in a variety of ways, and persons of skill in the art are familiar with methodologies of installing an aziridine-containing synthetic intermediate described herein in a monovalent organic moiety. Non-limiting examples include the schemes below:

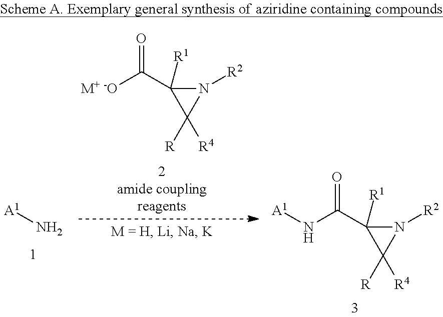

As shown in Scheme A, compounds of this type may be prepared by the reaction of an appropriate amine substituted monovalent organic moiety (1) with a carboxylate substituted aziridine (2) in the presence of standard amide coupling reagents to afford the final compound (3).

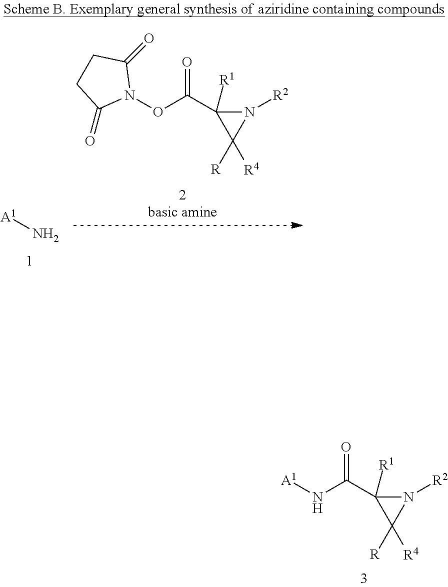

As shown in Scheme B, compounds of this type may be prepared by the reaction of an appropriate amine substituted monovalent organic moiety (1) with an aziridine containing an activated ester (2) in the presence of a basic amine to afford the final compound (3).

In some embodiments, a compound containing an aziridine moiety bound to a monovalent organic moiety may have the structure of Formula II:

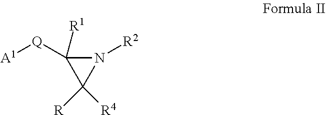

or a pharmaceutically acceptable salt thereof,
wherein A¹ is a monovalent organic moiety;
Q is

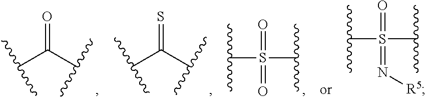

R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl or —Si(R^{1a})₃;
or R and R¹ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R and R² combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and R¹ and R⁴ are each, independently, hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a}); or
R and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R² combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and each of R and R⁴ is, independently, hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R² and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and each of R¹ and R is, independently, hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
each R^{1a}, R^{2a}, R^{2c}, R^{2d} and R^{2e} is, independently, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl;
R^{2b} is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁵ is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl,
and wherein each hydrogen is independently, optionally, isotopically enriched for deuterium.

In some embodiments, at least one of R and R⁴ is not hydrogen.

In some embodiments, the compound of Formula II, or a pharmaceutically acceptable salt thereof, has the structure of Formula IIa:

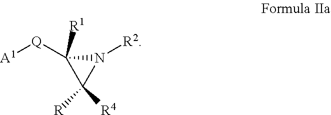

In some embodiments, the compound of Formula II, or a pharmaceutically acceptable salt thereof, has the structure of Formula IIb:

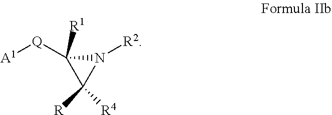

In some embodiments, the compound of Formula II, or a pharmaceutically acceptable salt thereof, has the structure of Formula IIc:

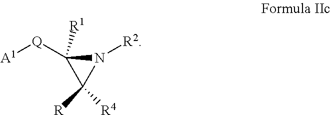

In some embodiments, the compound of Formula II, or a pharmaceutically acceptable salt thereof, has the structure of Formula IId:

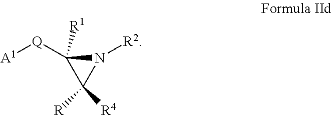

In some embodiments, Q is

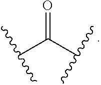 [SMILES: *C(*)=O]

In some embodiments, an organic moiety may be bound to an aziridine in alternative ways, such as to R, R¹, R² or R⁴.

In some embodiments, A¹ is or comprises a peptide. In some embodiments, A¹ is or comprises a protein. In some embodiments, A¹ is or comprises a nucleic acid. In some embodiments, A¹ is a small molecule. In some embodiments, A¹ is or comprises a macrocyclic small molecule.

In some embodiments, one or more compounds of WO 2023/141300 may be excluded from any embodiment herein. In some embodiments, one or more compounds of WO 2022/271658 may be excluded from any embodiment herein. In some embodiments, one or more compounds of WO 2023/208005 may be excluded from any embodiment herein.

In some embodiments, the compound is not a compound disclosed in WO 2021/091967. In some embodiments, the compound is not a compound of Table 2.

TABLE 2

| Ex# | Structure |
|---|---|
| C1 | 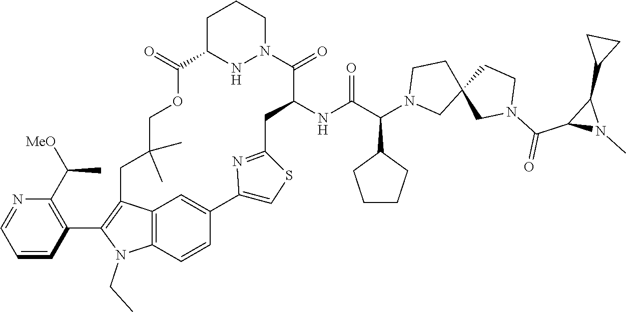 |
| C2 | 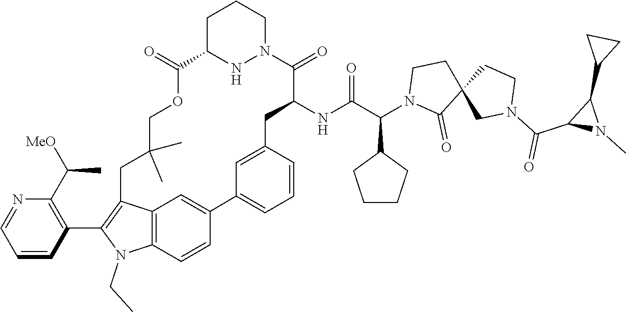 |
| C3 | 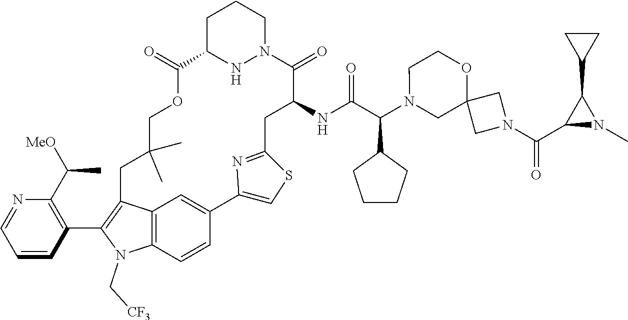 |
| C4 | 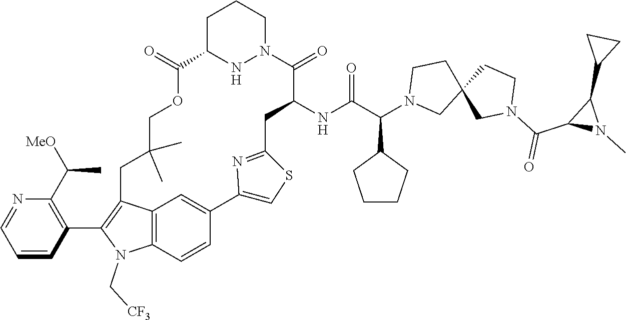 |
| C5 | 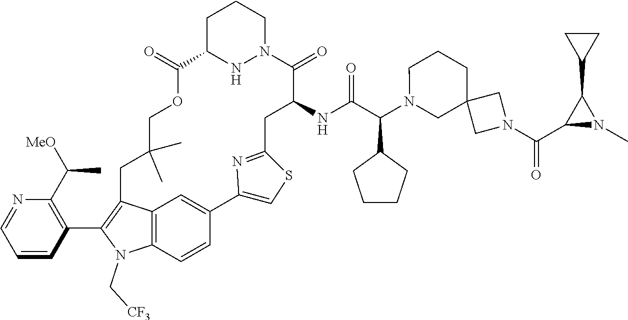 |
| C6 | 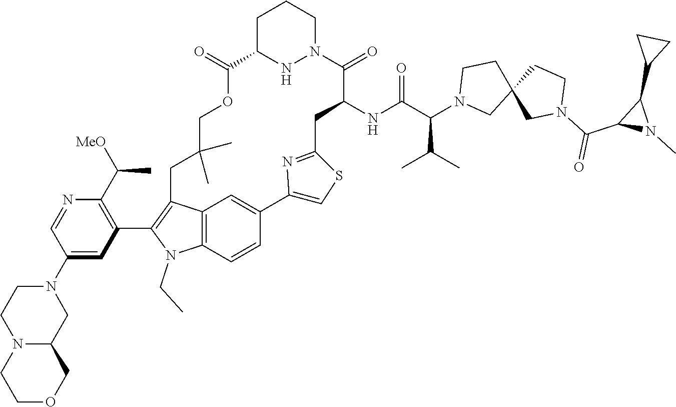 |
| C7 | 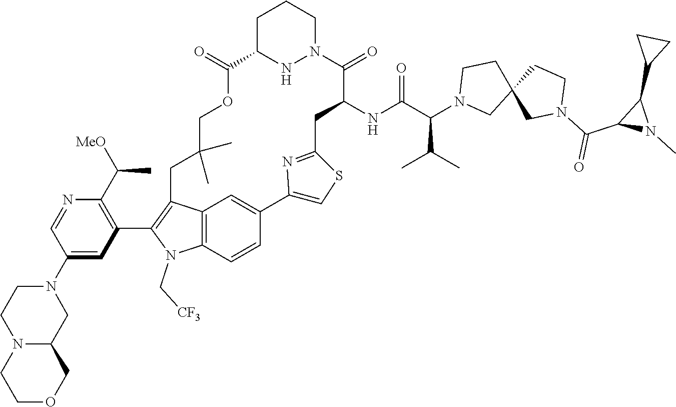 |
| C8 | 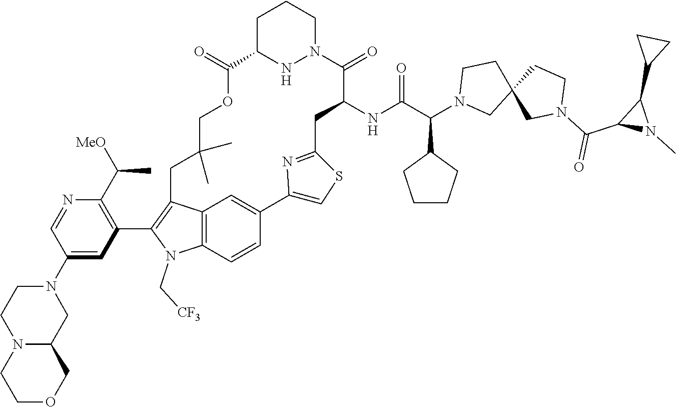 |
| C9 | 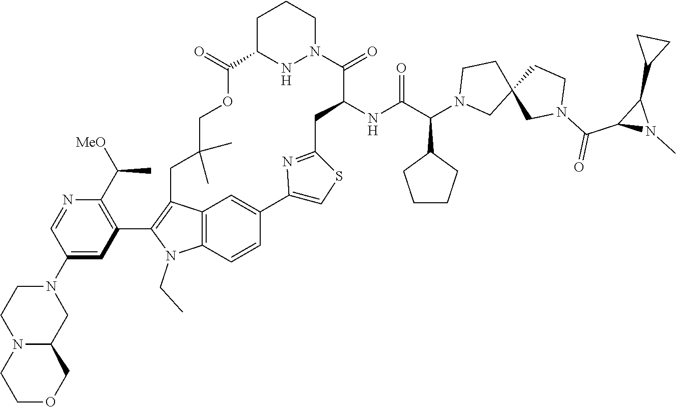 |
| C10 | 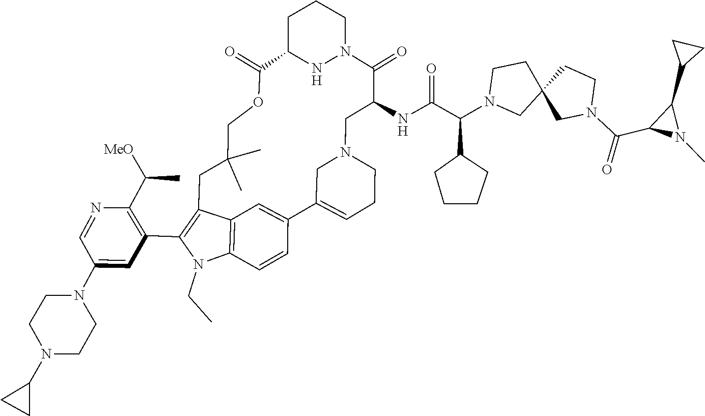 |
| C11 | 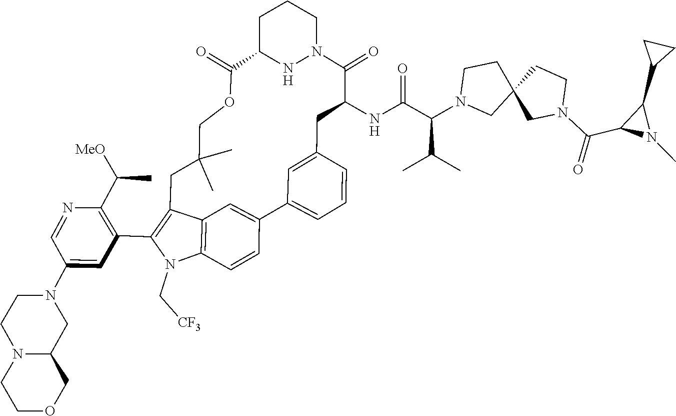 |
| C12 | 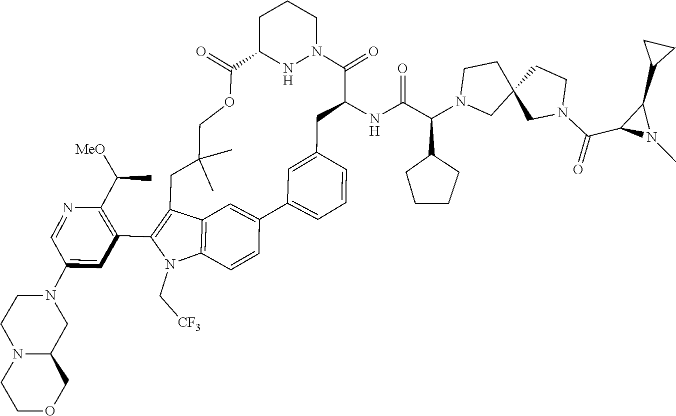 |
| C13 | 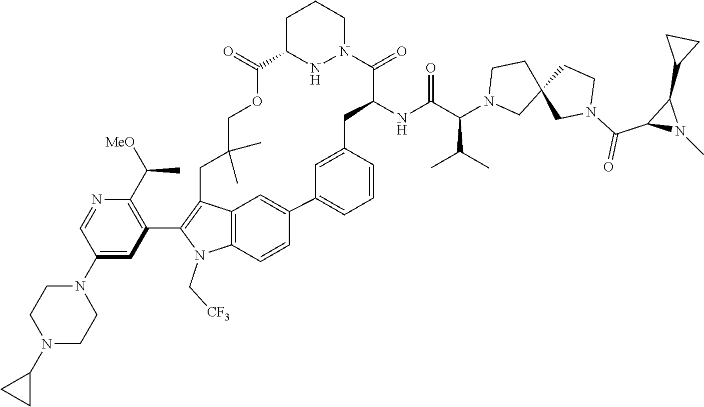 |
| C14 | 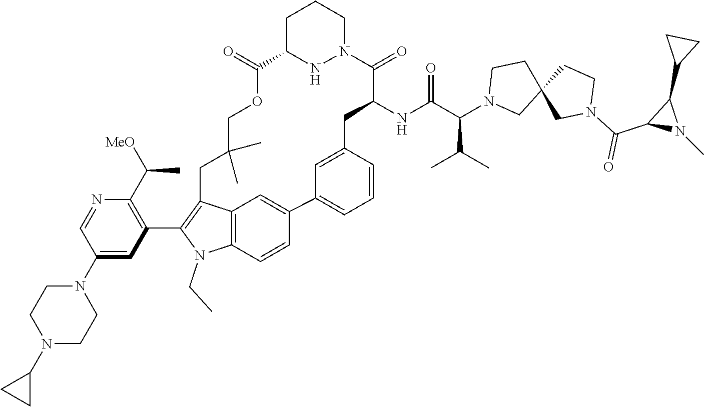 |
| C15 | 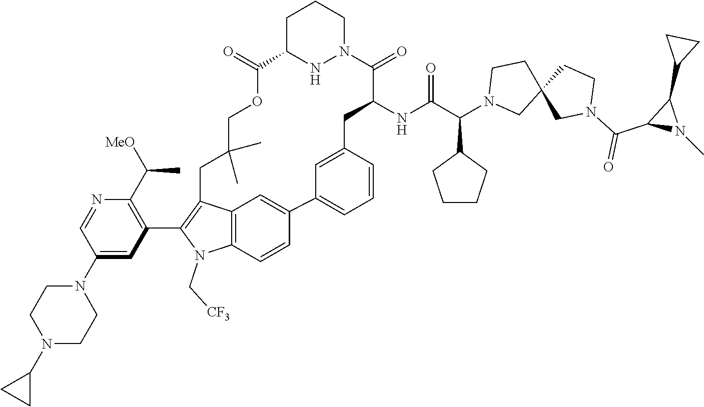 |
| C16 | 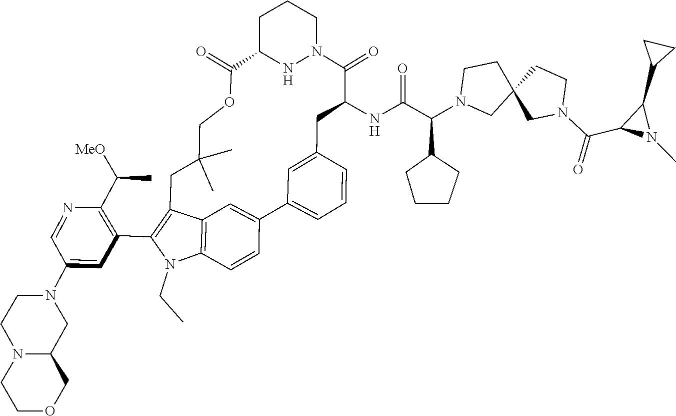 |
| C17 | 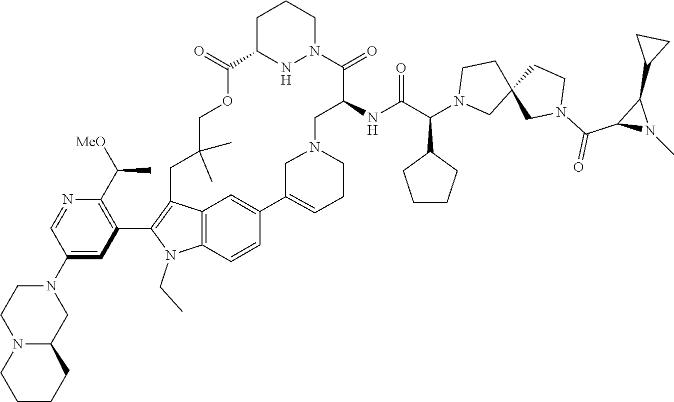 |
| C18 | 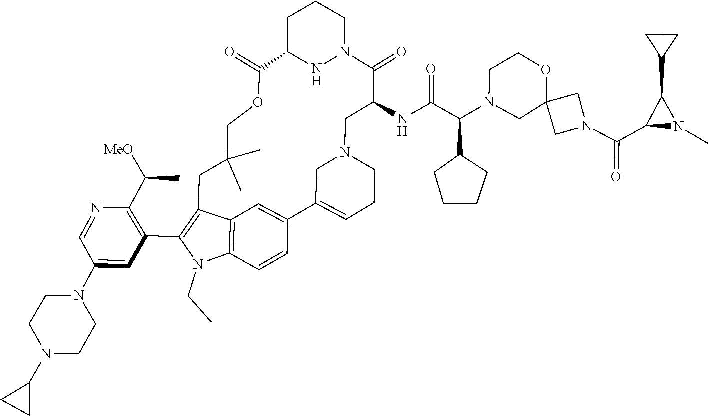 |
| C19 | 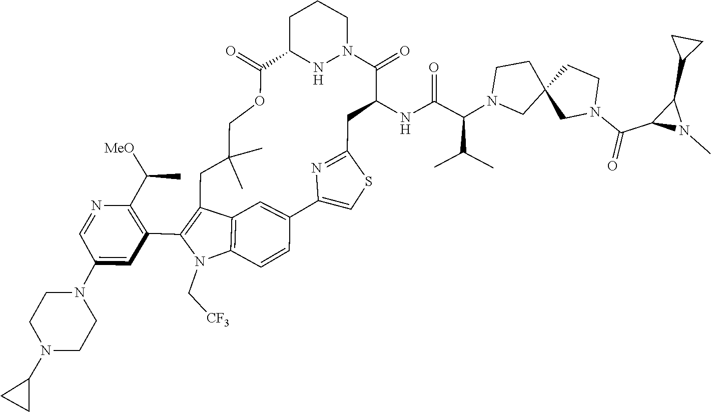 |
| C20 | 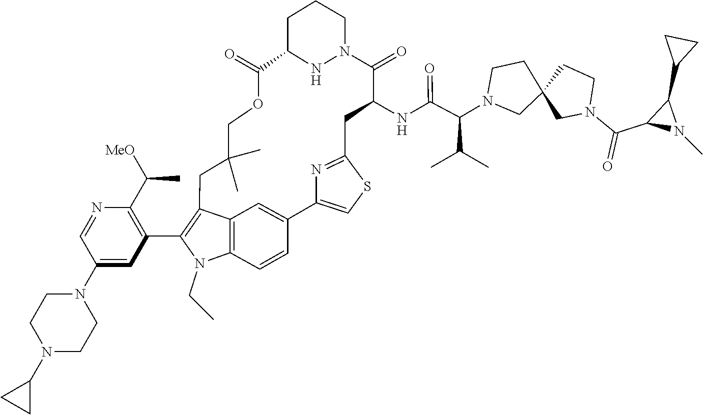 |
| C21 | 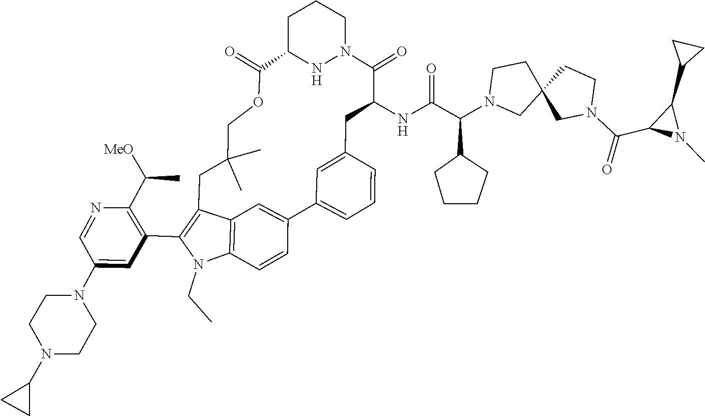 |
| C22 | 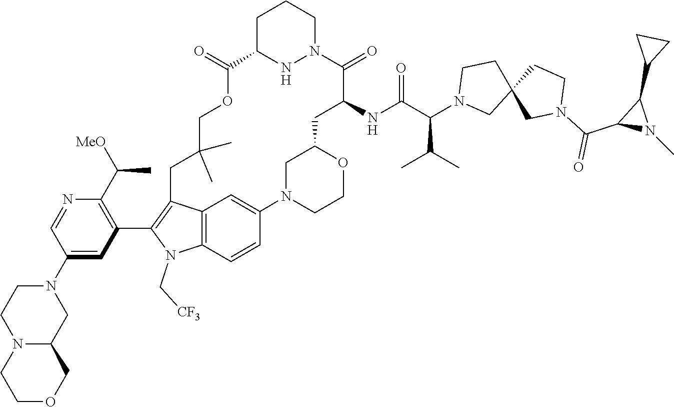 |
| C23 | 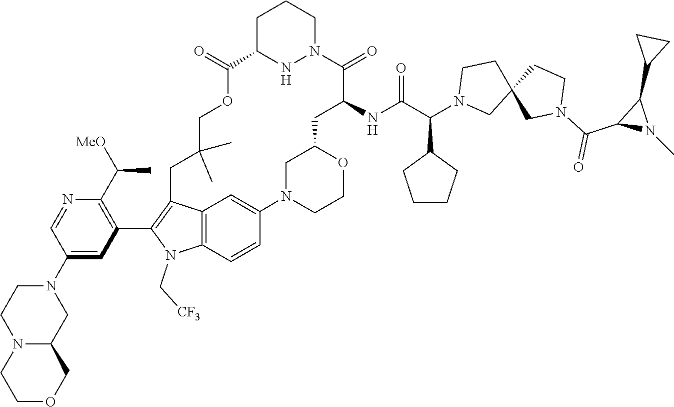 |
| C24 | 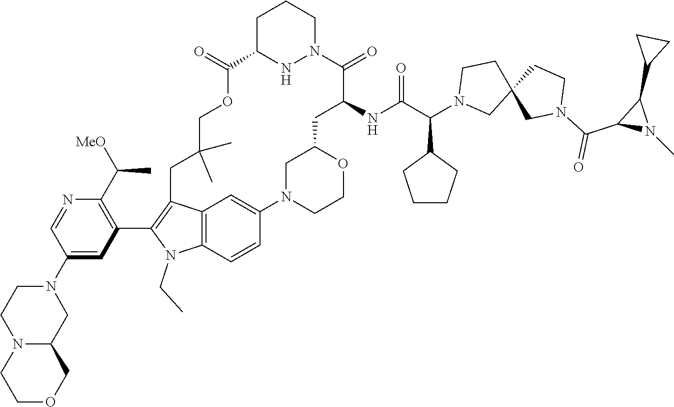 |
| C25 | 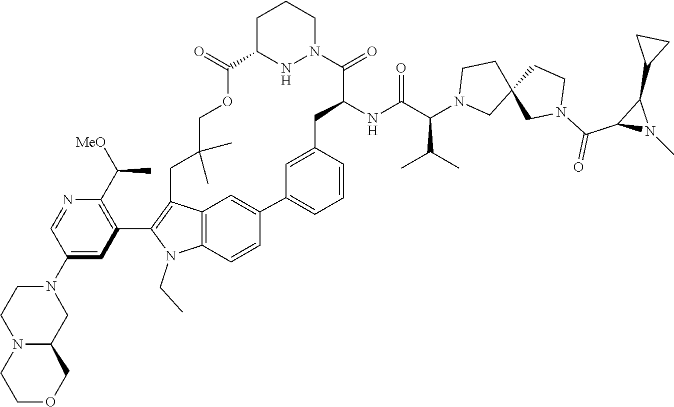 |
| C26 | 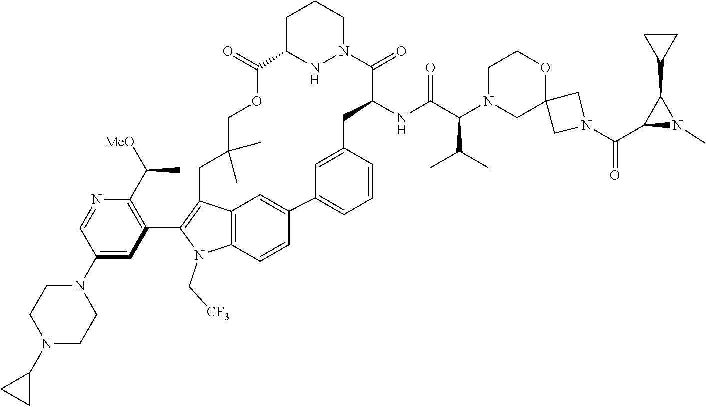 |
| C27 | 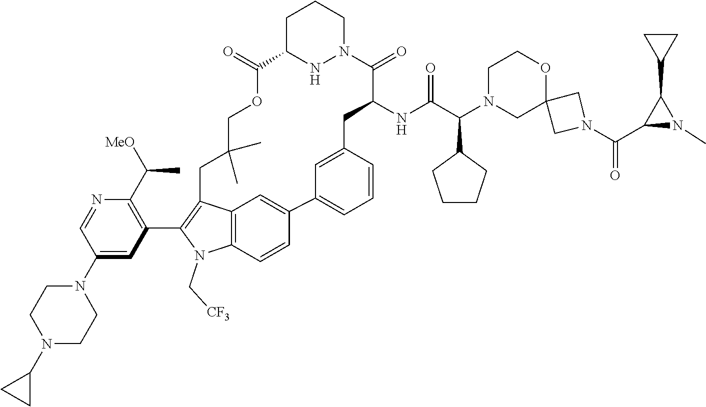 |
| C28 | 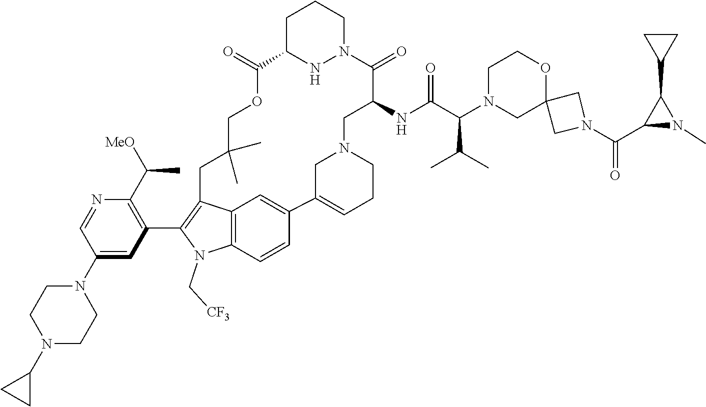 |
| C29 | 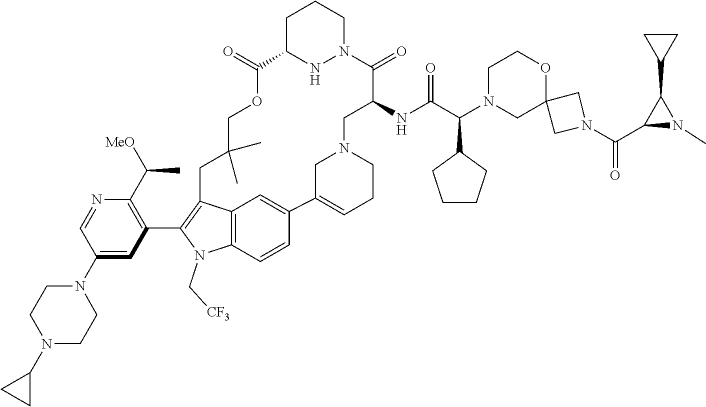 |
| C30 | 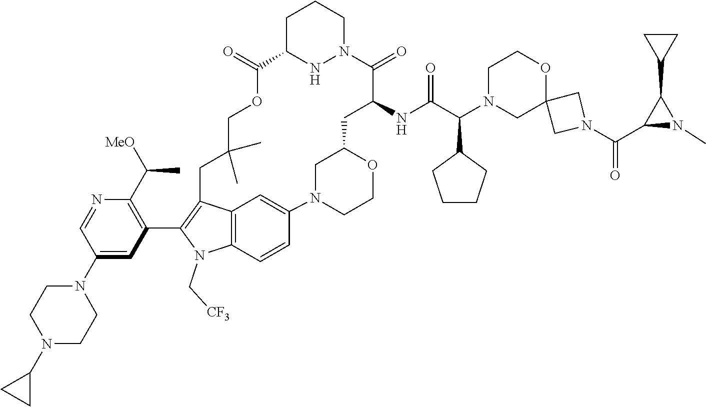 |
| C31 | 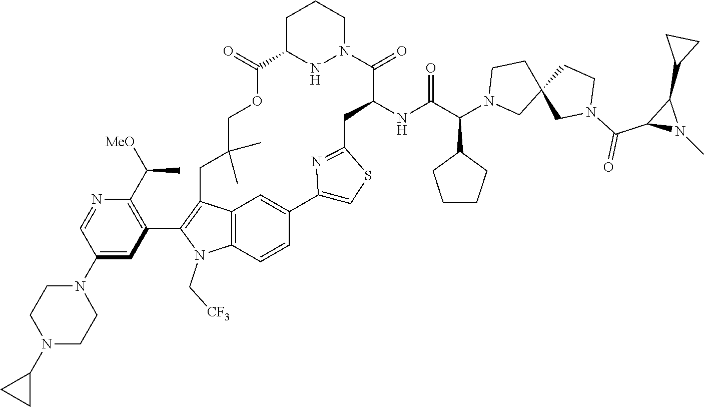 |
| C32 | 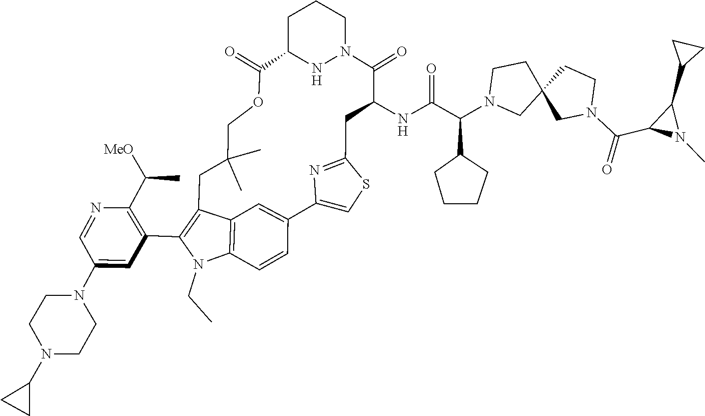 |
| C33 | 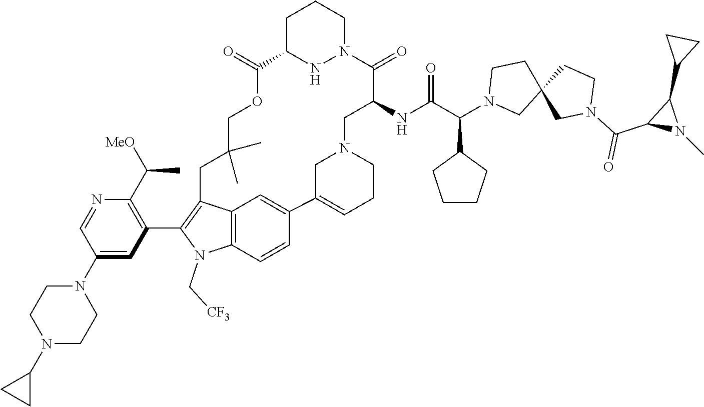 |
| C34 | 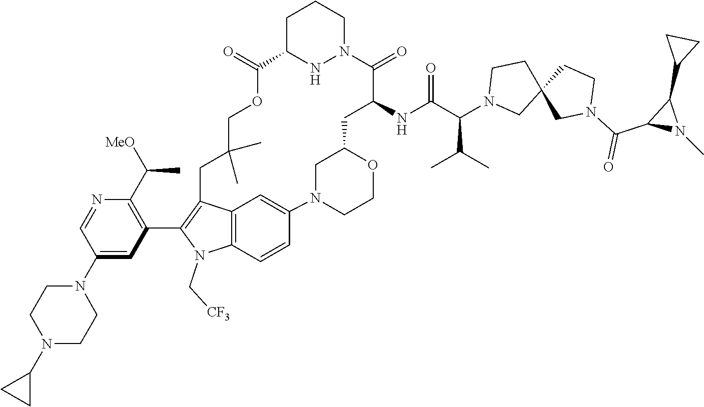 |
| C35 | 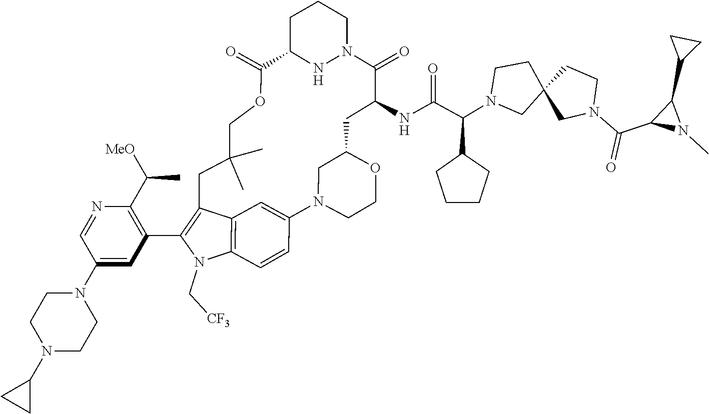 |
| C36 | 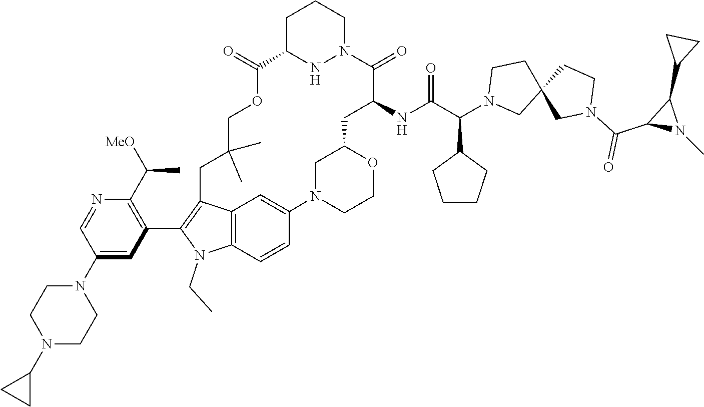 |
| C37 | 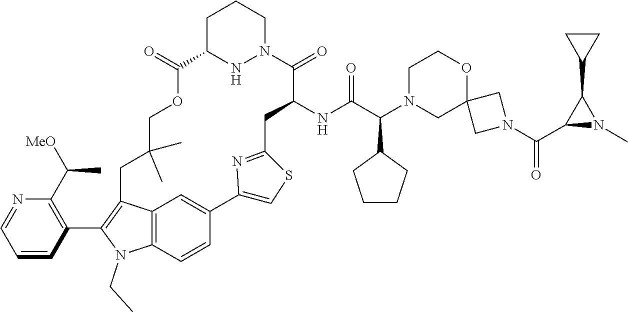 |
| C38 | 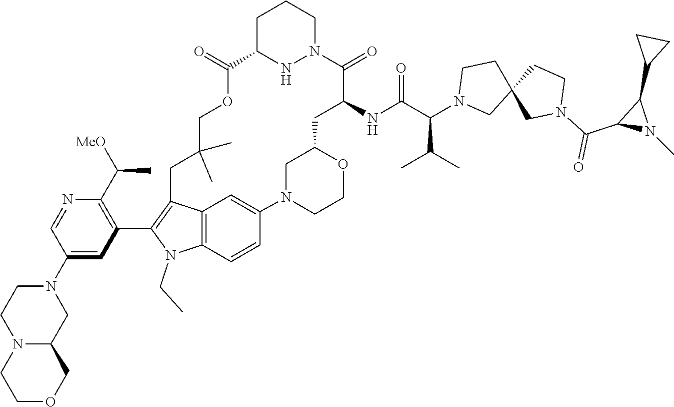 |
| C39 | 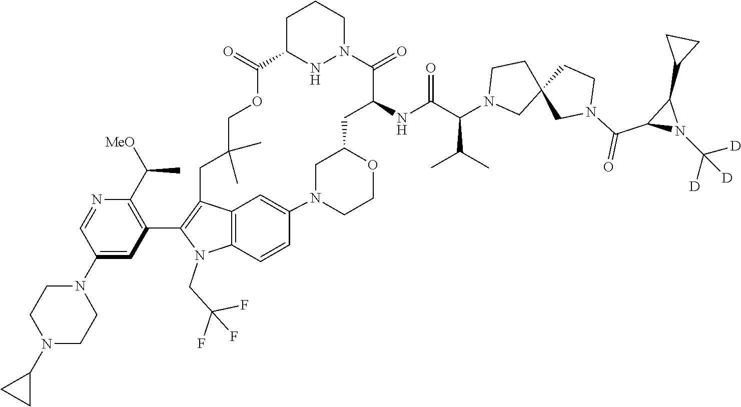 |
| C40 | 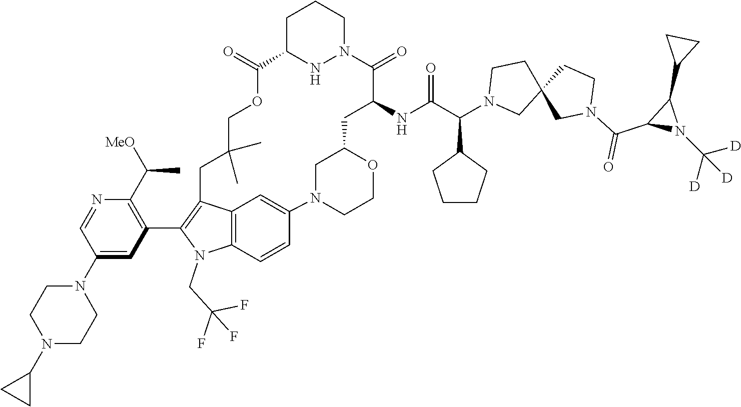 |
| C41 | 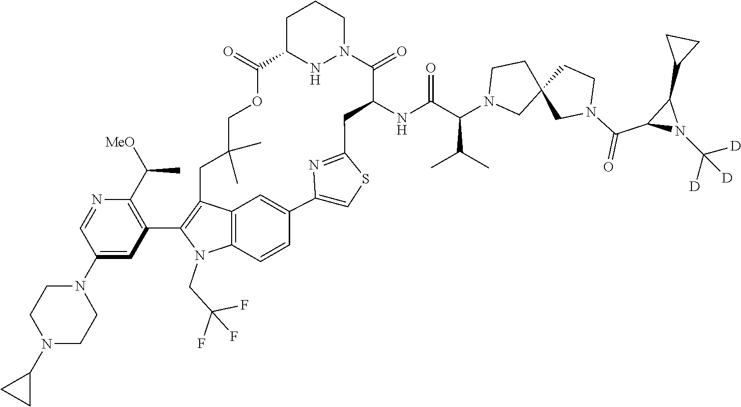 |
| C42 | 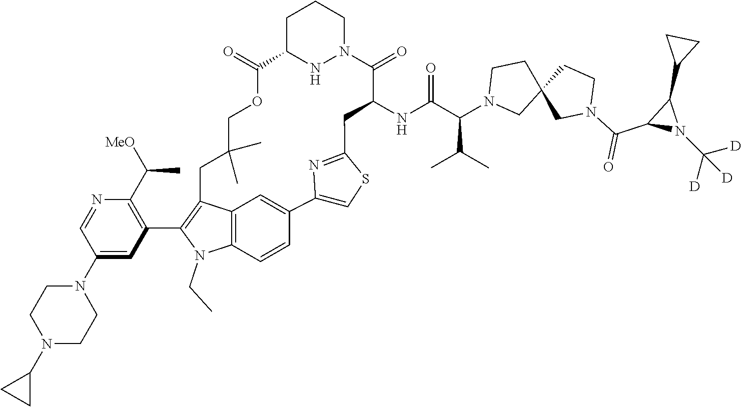 |
| C43 | 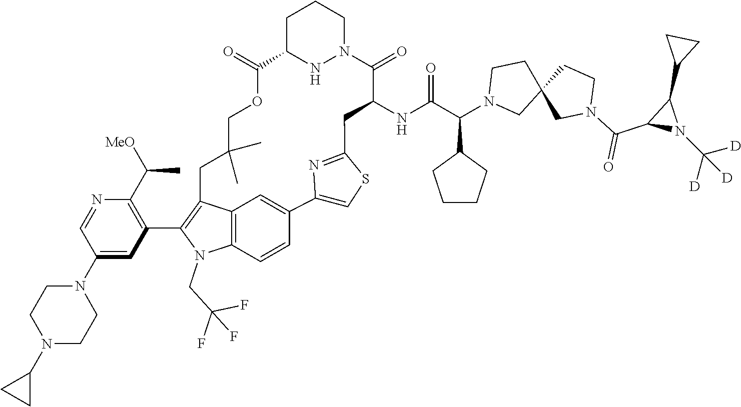 |
| C44 | 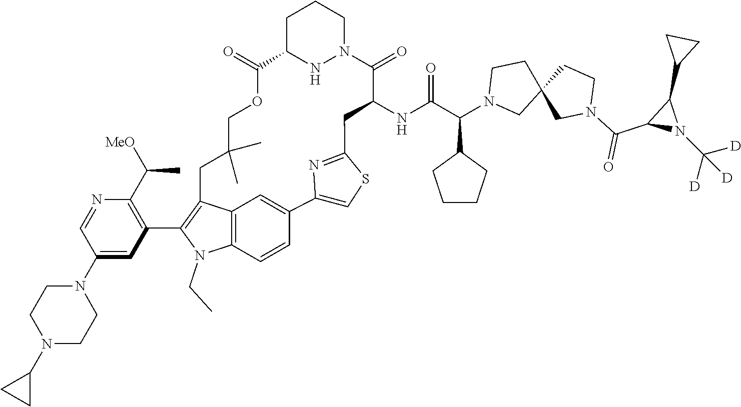 |
| C45 | 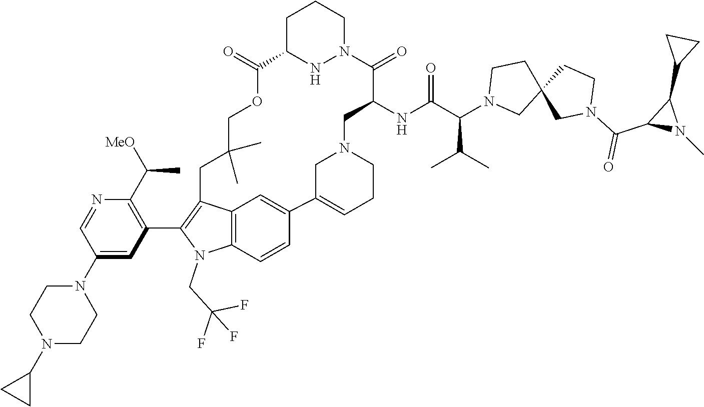 |
| C46 | 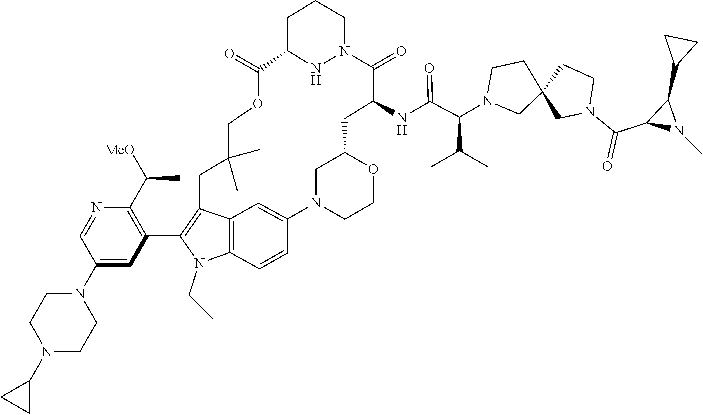 |
| C47 | 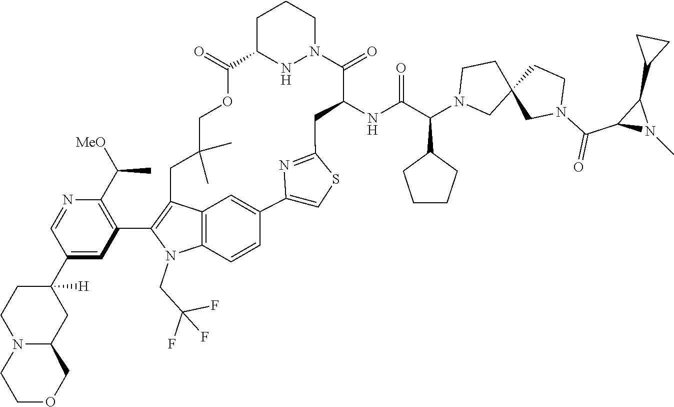 |
| C48 | 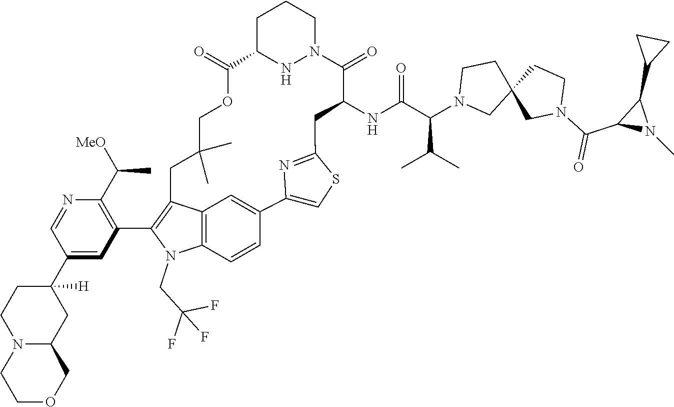 |
| C49 | 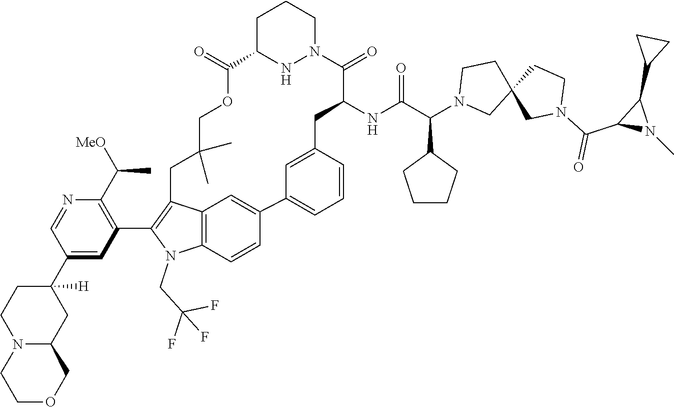 |
| C50 | 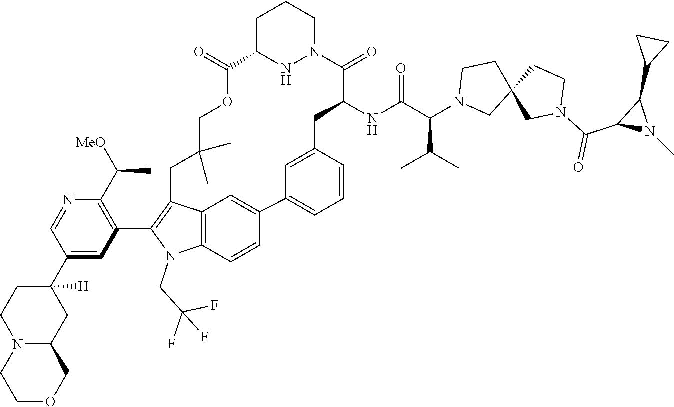 |
| C51 | 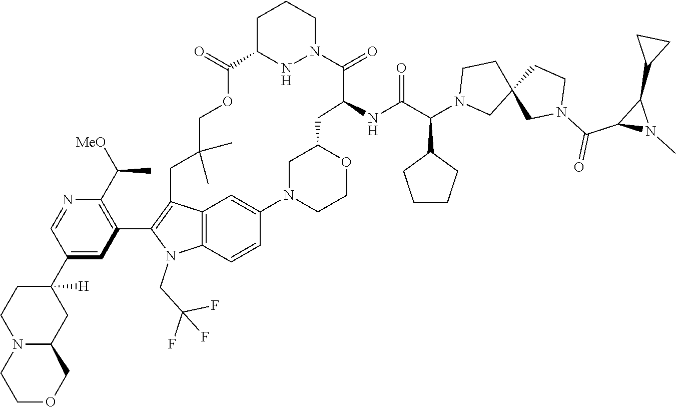 |
| C52 | 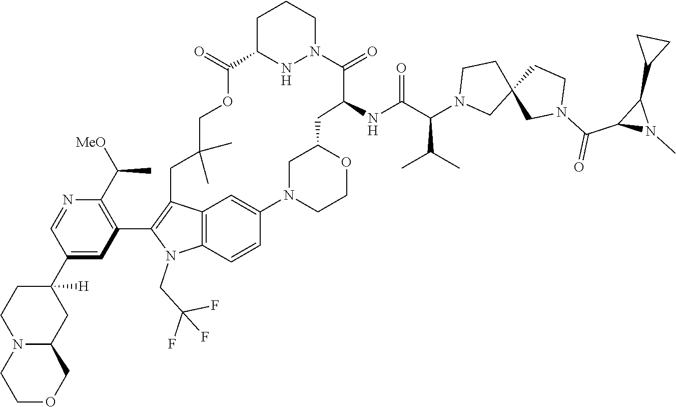 |
| C53 | 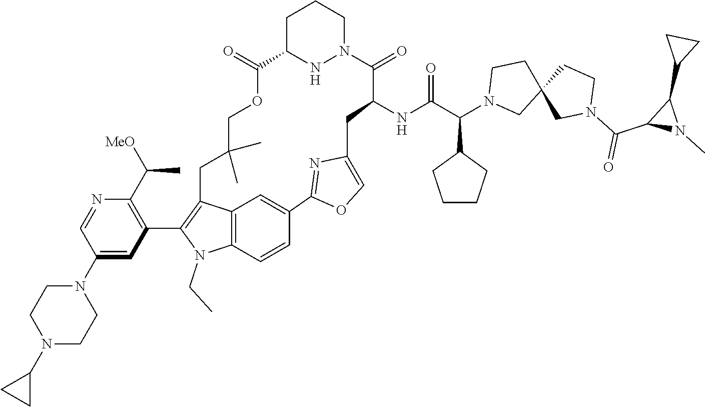 |
| C54 | 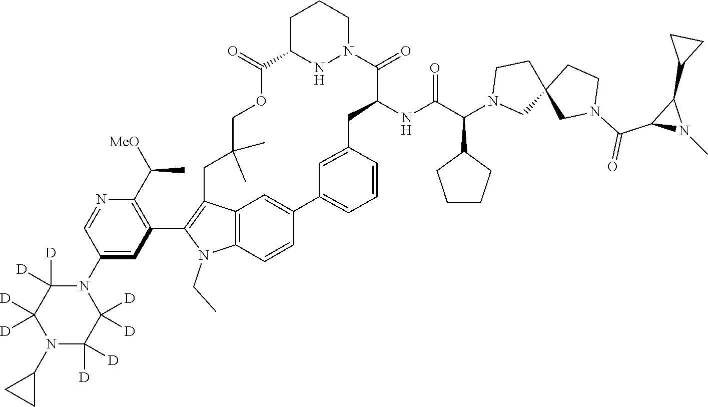 |
| C55 | 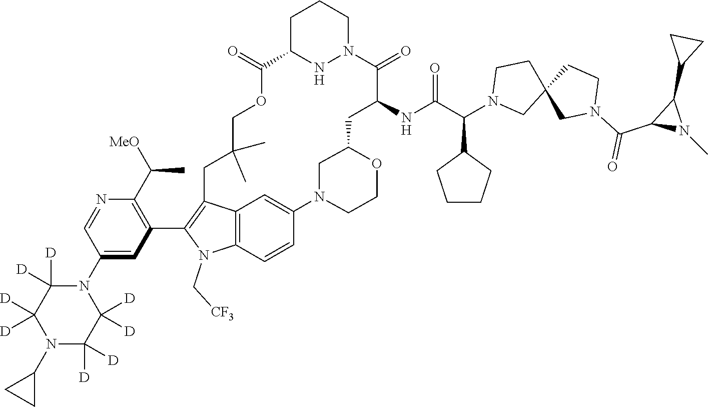 |
| C56 | 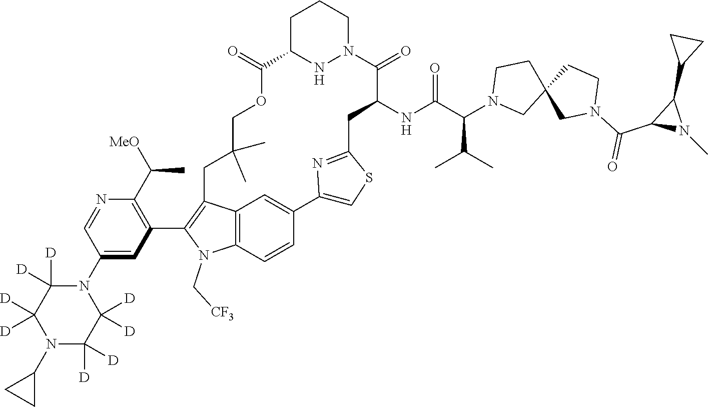 |
| C57 | 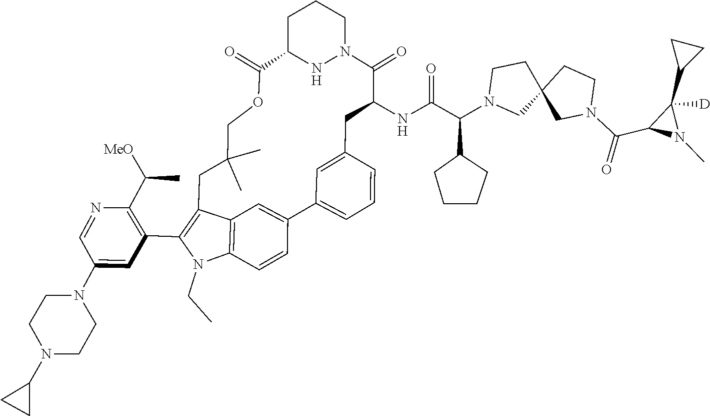 |
| C58 | 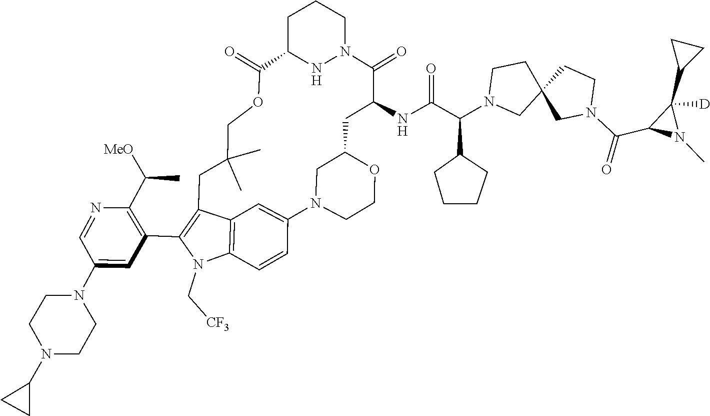 |
| C59 | 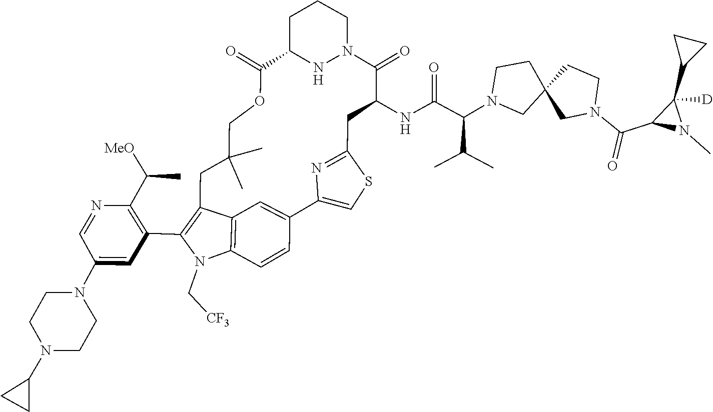 |
| C60 | 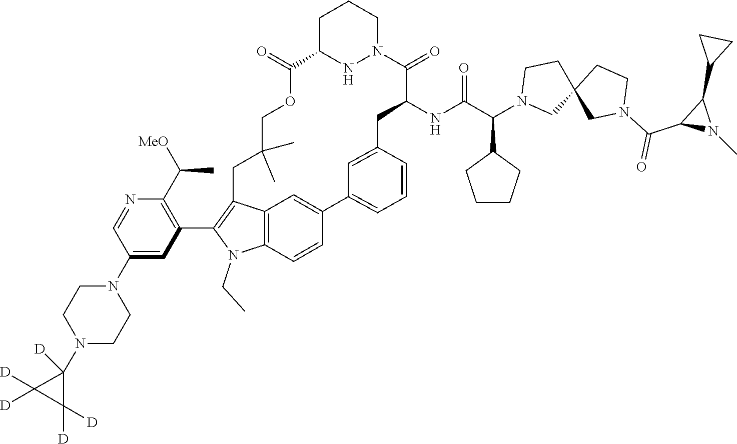 |
| C61 | 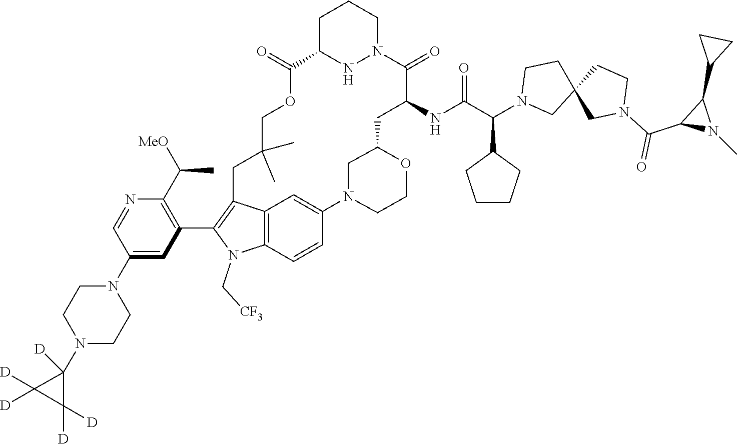 |
| C62 | 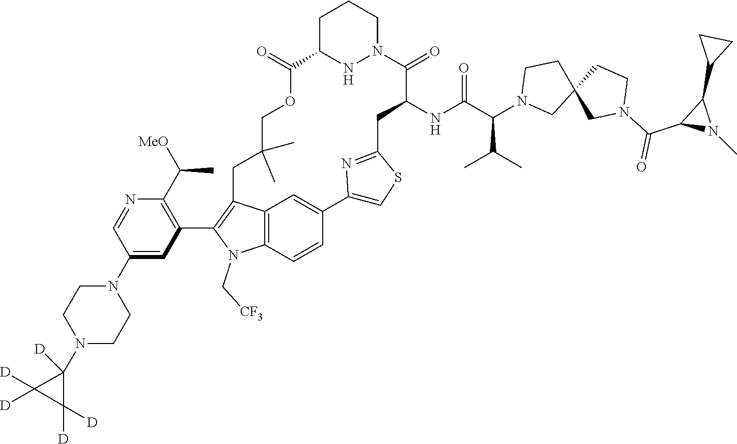 |
| C63 | 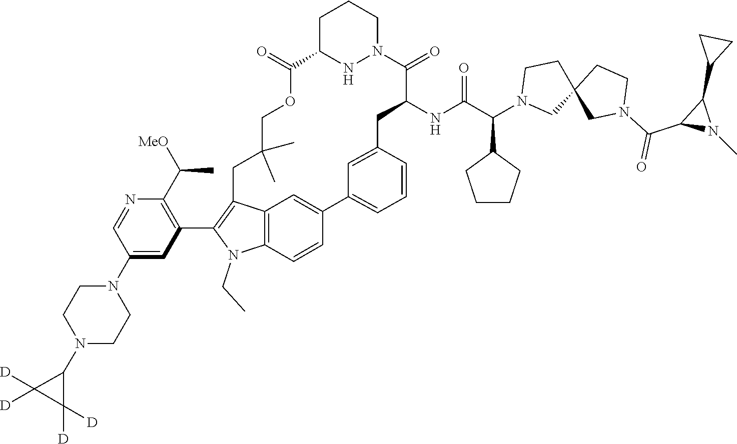 |
| C64 | 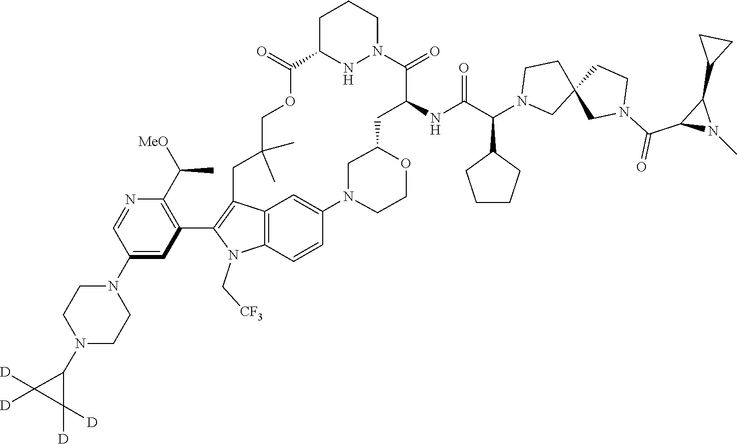 |
| C65 | 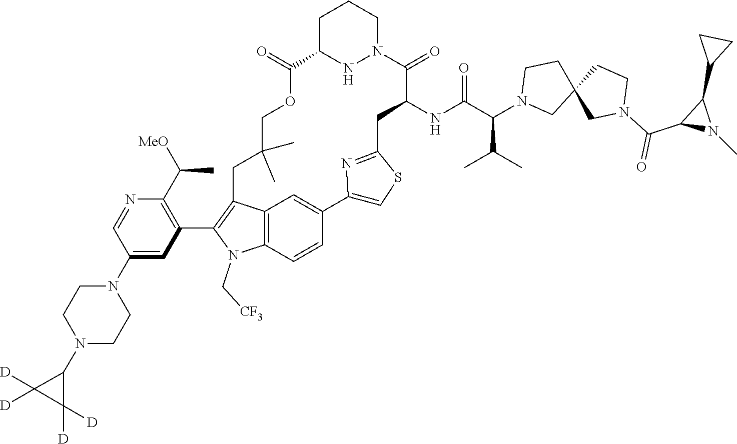 |
| C66 | 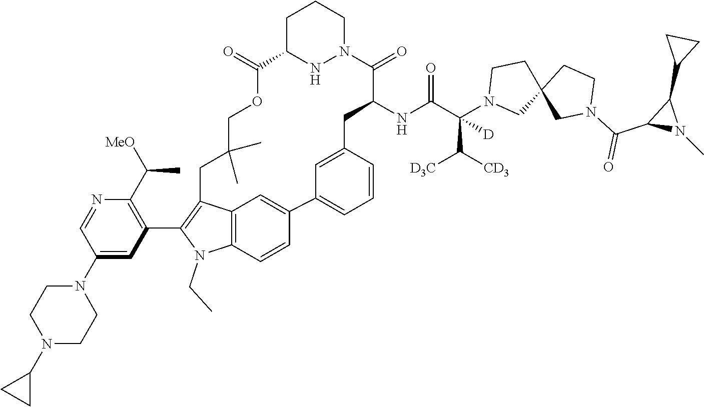 |
| C67 | 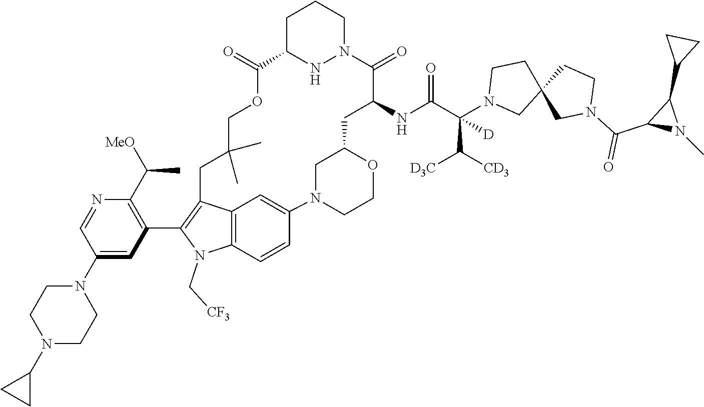 |
| C68 | 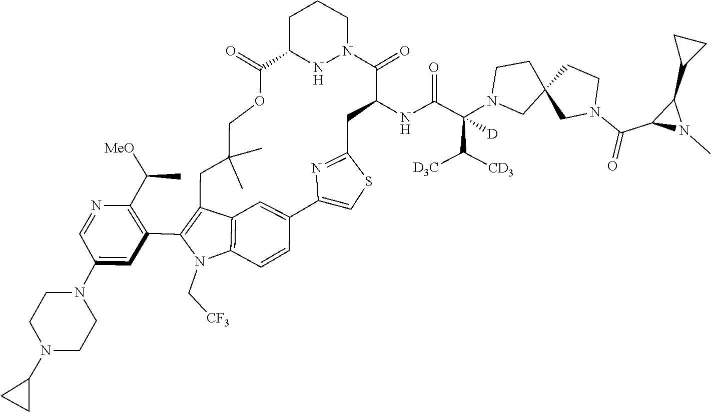 |
| C69 | 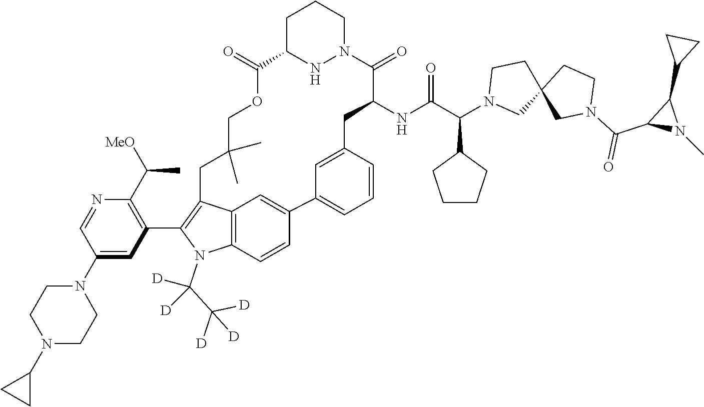 |
| C70 | 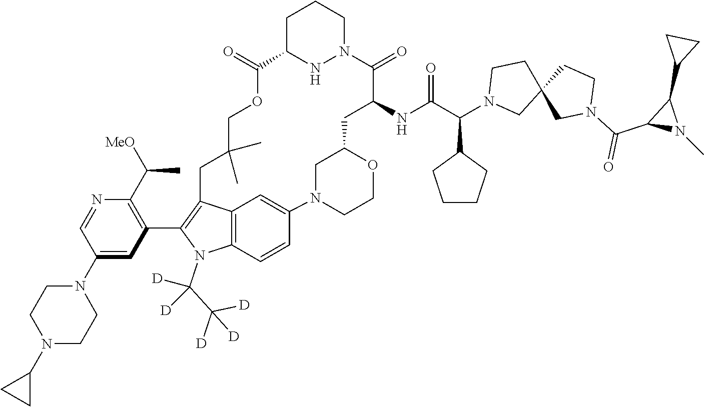 |
| C71 | 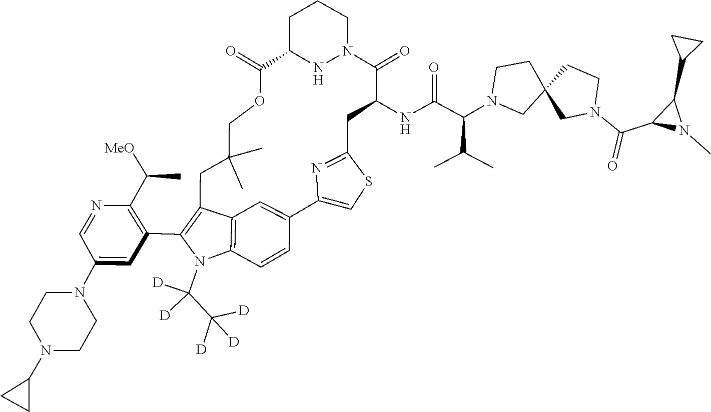 |
| C72 | 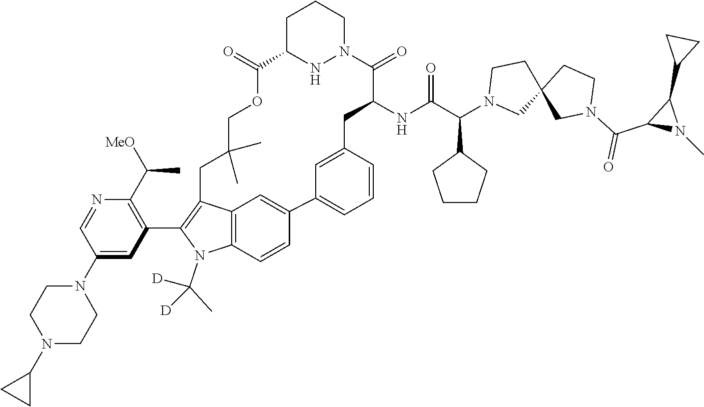 |
| C73 | 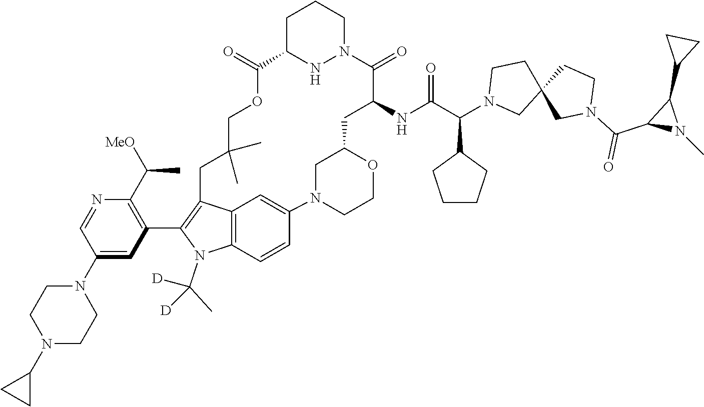 |
| C74 | 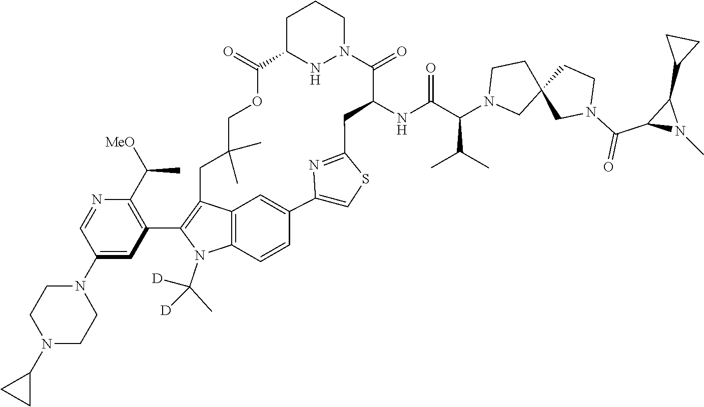 |
| C75 | 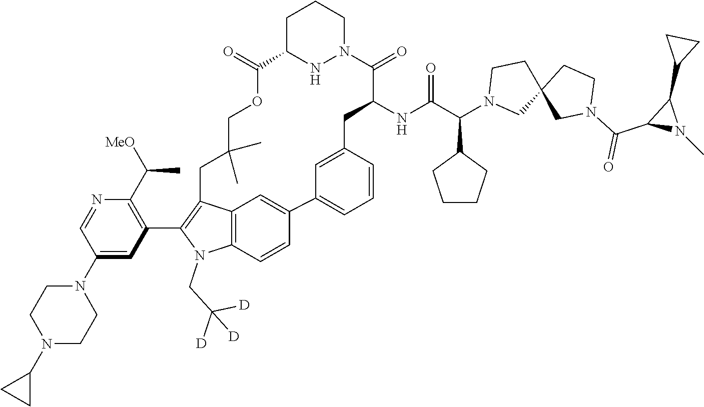 |
| C76 | 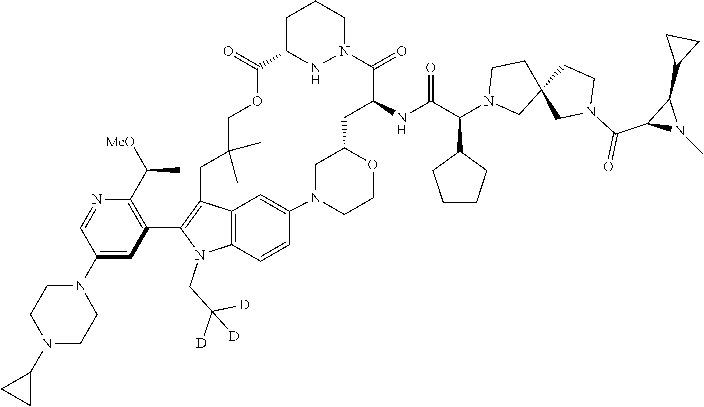 |
| C77 | 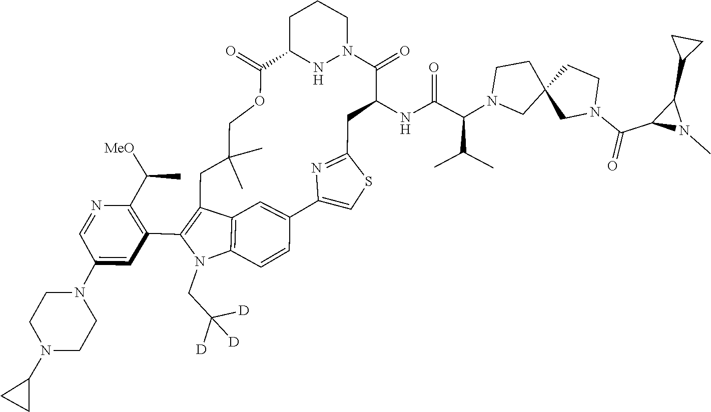 |
| C78 | 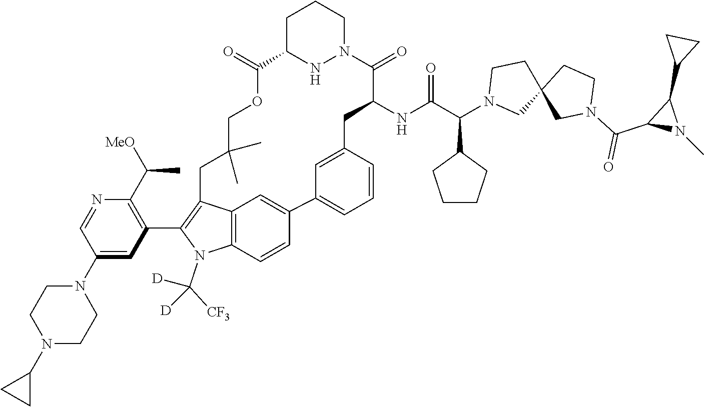 |
| C79 | 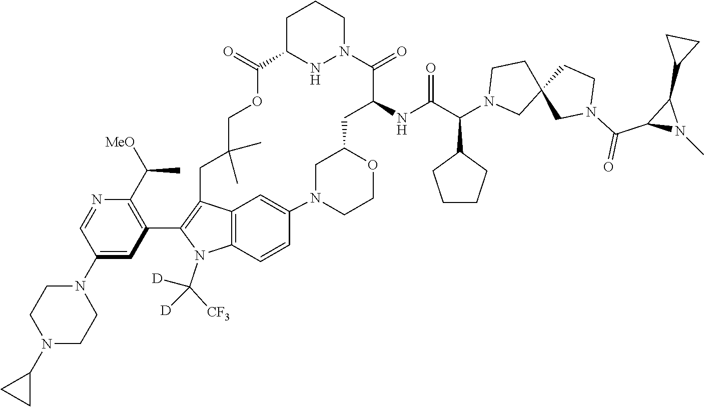 |
| C80 | 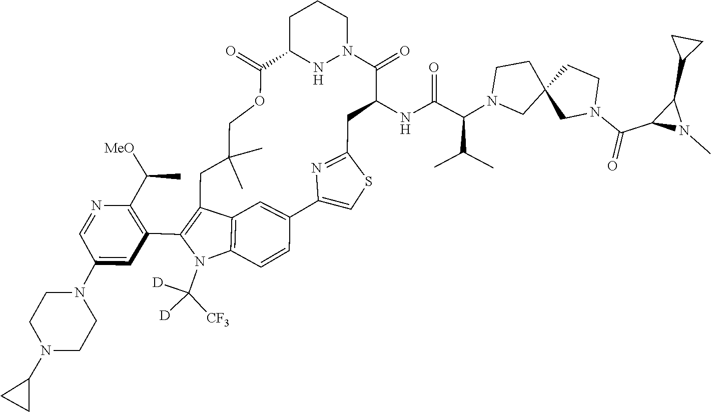 |
| C81 | 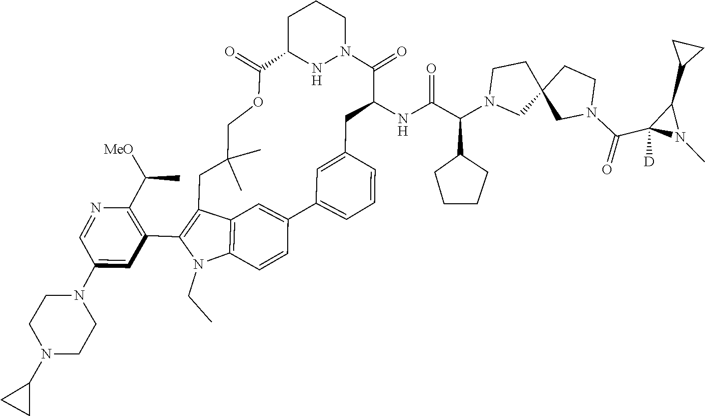 |
| C82 | 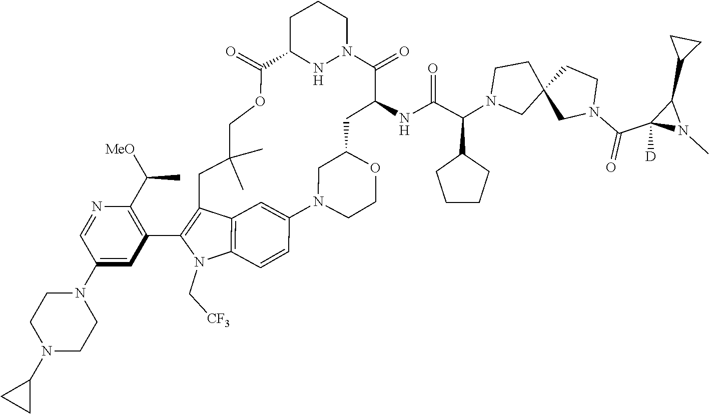 |
| C83 | 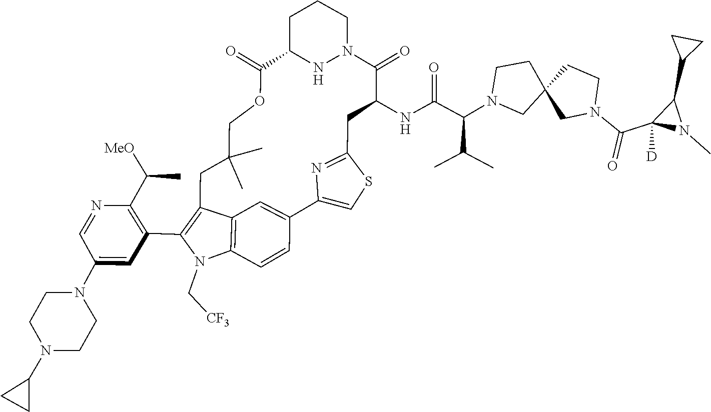 |

### Presenter Protein Binding Moiety

In some embodiments of the compounds described above, A¹ is a presenter protein binding moiety. This moiety may include a group of ring atoms (e.g., 5 to 20 ring atoms, 5 to 10 ring atoms, 10 to 20 ring atoms) and the moieties attached thereto (e.g., atoms within 20 atoms of a ring atom such as, atoms within 15 atoms of a ring atom, atoms within 10 atoms of a ring atom, atoms within 5 atoms of a ring atom) that participate in binding to a presenter protein such that a provided compound specifically binds to said presenter protein, for example, with a K_{D} of less than 10 μM (e.g., less than 5 μM, less than 1 μM, less than 500 nM, less than 200 nM, less than 100 nM, less than 75 nM, less than 50 nM, less than 25 nM, less than 10 nM) or inhibits the peptidyl-prolyl isomerase activity of the presenter protein, for example, with an IC₅₀ of less than 1 μM (e.g., less than 0.5 μM, less than 0.1 μM, less than 0.05 μM, less than 0.01 μM). In some embodiments, the presenter protein binding moiety does not encompass the entirety of atoms in a provided compound that interact with the presenter protein. In some embodiments, one or more atoms of the presenter protein binding moiety may be within the target protein interaction moiety (e.g., eukaryotic target protein interacting moiety such as a mammalian target protein interacting moiety or a fungal target protein interacting moiety or prokaryotic target protein interacting moiety such as a bacterial target protein interacting moiety). In certain embodiments, one or more atoms of the presenter protein binding moiety do not interact with the presenter protein.

In some embodiments, a presenter protein binding moiety includes a N-acyl proline moiety, an N-acyl-pipecolic acid moiety, an N-acyl 3-morpholino-carboxylic acid moiety, and/or an N-acyl piperazic acid moiety (e.g., with acylation on either nitrogen atom. In certain embodiments, a presenter protein binding moiety includes an N-acyl-pipecolic acid moiety. In some embodiments, a presenter protein binding moiety includes an N-acyl proline moiety. In certain embodiments, a presenter protein binding moiety includes an N-acyl 3-morpholino-carboxylic acid moiety. In some embodiments, a presenter protein binding moiety includes a N-acyl piperazic acid moiety.

In some embodiments, at least one atom of a presenter protein binding moiety participates in binding with one or more (e.g., two, three, four, five, six, seven, eight, nine, ten, eleven, twelve, thirteen, fourteen, or fifteen) of Tyr 27, Phe 37, Asp 38, Arg 41, Phe 47, Gln 54, Glu 55, Val 56, Ile 57, Trp 60, Ala 82, Try 83, His 88, Ile 92, and/or Phe 100 of FKBP12. In some embodiments, at least one at of a presenter protein binding moiety participates in binding with at least one (e.g., two, three, or four) of Arg 41, Gln 54, Glu 55, and/or Ala 82 of FKBP12.

In some embodiments, the presenter protein binding moiety has the structure of Formula IV:

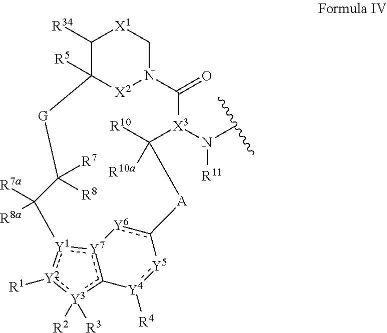

wherein the dotted lines represent zero, one, two, three, or four non-adjacent double bonds; A is —N(H or CH₃)C(O)—(CH₂)—where the amino nitrogen is bound to the carbon atom of —CH(R¹⁰)—, optionally substituted C₂-C₄ alkylene, optionally substituted C₁-C₄ heteroalkylene, or optionally substituted C₂-C₄ alkenylene, optionally substituted 3- to 6-membered cycloalkylene, optionally substituted 3- to 6-membered heterocycloalkylene, optionally substituted 6-membered arylene, or optionally substituted 5- to 10-membered heteroarylene;
X¹ is optionally substituted C₁-C₂ alkylene, NR, O, or S(O)ₙ;
X² is O or NH;
X³ is N or CH;
   n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂; each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂ or N;
Y⁶ is C(O), CH, CH₂, or N;
R¹ is cyano, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6 to 10-membered aryl, or optionally substituted 5- to 10-membered heteroaryl, or
R¹ and R² combine with the atoms to which they are attached to form an optionally substituted 3- to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl; R³ is absent, or
R² and R³ combine with the atom to which they are attached to form an optionally substituted 3- to 8-membered cycloalkyl or optionally substituted 3- to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or R⁶ and R⁷ combine with the carbon atoms to which they are attached to form an optionally substituted 3- to 6-membered cycloalkyl or optionally substituted 3- to 7-membered heterocycloalkyl; R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6 to 10-membered aryl, or R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3- to 6-membered cycloalkyl, or optionally substituted 3- to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6 to 10-membered aryl, or R^{7′} and R^{8′} combine with the carbon atom to which they are attached to form optionally substituted 3- to 6-membered cycloalkyl or optionally substituted 3- to 7-membered heterocycloalkyl;
R⁹ is hydrogen, F, optionally substituted C₁-C₆ alkyl, optionally substituted C₁-C₆ heteroalkyl, optionally substituted 3- to 6-membered cycloalkyl, or optionally substituted 3- to 7-membered heterocycloalkyl, or
R⁹ and L combine with the atoms to which they are attached to form an optionally substituted 3- to 14-membered heterocycloalkyl; R^{9′} is hydrogen or optionally substituted C₁-C₆ alkyl;
R¹⁰ is hydrogen, halo, hydroxy, C₁-C₃ alkoxy, or C₁-C₃ alkyl; R^{10a} is hydrogen or halo;
R¹¹ is hydrogen or C₁-C₃ alkyl; and
R³⁴ is hydrogen or C₁-C₃ alkyl. Each hydrogen of a compound of Formula IV is optionally isotopically enriched in deuterium.

In some embodiments, the presenter protein binding moiety has the structure of Formula V:

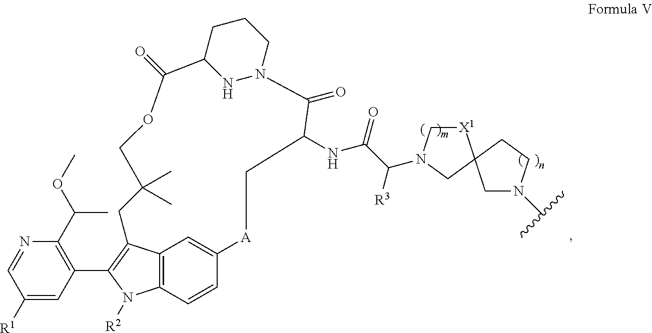

wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
X¹ is CH₂ or O;
m is 1 or 2;
n is 0 or 1;
R¹ is hydrogen or optionally substituted 3- to 10-membered heterocycloalkyl;
R² is optionally substituted C₁-C₆ alkyl; and
R³ is optionally substituted C₁-C₆ alkyl or optionally substituted 3- to 6-membered cycloalkyl.

In some embodiments, the presenter protein binding moiety has the structure of Formula VI:

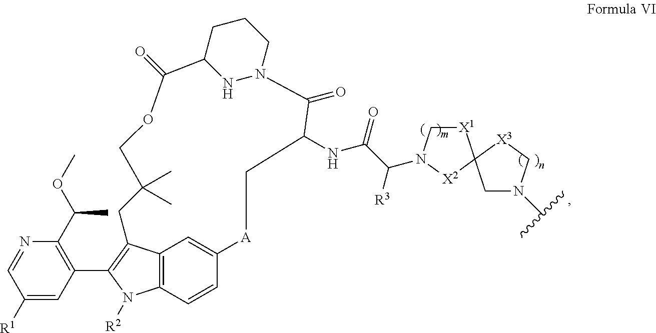

wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
X¹, X², and X³ are each independently selected from CH₂, CHF, CF₂, C═O, or O;
m is 1 or 2;
n is 0 or 1;
R¹ is hydrogen, optionally substituted C₁-C₆ heteroalkyl, or optionally substituted 3- to 10-membered heterocycloalkyl;
R² is optionally substituted C₁-C₆ alkyl; and
R³ is optionally substituted C₁-C₆ alkyl, optionally substituted C₁-C₆ heteroalkyl, optionally substituted 3- to 6-membered cycloalkyl, or optionally substituted heterocycloalkyl,
and wherein each hydrogen is independently, optionally, isotopically enriched for deuterium.

In some embodiments, the presenter protein binding moiety has the structure of any one of Formula VII, VIII, and IX:

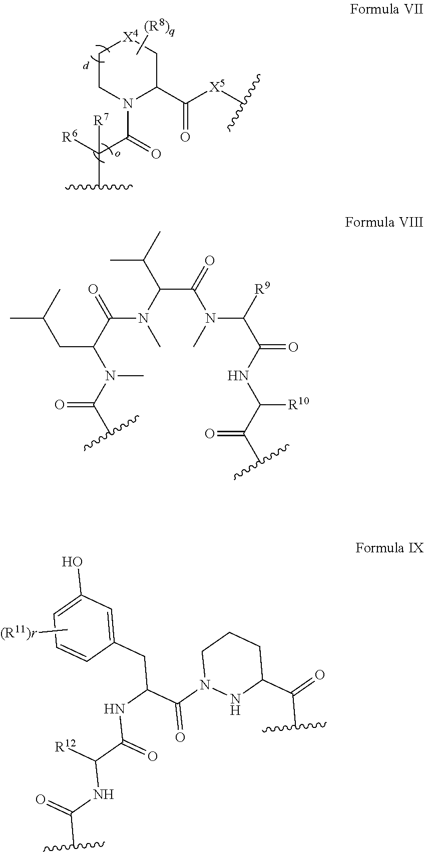

wherein o, and p are independently 0, 1, or 2;
q is an integer between 0 and 7;
r is an integer between 0 and 4;
X⁴ and X⁵ are each, independently, absent, CH₂, O, S, SO, SO₂, or NR11;
each R⁶ and R⁷ are independently hydrogen, hydroxyl, optionally substituted amino, halogen, thiol, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted C₃-C₁₀ carbocyclyl, optionally substituted C₆-C₁₀ aryl, optionally substituted C₆-C₁₀ aryl C₁-C₆ alkyl, optionally substituted C₂-C₉ heteroaryl, optionally substituted C₂-C₉ heteroaryl C₁-C₆ alkyl, optionally substituted C₂-C₉ heterocyclyl, optionally substituted C₂-C₉ heterocyclyl C₁-C₆ alkyl, or R⁶ and R⁷ combine with the carbon atom to which they are bound to form C═O;
each R⁸ is, independently, hydroxyl, optionally substituted amino, halogen, thiol, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted C₃-C₁₀ carbocyclyl, optionally substituted C₆-C₁₀ aryl, optionally substituted C₆-C₁₀ aryl C₁-C₆ alkyl, optionally substituted C₂-C₉ heteroaryl, optionally substituted C₂-C₉ heteroaryl C₁-C₆ alkyl, optionally substituted C₂-C₉ heterocyclyl, or optionally substituted C₂-C₉ heterocyclyl C₁-C₆ alkyl, or two R⁸ combine to form an optionally substituted C₃-C₁₀ carbocyclyl, optionally substituted C₆-C₁₀ aryl, or optionally substituted C₂-C₉ heteroaryl;
R⁹ is optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted C₃-C₁₀ carbocyclyl, optionally substituted C₆-C₁₀ aryl, optionally substituted C₆-C₁₀ aryl C₁-C₆ alkyl, optionally substituted C₂-C₉ heteroaryl, optionally substituted C₂-C₉ heteroaryl C₁-C₆ alkyl, optionally substituted C₂-C₉ heterocyclyl, or optionally substituted C₂-C₉ heterocyclyl C₁-C₆ alkyl;
R¹⁰ is optionally substituted C₁-C₆ alkyl;
each R¹¹ is, independently, hydroxyl, cyano, optionally substituted amino, halogen, thiol, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted C₃-C₁₀ carbocyclyl, optionally substituted C₆-C₁₀ aryl, optionally substituted C₆-C₁₀ aryl C₁-C₆ alkyl, optionally substituted C₂-C₉ heteroaryl, optionally substituted C₂-C₉ heteroaryl C₁-C₆ alkyl, optionally substituted C₂-C₉ heterocyclyl, or optionally substituted C₂-C₉ heterocyclyl C₁-C₆ alkyl; and
R¹² and R¹³ are each, independently, hydrogen, optionally substituted C₁-C₆ alkyl, optionally substituted C₂-C₆ alkenyl, optionally substituted C₂-C₆ alkynyl, optionally substituted aryl, C₃-C₇ carbocyclyl, optionally substituted C₆-C₁₀ aryl C₁-C₆ alkyl, and optionally substituted C₃-C₇ carbocyclyl C₁-C₆ alkyl.

In some embodiments, a presenter protein binding moiety has the structure of:

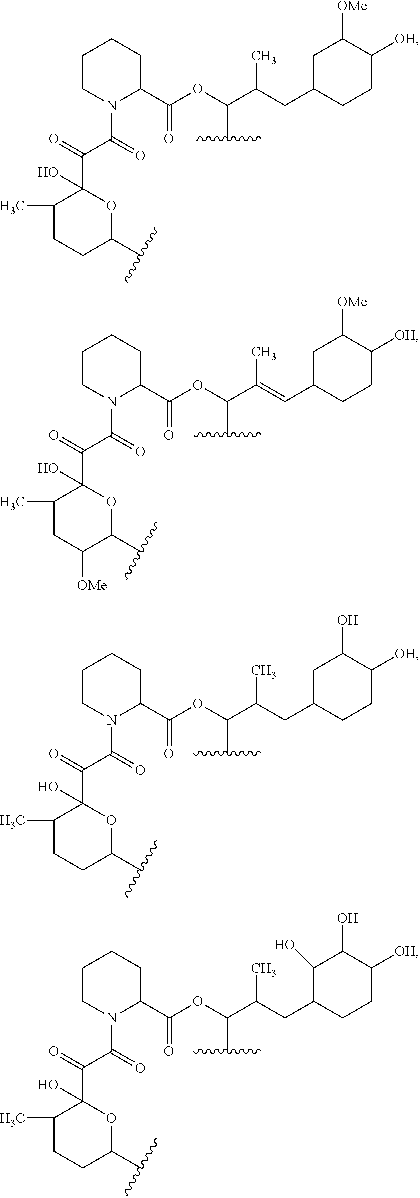 [SMILES: *C1CCC(C)C(O)(C(=O)C(=O)N2CCCCC2C(=O)OC(*)C(C)CC2CCC(O)C(OC)C2)O1 ; *C(OC(=O)C1CCCCN1C(=O)C(=O)C1(O)OC(*)C(OC)CC1C)/C(C)=C/C1CCC(O)C(OC)C1 ; *C1CCC(C)C(O)(C(=O)C(=O)N2CCCCC2C(=O)OC(*)C(C)CC2CCC(O)C(O)C2)O1 ; *C1CCC(C)C(O)(C(=O)C(=O)N2CCCCC2C(=O)OC(*)C(C)CC2CCC(O)C(O)C2O)O1]

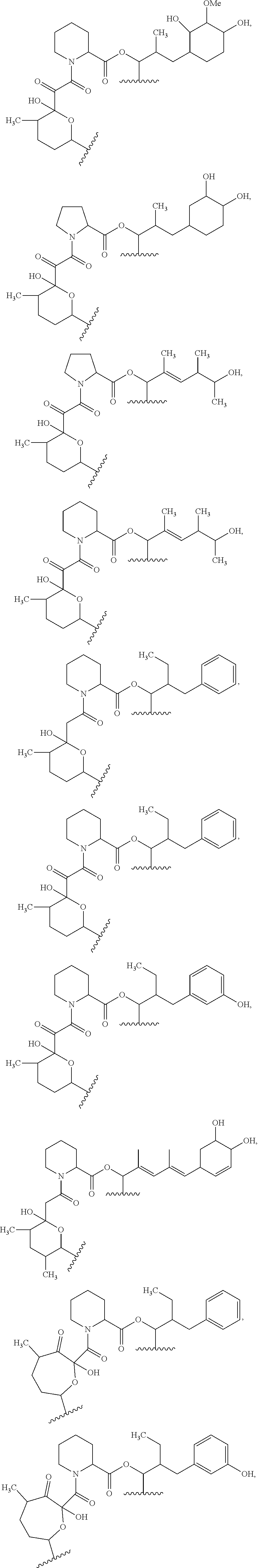

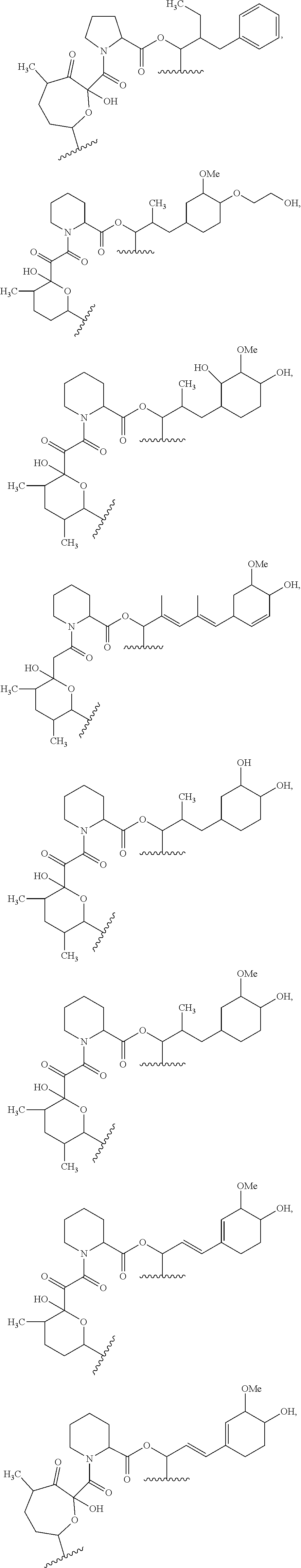

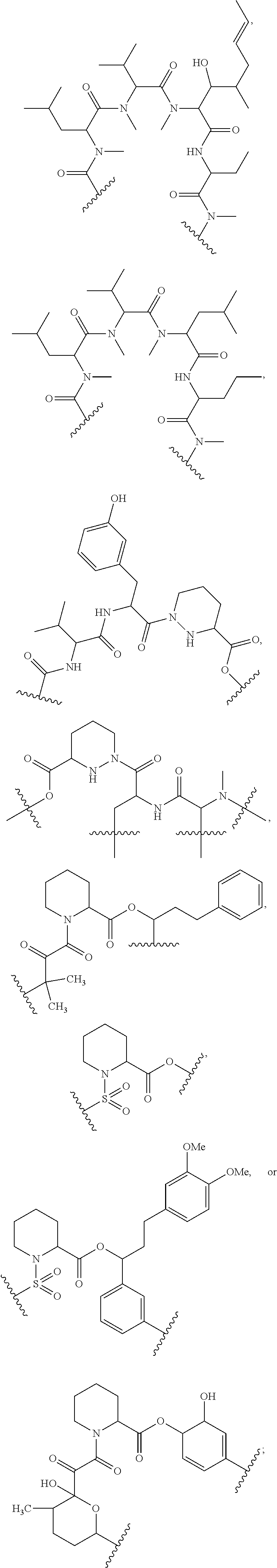

In certain embodiments, the presenter protein binding moiety has the structure of:

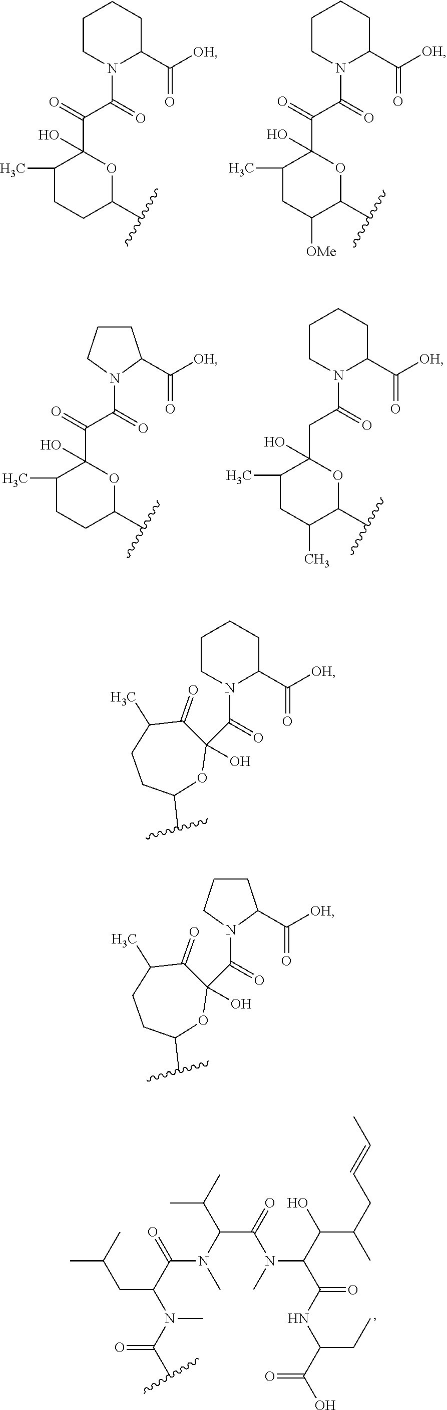

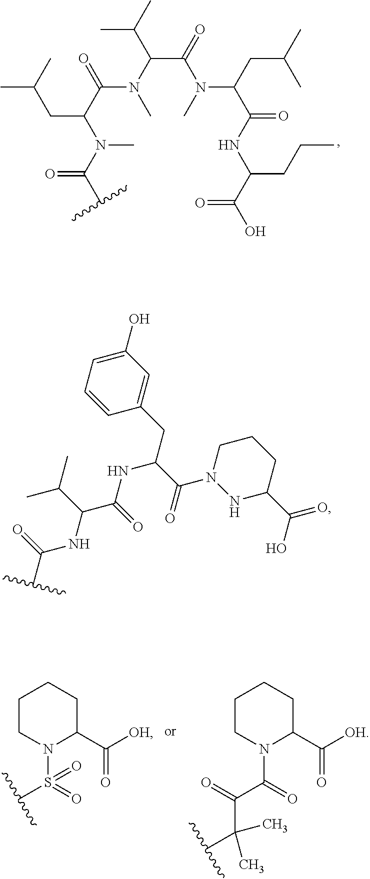 [SMILES: *C(=O)N(C)C(CC(C)C)C(=O)N(C)C(C(=O)N(C)C(CC(C)C)C(=O)NC(CCC)C(=O)O)C(C)C ; "," ; *C(=O)NC(C(=O)NC(Cc1cccc(O)c1)C(=O)N1CCCC(C(=O)O)N1)C(C)C ; *S(=O)(=O)N1CCCCC1C(=O)O ; "or" ; *C(C)(C)C(=O)C(=O)N1CCCCC1C(=O)O]

### Activity-Based Probes

Certain aziridine-containing probes are known in the art and may be used in conjunction with the methods of the present disclosure. These compounds may be useful in forming any of the complexes described herein. Exemplary aziridine-containing activity-based probes are shown below. Such probes may be modified using methods disclosed herein and known in the art to incorporate the aziridine moieties disclosed herein, such as replacing the original aziridine.

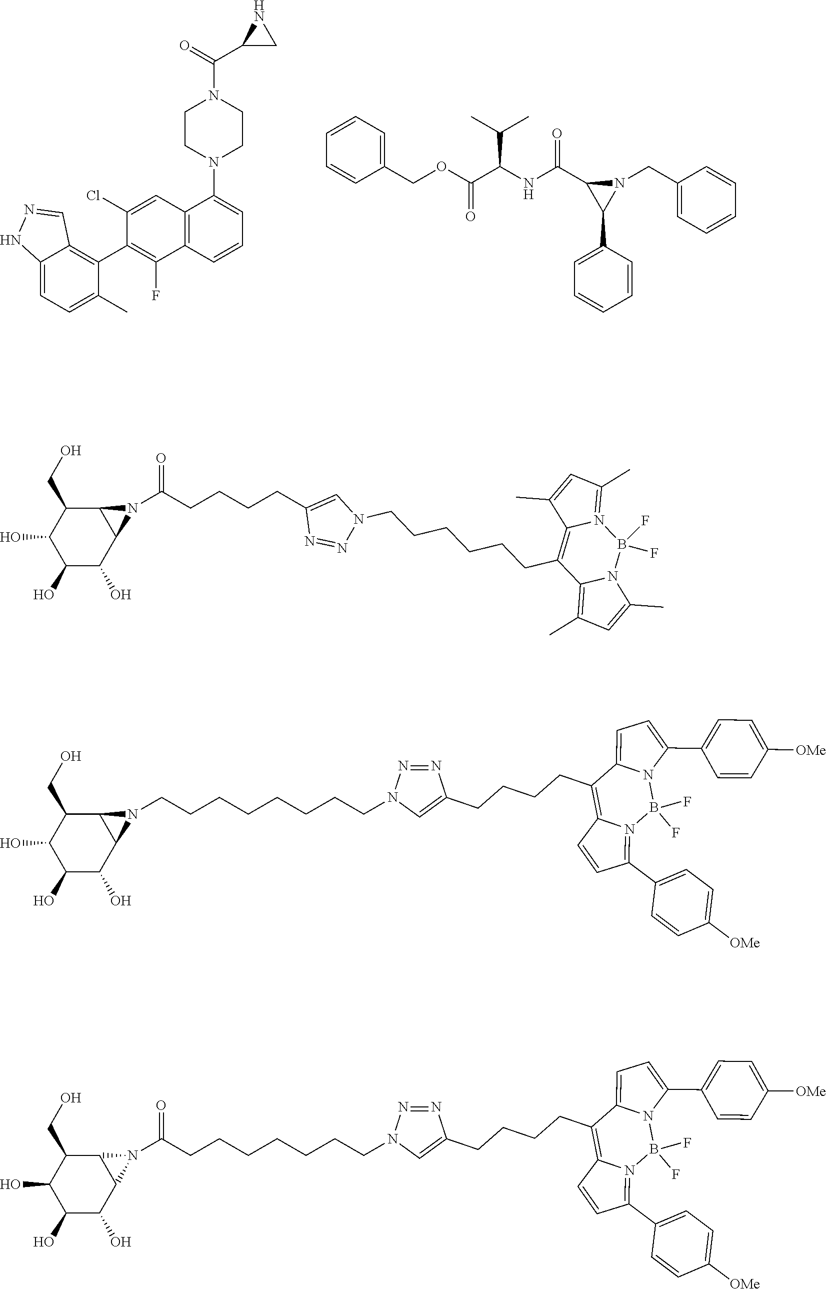

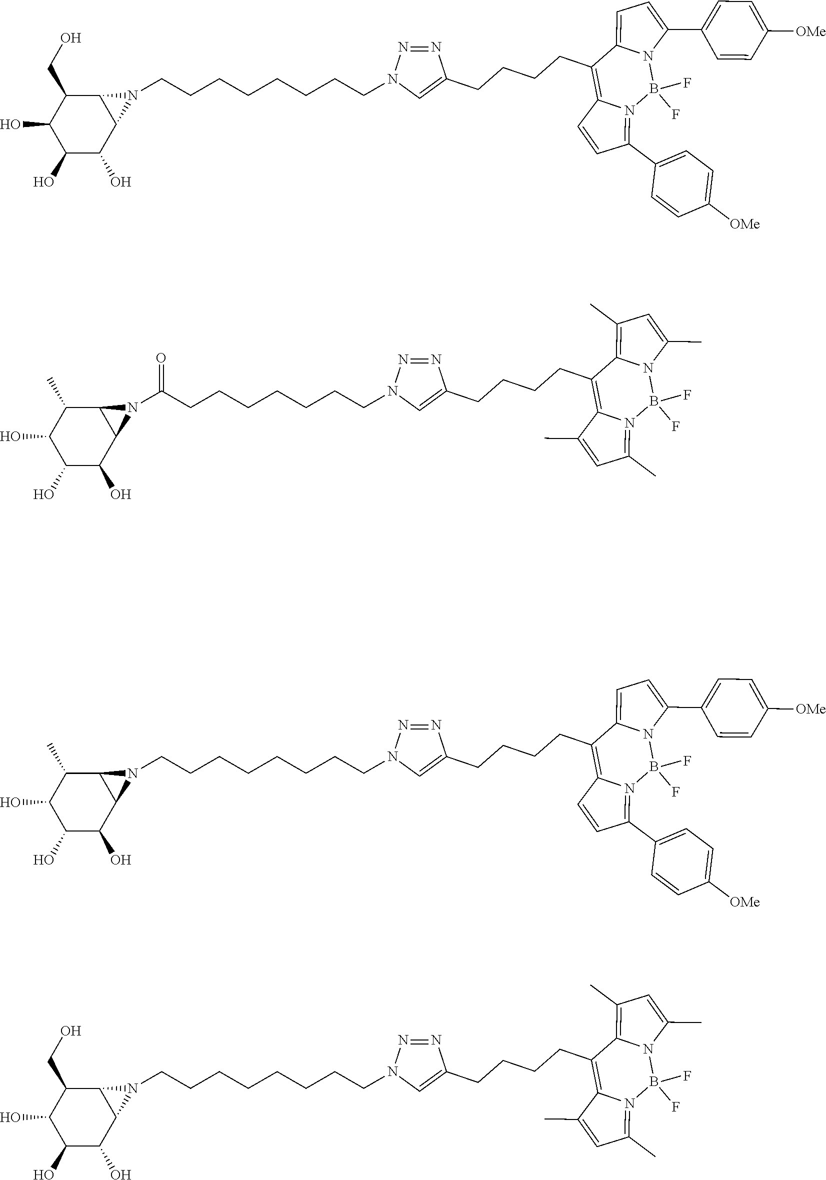

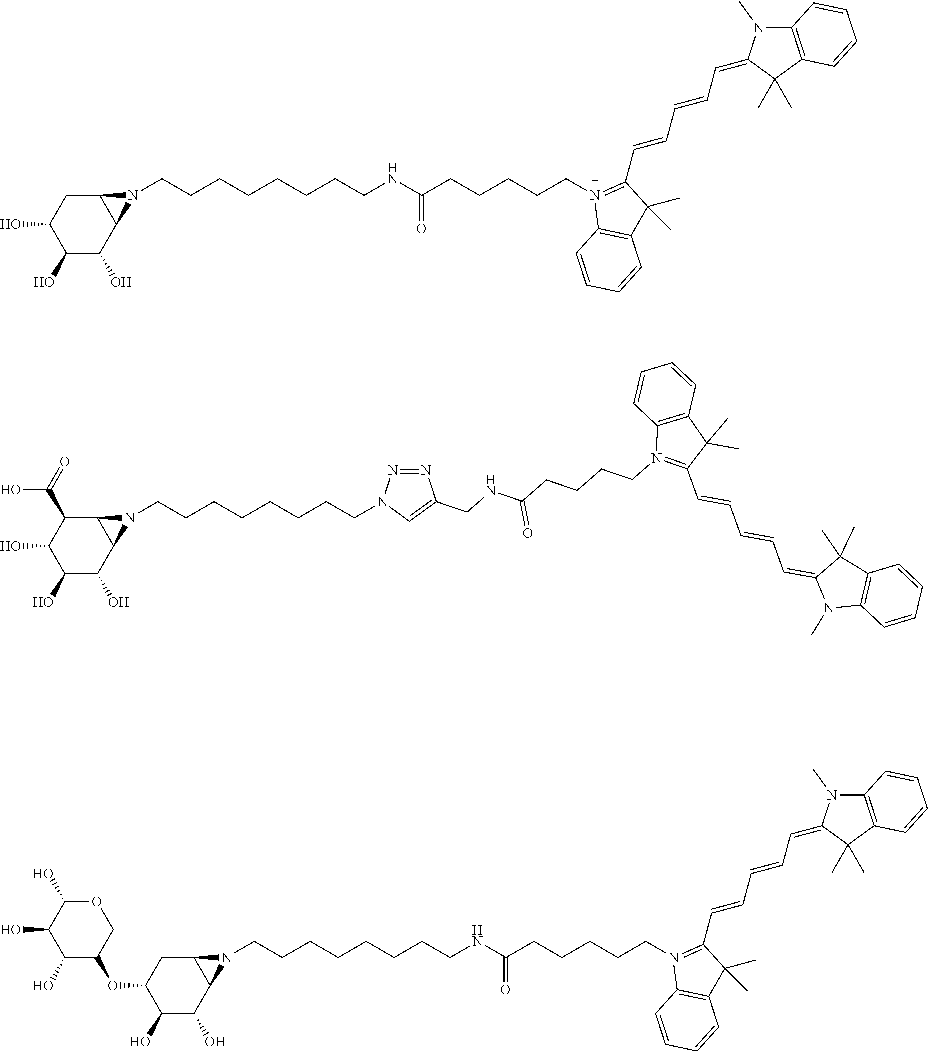

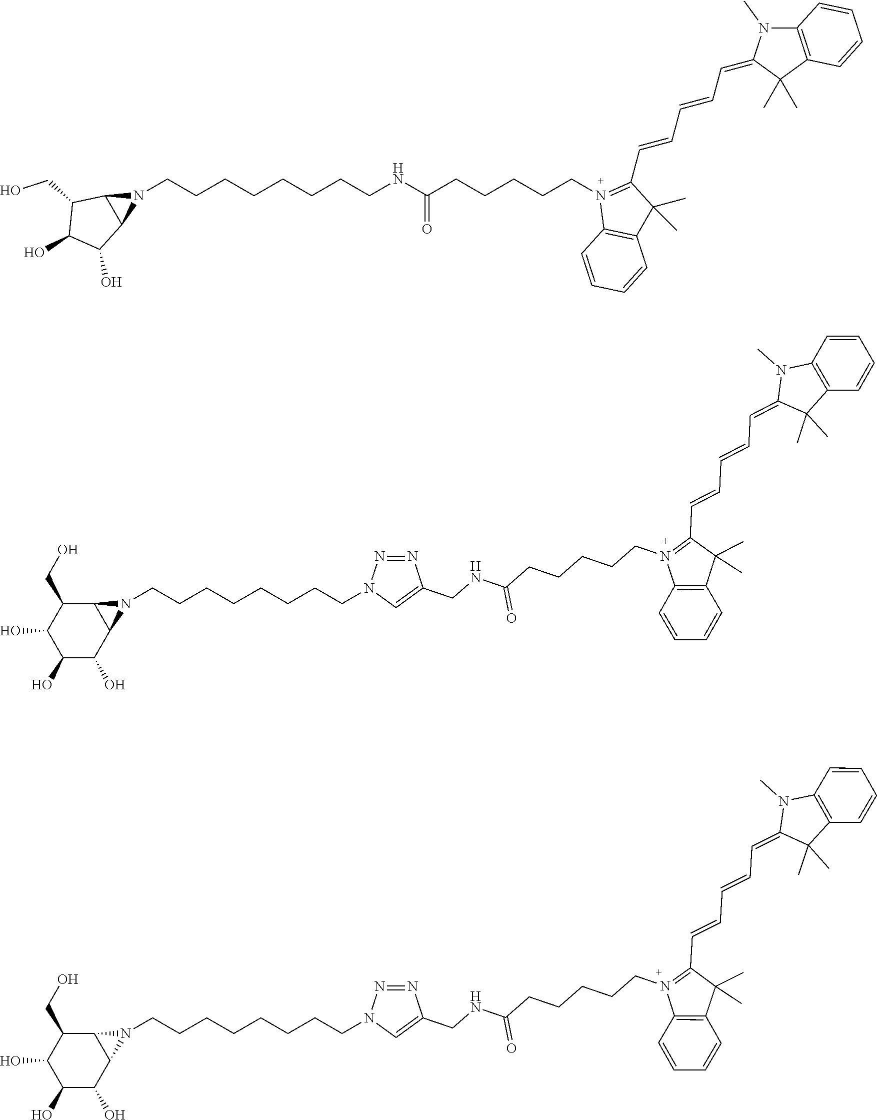

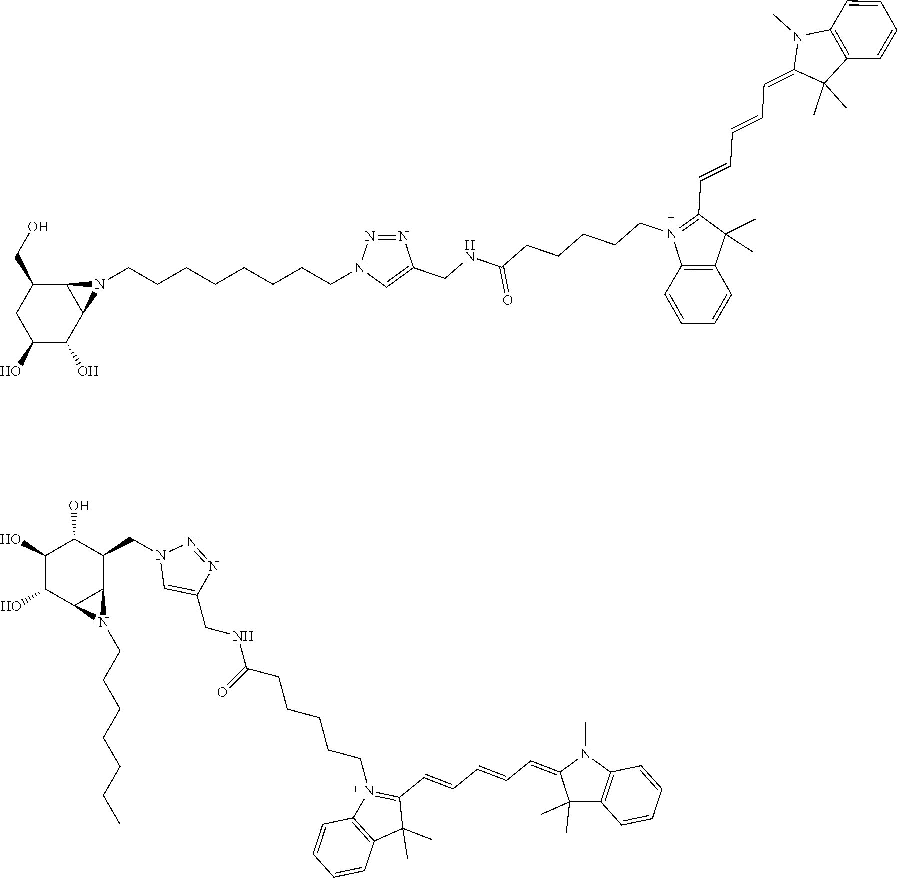

### Compound Characteristics

### Pharmacokinetic Parameters

Preliminary exposure characteristics of the compounds can be evaluated using, e.g., an in vivo Rat Early Pharmacokinetic (EPK) study design to show bioavailability. For example, Male Sprague-Dawley rats can be dosed via oral (PO) gavage in a particular formulation. Blood samples can then be collected from the animals at 6 timepoints out to 4 hours post-dose. Pharmacokinetic analysis can then be performed on the LC-MS/MS measured concentrations for each timepoint of each compound.

### Cell Permeability

In some embodiments, the compound is cell-penetrant. To determine permeability of a compound any method known in the art may be employed such as a Biosensor assay as described herein.

### Proteins

### Presenter Proteins

Presenter proteins can bind a small molecule to form a complex, which can bind to and modulate the activity of a target protein (e.g., a eukaryotic target protein such as a mammalian target protein or a fungal target protein or a prokaryotic target protein such as a bacterial target protein). In some embodiments, the presenter protein is a mammalian presenter protein (e.g., a human presenter protein). In some embodiments, the presenter protein is a fungal presenter protein. In certain embodiments, the presenter protein is a bacterial presenter protein. In some embodiments, the presenter protein is a plant presenter protein. In some embodiments, the presenter protein is a relatively abundant protein (e.g., the presenter protein is sufficiently abundant that participation in a tri-complex does not materially negatively impact the biological role of the presenter protein in a cell and/or viability or other attributes of the cell). In some embodiments, the presenter protein is more abundant than the target protein. In certain embodiments, the presenter protein is a protein that has chaperone activity within a cell. In some embodiments, the presenter protein has multiple natural interaction partners within a cell. In certain embodiments, the presenter protein is one which is known to bind a small molecule to form a binary complex that is known to or suspected of binding to and modulating the biological activity of a target protein. Immunophilins are a class of presenter proteins which are known to have these functions and include FKBPs and cyclophilins.

In some embodiments, a reference presenter protein exhibits peptidyl prolyl isomerase activity; in some embodiments, a presenter protein shows comparable activity to the reference presenter protein. In certain embodiments, the presenter protein is a member of the FKBP family (e.g., FKBP12, FKBP12.6, FKBP13, FKBP19, FKBP22, FKBP23, FKBP25, FKBP36, FKBP38, FKBP51, FKBP52, FKBP60, FKBP65, and FKBP133), a member of the cyclophilin family (e.g., PP1A, CYPB, CYPC, CYP40, CYPE, CYPD, NKTR, SRCyp, CYPH, CWC27, CYPL1, CYP60, CYPJ, PPIL4, PPIL6, RANBP2, PPWD1, PPIAL4A, PPIAL4B, PPIAL4C, PPIAL4D, or PPIAL4G), or PIN1. The “FKBP family” is a family of proteins that have prolyl isomerase activity and function as protein folding chaperones for proteins containing proline residues. Genes that encode proteins in this family include AIP, AIPL1, FKBP1A, FKBP1B, FKBP2, FKBP3, FKBP4, FKBP5, FKBP6, FKBP7, FKBP8, FKBP9, FKBP9L, FKBP10, FKBP11, FKBP14, FKBP15, and LOC541473.

The “cyclophilin family” is a family of proteins that bind to cyclosporine. Genes that encode proteins in this family include PPIA, PPIB, PPIC, PPID, PPIE, PPIF, PPIG, PPIH, SDCCAG-10, PPIL1, PPIL2, PPIL3, PPIL4, P270, PPWD1, and COAS-2. Exemplary cyclophilins include PP1A, CYPB, CYPC, CYP40, CYPE, CYPD, NKTR, SRCyp, CYPH, CWC27, CYPL1, CYP60, CYPJ, PPIL4, PPIL6, RANBP2, PPWD1, PPIAL4A, PPIAL4B, PPIAL4C, PPIAL4D, and PPIAL4G.

In some embodiments, a presenter protein is a chaperone protein such as GRP78/BiP, GRP94, GRP170, calnexin, calreticulin, HSP47, ERp29, Protein disulfide isomerase (PDI), and ERp57.

In some embodiments, a presenter protein is an allelic variant or splice variant of a FKBP or cyclophilin disclosed herein.

In some embodiments, a presenter protein is a polypeptide whose amino acid sequence i) shows significant identity with that of a reference presenter protein; ii) includes a portion that shows significant identity with a corresponding portion of a reference presenter protein; and/or iii) includes at least one characteristic sequence found in presenter protein. In many embodiments, identity is considered “significant” for the purposes of defining a presenter protein if it is above 80%, 81%, 82%, 83%, 84%, 85%, 86%, 87%, 89%, 90%, 91%, 92%, 93%, 94%, 95%, 96%, 97%, 98%, 99%, or higher. In some embodiments, the portion showing significant identity has a length of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 300, 350, 450, 500, 550, 600 amino acids or more.

Representative presenter proteins are encoded by the genes or homologs thereof listed in Table 3; in some embodiments, a reference presenter protein is encoded by a gene set forth in Table 3. Also, those of ordinary skill in the art, referring to Table 3, can readily identify sequences that are characteristic of presenter proteins generally, and/or of particular subsets of presenter proteins.

TABLE 3
Genes that Encode Selected Presenter Proteins

| | Uniprot |
|---|---|
| | Accession |
| Gene Name | Number |
| AIP | O00170 |
| AIPL1 | Q9NZN9 |
| FKBP1A | P62942 |
| FKBP1B | P68106 |
| FKBP2 | P26885 |
| FKBP3 | Q00688 |
| FKBP4 | Q02790 |
| FKBP5 | Q13451 |
| FKBP6 | O75344 |
| FKBP7 | Q9Y680 |
| FKBP8 | Q14318 |
| FKBP9 | O95302 |
| FKBP9L | Q75LS8 |
| FKBP10 | Q96AY3 |
| FKBP11 | Q9NYL4 |
| FKBP14 | Q9NWM8 |
| FKBP15 | Q5T1M5 |
| LOC541473 | — |
| PPIA | Q567Q0 |
| PPIB | P23284 |
| PPIC | P45877 |
| PPID | Q08752 |
| PPIE | Q9UNP9 |
| PPIG | Q13427 |
| PPIH | O43447 |
| PPIL1 | Q9Y3C6 |
| PPIL2 | Q13356 |
| PPIL3 | Q9H2H8 |
| PPIL4 | Q8WUA2 |
| PPIL5 | Q32Q17 |
| PPIL6 | Q8IXY8 |
| PPWD1 | Q96BP3 |

### Target Proteins

A target protein (e.g., a eukaryotic target protein such as a mammalian target protein or a fungal target protein or a prokaryotic target protein such as a bacterial target protein) is a protein which mediates a disease condition or a symptom of a disease condition. As such, a desirable therapeutic effect can be achieved by modulating (inhibiting or increasing) its activity. Target proteins useful in the complexes and methods of the invention include those which do not naturally associate with a presenter protein, e.g., those which have an affinity for a presenter protein in the absence of a binary complex with a compound of the invention of greater than 1 μM, preferably greater than 5 μM, and more preferably greater than 10 μM. Alternatively, target proteins which do not naturally associate with a presenter protein are those which have an affinity for a compound of the invention in the absence of a binary complex greater than 1 μM, preferably greater than 5 μM, and more preferably greater than 10 μM. In another alternative, target proteins which do not naturally associate with a presenter protein are those which have an affinity for a binary complex of cyclosporine, rapamycin, or FK506 and a presenter protein (e.g., FKBP) of greater than 1 μM, preferably greater than 5 μM, and more preferably greater than 10 μM. In yet another alternative, target proteins which do not naturally associate with a presenter protein are those which are other than calcineurin or mTOR. The selection of suitable target proteins for the complexes and methods of the invention may depend on the presenter protein. For example, target proteins that have low affinity for a cyclophilin may have high affinity for an FKBP and would not be used together with the latter.

Target proteins can be naturally occurring, e.g., wild type. Alternatively, a target protein can vary from the wild type protein but still retain biological function, e.g., as an allelic variant, a splice mutant or a biologically active fragment.

In some embodiments, a target protein is a transmembrane protein. In some embodiments, a target protein has a coiled coil structure. In certain embodiments, a target protein is one protein of a dimeric complex.

In some embodiments, a target protein of the invention includes one or more surface sites (e.g., a flat surface site) characterized in that, in the absence of forming a presenter protein/compound complex, small molecules typically demonstrate low or undetectable binding to the site(s). In some embodiments, a target protein includes one or more surface sites (e.g., a flat surface site) to which, in the absence of forming a presenter protein/compound complex, a particular small molecule (e.g., the compound) shows low or undetectable binding (e.g., binding at least 2, 3, 4, 5, 6, 7, 8, 9, 10, 20, 30, 40, 50, 100 fold or more lower than that observed with a presenter protein/compound complex involving the same compound). In some embodiments, a target protein has a surface characterized by one or more sites (and, in some embodiments, an entire surface) that lack(s) any a traditional binding pocket, for example, a cavity or pocket on the protein structure with physiochemical and/or geometric properties comparable to proteins whose activity has been modulated by one or more small molecules. In certain embodiments, a target protein has a traditional binding pocket and a site for a protein-protein interaction. In some embodiments, a target protein is an undruggable target, for example, a target protein is not a member of a protein family which is known to be targeted by drugs and/or does not possess a binding site that is expected (e.g., according to art-accepted understanding, as discussed herein) to be suitable for binding to a small molecule.

In some embodiments, the target protein is a GTPase such as DIRAS1, DIRAS2, DIRAS3, ERAS, GEM, HRAS, KRAS, MRAS, NKIRAS1, NKIRAS2, NRAS, RALA, RALB, RAP1A, RAP1B, RAP2A, RAP2B, RAP2C, RASD1, RASD2, RASL10A, RASL10B, RASL11A, RASL11B, RASL12, REM1, REM2, RERG, RERGL, RRAD, RRAS, RRAS2, RHOA, RHOB, RHOBTB1, RHOBTB2, RHOBTB3, RHOC, RHOD, RHOF, RHOG, RHOH, RHOJ, RHOQ, RHOU, RHOV, RND1, RND2, RND3, RAC1, RAC2, RAC3, CDC42, RAB1A, RAB1B, RAB2, RAB3A, RAB3B, RAB3C, RAB3D, RAB4A, RAB4B, RABSA, RAB5B, RAB5C, RAB6A, RAB6B, RAB6C, RAB7A, RAB7B, RAB7L1, RAB8A, RAB8B, RAB9, RAB9B, RABL2A, RABL2B, RABL4, RAB10, RAB11A, RAB11B, RAB12, RAB13, RAB14, RAB15, RAB17, RAB18, RAB19, RAB20, RAB21, RAB22A, RAB23, RAB24, RAB25, RAB26, RAB27A, RAB27B, RAB28, RAB2B, RAB30, RAB31, RAB32, RAB33A, RAB33B, RAB34, RAB35, RAB36, RAB37, RAB38, RAB39, RAB39B, RAB40A, RAB40AL, RAB40B, RAB40C, RAB41, RAB42, RAB43, RAP1A, RAP1B, RAP2A, RAP2B, RAP2C, ARF1, ARF3, ARF4, ARF5, ARF6, ARL1, ARL2, ARL3, ARL4, ARL5, ARL5C, ARL6, ARL7, ARL8, ARL9, ARL10A, ARL10B, ARL10C, ARL11, ARL13A, ARL13B, ARL14, ARL15, ARL16, ARL17, TRIM23, ARL4D, ARFRP1, ARL13B, RAN, RHEB, RHEBL1, RRAD, GEM, REM, REM2, RIT1, RIT2, RHOT1, or RHOT2. In some embodiments, the target protein is a GTPas activating protein such as NF1, IQGAP1, PLEXIN-B1, RASAL1, RASAL2, ARHGAP5, ARHGAP8, ARHGAP12, ARHGAP22, ARHGAP25, BCR, DLC1, DLC2, DLC3, GRAF, RALBP1, RAP1GAP, SIPA1, TSC2, AGAP2, ASAP1, or ASAP3. In some embodiments, the target protein is a Guanine nucleotide-exchange factor such as CNRASGEF, RASGEF1A, RASGRF2, RASGRP1, RASGRP4, SOS1, RALGDS, RGL1, RGL2, RGR, ARHGEF10, ASEF/ARHGEF4, ASEF2, DBS, ECT2, GEF-H₁, LARG, NET1, OBSCURIN, P-REX1, P-REX2, PDZ-RHOGEF, TEM4, TIAM1, TRIO, VAV1, VAV2, VAV3, DOCK1, DOCK2, DOCK3, DOCK4, DOCK8, DOCK10, C₃G, BIG2/ARFGEF2, EFA6, FBX8, or GEP100. In certain embodiments, the target protein is a protein with a protein-protein interaction domain such as ARM; BAR; BEACH; BH; BIR; BRCT; BROMO; BTB; C₁; C₂; CARD; CC; CALM; CH; CHROMO; CUE; DEATH; DED; DEP; DH; EF-hand; EH; ENTH; EVH1; F-box; FERM; FF; FH2; FHA; FYVE; GAT; GEL; GLUE; GRAM; GRIP; GYF; HEAT; HECT; IQ; LRR; MBT; MH1; MH2; MIU; NZF; PAS; PB1; PDZ; PH; POLO-Box; PTB; PUF; PWWP; PX; RGS; RING; SAM; SC; SH2; SH3; SOCS; SPRY; START; SWIRM; TIR; TPR; TRAF; SNARE; TUBBY; TUDOR; UBA; UEV; UIM; VHL; VHS; WD40; WW; SH2; SH3; TRAF; Bromodomain; or TPR. In some embodiments, the target protein is a heat shock protein such as Hsp20, Hsp27, Hsp70, Hsp84, alpha B crystalline, TRAP-1, hsf1, or Hsp90. In certain embodiments, the target protein is an ion channel such as Cav2.2, Cav3.2, IKACh, Kv1.5, TRPA1, NAv1.7, Nav1.8, Nav1.9, P2X3, or P2X4. In some embodiments, the target protein is a coiled-coil protein such as geminin, SPAG4, VAV1, MAD1, ROCK1, RNF31, NEDP1, HCCM, EEA1, Vimentin, ATF4, Nemo, SNAP25, Syntaxin 1a, FYCO1, or CEP250. In certain embodiments, the target protein is a kinase such as ABL, ALK, AXL, BTK, EGFR, FMS, FAK, FGFR1, 2, 3, 4, FLT3, HER2/ErbB2, HER3/ErbB3, HER4/ErbB4, IGF1R, INSR, JAK1, JAK2, JAK3, KIT, MET, PDGFRA, PDGFRB, RET RON, ROR1, ROR2, ROS, SRC, SYK, TIE1, TIE2, TRKA, TRKB, KDR, AKT1, AKT2, AKT3, PDK1, PKC, RHO, ROCK1, RSK1, RKS2, RKS3, ATM, ATR, CDK1, CDK2, CDK3, CDK4, CDK5, CDK6, CDK7, CDK8, CDK9, CDK10, ERK1, ERK2, ERK3, ERK4, GSK3A, GSK3B, JNK1, JNK2, JNK3, AurA, ARuB, PLK1, PLK2, PLK3, PLK4, IKK, KIN1, cRaf, PKN3, c-Src, Fak, PyK2, or AMPK. In some embodiments, the target protein is a phosphatase such as WIP1, SHP2, SHP1, PRL-3, PTP1B, or STEP. In certain embodiments the target protein is a ubiquitin ligase such as BMI-1, MDM2, NEDD4-1, Beta-TRCP, SKP2, E6AP, or APC/C. In some embodiments, the target protein is a chromatin modifier/remodeler such as a chromatin modifier/remodeler encoded by the gene BRG1, BRM, ATRX, PRDM3, ASH1L, CBP, KAT6A, KAT6B, MLL, NSD1, SETD2, EP300, KAT2A, or CREBBP. In some embodiments, the target protein is a transcription factor such as a transcription factor encoded by the gene EHF, ELF1, ELF3, ELF4, ELF5, ELK1, ELK3, ELK4, ERF, ERG, ETS1, ETV1, ETV2, ETV3, ETV4, ETV5, ETV6, FEV, FLI1, GAVPA, SPDEF, SP11, SPIC, SPIB, E2F1, E2F2, E2F3, E2F4, E2F7, E2F8, ARNTL, BHLHA15, BHLHB2, BHLBHB3, BHLHE22, BHLHE23, BHLHE41, CLOCK, FIGLA, HAS5, HES7, HEY1, HEY2, ID4, MAX, MESP1, MLX, MLXIPL, MNT, MSC, MYF6, NEUROD2, NEUROG2, NHLH1, OLIG1, OLIG2, OLIG3, SREBF2, TCF3, TCF4, TFAP4, TFE3, TFEB, TFEC, USF1, ARF4, ATF7, BATF3, CEBPB, CEBPD, CEBPG, CREB3, CREB3L1, DBP, HLF, JDP2, MAFF, MAFG, MAFK, NRL, NFE2, NFIL3, TEF, XBP1, PROX1, TEAD1, TEAD3, TEAD4, ONECUT3, ALX3, ALX4, ARX, BARHL2, BARX, BSX, CART1, CDX1, CDX2, DLX1, DLX2, DLX3, DLX4, DLX5, DLX6, DMBX1, DPRX, DRGX, DUXA, EMX1, EMX2, EN1, EN2, ESX1, EVX1, EVX2, GBX1, GBX2, GSC, GSC2, GSX1, GSX2, HESX1, HMX1, HMX2, HMX3, HNF1A, HNF1B, HOMEZ, HOXA1, HOXA10, HOXA13, HOXA2, HOXAB13, HOXB2, HOXB3, HOXB5, HOXC10, HOXC11, HOXC12, HOXC13, HOXD11, HOXD12, HOXD13, HOXD8, IRX2, IRX5, ISL2, ISX, LBX2, LHX2, LHX6, LHX9, LMX1A, LMX1B, MEIS1, MEIS2, MEIS3, MEOX1, MEOX2, MIXL1, MNX1, MSX1, MSX2, NKX2-3, NKX2-8, NKX3-1, NKX3-2, NKX6-1, NKX6-2, NOTO, ONECUT1, ONECUT2, OTX1, OTX2, PDX1, PHOX2A, PHOX2B, PITX1, PITX3, PKNOX1, PROP1, PRRX1, PRRX2, RAX, RAXL1, RHOXF1, SHOX, SHOX2, TGIF1, TGIF2, TGIF2LX, UNCX, VAX1, VAX2, VENTX, VSX1, VSX2, CUX1, CUX2, POU F1, POU2F1, POU2F2, POU2F3, POU3F1, POU3F2, POU3F3, POU3F4, POU4F1, POU4F2, POU4F3, POU5F1P1, POU6F2, RFX2, RFX3, RFX4, RFX5, TFAP2A, TFAP2B, TFAP2C, GRHL1, TFCP2, NFIA, NFIB, NFIX, GCM1, GCM2, HSF1, HSF2, HSF4, HSFY2, EBF1, IRF3, IRF4, IRF5, IRF7, IRF8, IRF9, MEF2A, MEF2B, MEF2D, SRF, NRF1, CPEB1, GMEB2, MYBL1, MYBL2, SMAD3, CENPB, PAX1, PAX2, PAX9, PAX3, PAX4, PAX5, PAX6, PAX7, BCL6B, EGR1, EGR2, EGR3, EGR4, GLIS1, GLIS2, GLI2, GLIS3, HIC2, HINFP1, KLF13, KLF14, KLF16, MTF1, PRDM1, PRDM4, SCRT1, SCRT2, SNAI2, SP1, SP3, SP4, SP8, YY1, YY2, ZBED1, ZBTB7A, ZBTB7B, ZBTB7C, ZIC1, ZIC3, ZIC4, ZNF143, ZNF232, ZNF238, ZNF282, ZNF306, ZNF410, ZNF435, ZBTB49, ZNF524, ZNF713, ZNF740, ZNF75A, ZNF784, ZSCAN4, CTCF, LEF1, SOX10, SOX14, SOX15, SOX18, SOX2, SOX21, SOX4, SOX7, SOX8, SOX9, SRY, TCF7L1, FOXO3, FOXB1, FOXC1, FOXC2, FOXD2, FOXD3, FOXG1, FOXI1, FOXJ2, FOXJ3, FOXK1, FOXL1, FOXO1, FOXO4, FOXO6, FOXP3, EOMES, MGA, NFAT5, NFATC1, NFKB1, NFKB2, TP63, RUNX2, RUNX3, T, TBR1, TBX1, TBX15, TBX19, TBX2, TBX20, TBX21, TBX4, TBX5, AR, ESR1, ESRRA, ESRRB, ESRRG, HNF4A, NR2C₂, NR2E1, NR2F1, NR2F6, NR3C₁, NR3C₂, NR4A2, RARA, RARB, RARG, RORA, RXRA, RXRB, RXRG, THRA, THRB, VDR, GATA3, GATA4, or GATA5; or C-myc, Max, Stat3, androgen receptor, C-Jun, C-Fox, N-Myc, L-Myc, MITF, Hif-1alpha, Hif-2alpha, Bcl6, E2F1, NF-kappaB, Stat5, or ER(coact). In certain embodiments, the target protein is TrkA, P2Y14, mPEGS, ASK1, ALK, Bcl-2, BCL-XL, mSIN1, RORγt, IL17RA, eIF4E, TLR7R, PCSK9, IgE R, CD40, CD40L, Shn-3, TNFR1, TNFR2, IL31RA, OSMR, IL12beta1,2, Tau, FASN, KCTD 6, KCTD 9, Raptor, Rictor, RALGAPA, RALGAPB, Annexin family members, BCOR, NCOR, beta catenin, AAC 11, PLD1, PLD2, Frizzled7, RaLP, MLL-1, Myb, Ezh2, RhoGD12, EGFR, CTLA4R, GCGC (coact), Adiponectin R2, GPR 81, IMPDH2, IL-4R, IL-13R, IL-1R, IL2-R, IL-6R, IL-22R, TNF-R, TLR4, Nrlp3, or OTR.

### Methods of Synthesis

Methods of synthesizing cyclopropyl aziridines that are disclosed herein are known in the art. The methods may include those shown in the following scheme.

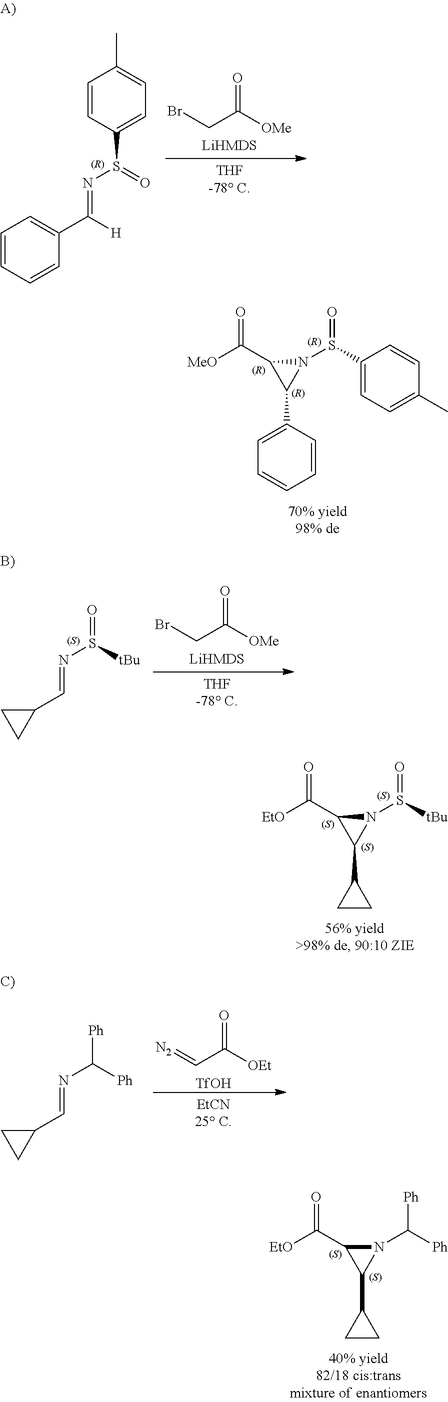

(2R,3R)-beta-phenyl aziridines have been synthesized using the Davis auxiliary (para-toluenesulfinamide) (Davis et al., J. Org. Chem. 1994, 59 (12), 3243-3245; Davis et al., J. Org. Chem. 1999, 64 (20), 7559-7567). See letter A, above.

(2S,3S)-beta-cyclopropyl aziridines (letter B, above) can be synthesized using the Ellman's auxiliary (tert-butylsulfinamide) (Sols et al., Org. Biomol. Chem. 2011, 9 (14), 5034).

Beta-cyclopropyl aziridines can also be prepared by reaction of the diphenylmethyl imine with the diazo ester under acidic conditions (Williams, et al., J. Am. Chem. Soc. 2004, 126 (6), 1612-1613). However, this method is not asymmetric and results in a mixture of cis- and trans-isomers and a mixture of enantiomers.

Alternatively, beta-cyclopropyl aziridines can be made on a large scale using the following scheme:

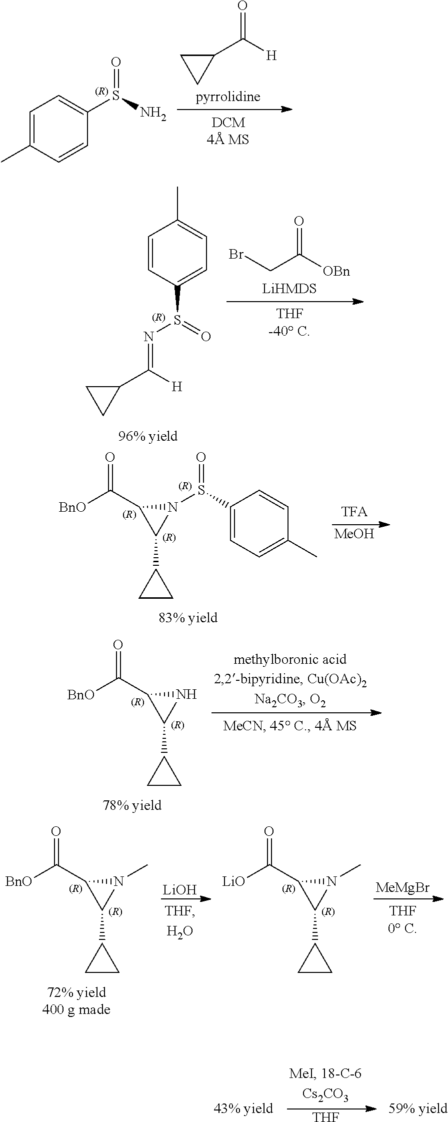

The (2R,3R)-beta-cyclopropyl aziridine can be synthesized by the reaction of the (R)-para-toluenesulfinamide with benzyl 2-bromoacetate and LiHMDS. Removal of the chiral auxiliary is achieved using either TFA or methyl Grignard. Methylation of the aziridine nitrogen is performed by either Chan-Lam coupling with methyl boronic acid or alkylation with methyl iodide.

### Complexes

### Presenter Protein/Compound Complexes

In naturally occurring protein-protein interactions, the binding event is driven largely by hydrophobic residues on flat surface sites of the two proteins, in contrast to many small molecule-protein interactions which are driven by interactions between the small molecule in a cavity or pocket on the protein. The hydrophobic residues on the flat surface site form hydrophobic hot spots on the two interacting proteins wherein most of the binding interactions between the two proteins are van der Waals interactions. Small molecules may be used as portable hotspots for proteins which are lacking one (e.g., presenter proteins) through the formation of complexes (e.g., a presenter protein/compound complex) to participate in pseudo protein-protein interactions (e.g., forming a tri-complex with a target protein).

Many mammalian proteins are able to bind to any of a plurality of different partners; in some cases, such alternative binding interactions contribute to biological activity of the proteins. Many of these proteins adapt the inherent variability of the hot spot protein regions to present the same residues in different structural contexts. More specifically, the protein-protein interactions can be mediated by a class of natural products produced by a select group of fungal and bacterial species. These molecules exhibit both a common structural organization and resultant functionality that provides the ability to modulate protein-protein interaction. These molecules contain a presenter protein binding moiety that is highly conserved and a target protein interacting moiety that exhibits a high degree of variability among the different natural products. The presenter protein binding moiety confers specificity for the presenter protein and allows the molecule to bind to the presenter protein to form a binary complex; the mammalian target protein interacting moiety confers specificity for the target protein and allows the binary complex to bind to the target protein, typically modulating (e.g., positively or negatively modulating) its activity.

These natural products are presented by presenter proteins, such as FKBPs and cyclophilins and act as diffusible, cell-penetrant, orally bio-available adaptors for protein-protein interactions. Examples include well known and clinically relevant molecules such as Rapamycin (Sirolimus), FK506 (Tacrolimus), and Cyclosporin. In brief, these molecules bind endogenous intracellular presenter proteins, the FKBPs e.g., rapamycin and FK506 or cyclophilins e.g., diluents, and the resulting binary complexes of presenter protein-bound molecules selectively bind and inhibit the activity of intracellular target proteins. Formation of a tri-complex between the presenter protein, the molecule, and the target protein is driven by both protein-molecule and protein-protein interactions and both are required for inhibition of the target protein. In the example of the FKBP-rapamycin complex, the intracellular target is the serine-threonine kinase mTOR, whereas for FKBP-FK506 complex, the intracellular target is the phosphatase calcineurin. Of particular interest in the preceding two examples, FKBP12 is utilized as a partner presentation protein by both the rapamycin and FK506 presentation ligands. Moreover, the sub-structure components of rapamycin and FK506 responsible for binding to FKBP12 are closely related structurally, i.e., the so-called “Conserved Region,” but it is the dramatic structural differences between rapamycin and FK506 in the non FKBP12-binding regions, i.e., the “Variable Region,” that results in the specific targeting of two distinct intracellular proteins, mTOR and calcineurin, respectively. In this fashion, the Variable Regions of rapamycin and FK506 are serving as contributors to the binding energy necessary for enabling presenter protein-target protein interaction.

In some embodiments, a presenter protein/compound complexes of the invention bind to a target protein with at least 5-fold (e.g., at least 10-fold, at least 20-fold, at least 30-fold, at least 40-fold, at least 50-fold, at least 100-fold) greater affinity than the complex binds to each of mTOR and/or calcineurin.

In some embodiments, a presenter protein/compound complexes of the invention bind to a target protein with at least 5-fold (e.g., at least 10-fold, at least 20-fold, at least 30-fold, at least 40-fold, at least 50-fold, at least 100-fold) greater affinity than the affinity of the compound to a target protein when the compound is not bound in a complex with a presenter protein.

In certain embodiments, a presenter protein/compound complexes of the invention bind to a target protein with at least 5-fold (e.g., at least 10-fold, at least 20-fold, at least 30-fold, at least 40-fold, at least 50-fold, at least 100-fold) greater affinity than the affinity of the presenter protein to a target protein when the presenter protein is not bound in a complex with a compound.

In some embodiments, a presenter protein/compound complexes of the invention inhibit a naturally occurring interaction between a target protein and a ligand, such as a protein or a small molecule that specifically binds to the target protein.

In certain embodiments, when the presenter protein is a prolyl isomerase, the prolyl isomerase activity is inhibited by formation of the presenter protein/compound complex. In some embodiments of the presenter protein/compound complexes of the invention, the compound specifically binds to said presenter protein with a K_{D} of less than 10 μM (e.g., less than 5 μM, less than 1 μM, less than 500 nM, less than 200 nM, less than 100 nM, less than 75 nM, less than 50 nM, less than 25 nM, less than 10 nM) or inhibits the peptidyl-prolyl isomerase activity of the presenter protein, for example, with an IC₅₀ of less than 1 μM (e.g., less than 0.5 μM, less than 0.1 μM, less than 0.05 μM, less than 0.01 μM).

### Compound/Target Protein Complexes

The compounds of the present disclosure may be useful for forming complexes with a target protein. The complexes can be formed by way of non-covalent interactions (e.g., van der Waals interactions or pi-interactions). Alternatively, or in addition, such complexes may be formed by way of “cross-linking” the target by formation of a covalent bond between the compound and the target protein. The compounds described herein may contain an electrophilic aziridine group capable of reaction with a nucleophilic residue of the target protein (e.g., an aspartic acid, glutamic acid, cysteine, glutamine, asparagine, lysine, or histidine residue).

The inclusion of a carbocation stabilizing electron donating group at a position beta to a carbonyl substituent may aid in promoting cross-linking with a target protein. For example, cyclopropyl substituents aid in allowing the aziridine to react with a nucleophilic residue (e.g., an aspartic acid residue). Without being bound by theory, the inventors postulate that in situ protonation of the weakly basic aziridine nitrogen enhances reactivity of the aziridine ring. Stabilization of a nascent carbocation (δ⁺) p-orbital at the aziridine beta-carbon by the pseudo aromatic cyclopropyl (σ_{c-c} bonds) moiety further facilitates a reaction with a low reactivity and highly solvated aspartate anion.

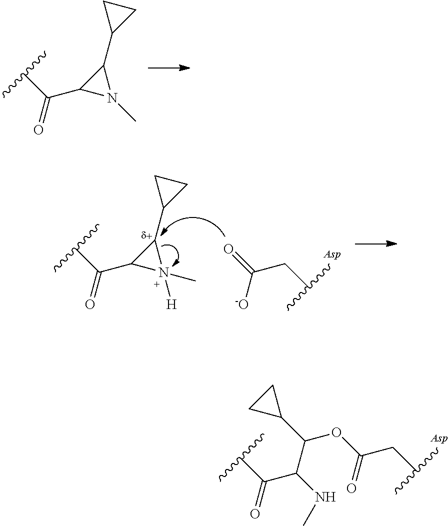

Beyond a beta-cyclopropyl group, additional moieties may be beneficial at stabilizing carbocations, either through hyperconjugation or resonance, including, but not limited to aryl, vinyl, ynyl, and cubyl groups.

In some embodiments, a compound/target protein complex of the disclosure has the structure of Formula III:

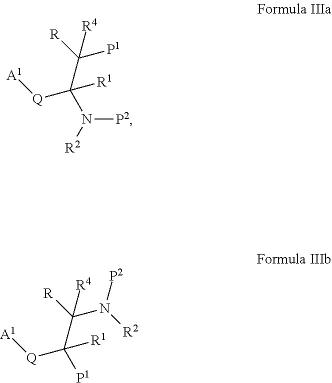

wherein A¹ is a monovalent organic moiety;
Q is

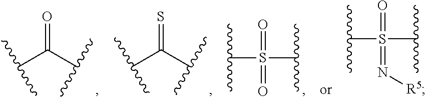

P¹ is A², and P² is hydrogen; or P¹ is hydroxyl, and P² is A²;
A² is the target protein;
R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl or —Si(R^{1a})₃;
or R and R¹ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R and R² combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and R¹ and R⁴ are each, independently, hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a}); or
R and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R² combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and each of R and R⁴ is, independently, hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R² and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and each of R¹ and R is, independently, hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
each R^{1a}, R^{2a}, R^{2c}, R^{2d}, and R^{2e} is, independently, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl;
R^{2b} is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted 5- to 10-membered heterocycloalkenyl, optionally substituted 8- to 10-membered heterocycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁵ is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl,
and wherein each hydrogen is independently, optionally, isotopically enriched for deuterium.

In some embodiments, at least one of R and R⁴ is not hydrogen.

In some embodiments, the compound/target protein complex has the structure of Formula IIIa-1:

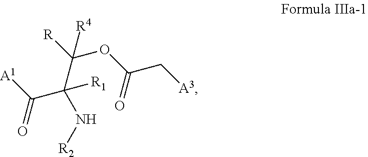

wherein A³ is the rest of the target protein.

In some embodiments, the compound/target protein complex has the structure of Formula IIIa-2:

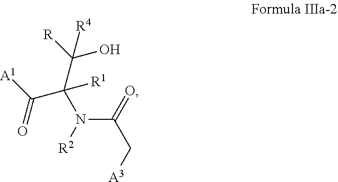

wherein A³ is the rest of the target protein.

In some embodiments, the compound/target protein complex has the structure of Formula IIIa-3:

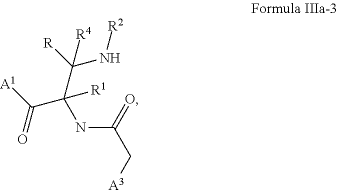

wherein A³ is the rest of the target protein.

In some embodiments, the compound/target protein complex has the structure of Formula IIIa-4:

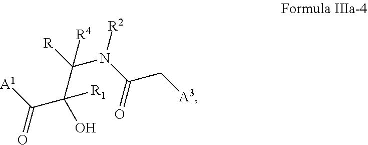

wherein A³ is the rest of the target protein.

In some embodiments, the compound/target protein complex has the structure of/target protein complex of claim 16, wherein the compound/target protein complex has the structure of Formula IIIb-1:

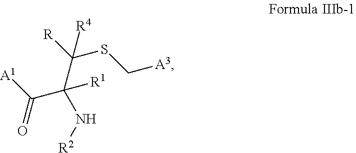

wherein A³ is the rest of the target protein.

In some embodiments, the compound/target protein complex has the structure of Formula IIIb-2:

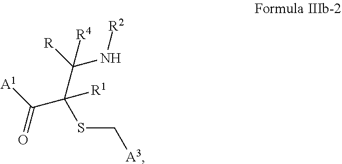

wherein A³ is the rest of the target protein.

In some embodiments, the compound/target protein complex has the structure of Formula IIIc-1:

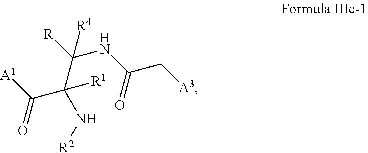

wherein A³ is the rest of the target protein.

In some embodiments, the compound/target protein complex has the structure of Formula IIIc-2:

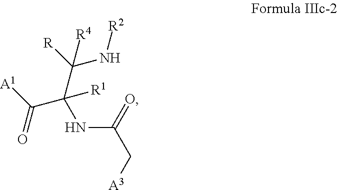

wherein A³ is the rest of the target protein.

In some embodiments, the compound/target protein complex has the structure of Formula IIIc-3:

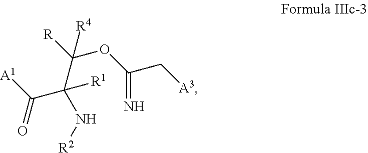

wherein A³ is the rest of the target protein.

In some embodiments, the compound/target protein complex has the structure of Formula IIIc-4:

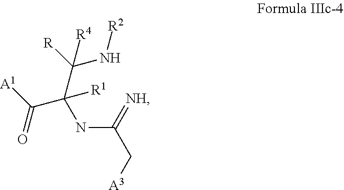

wherein A³ is the rest of the target protein.

In some embodiments, the compound/target protein complex has the structure of Formula IIId-1:

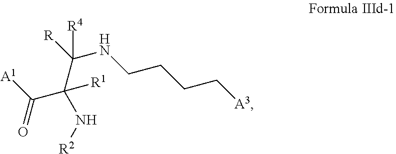

wherein A³ is the rest of the target protein.

In some embodiments, the compound/target protein complex has the structure of Formula IIId-2:

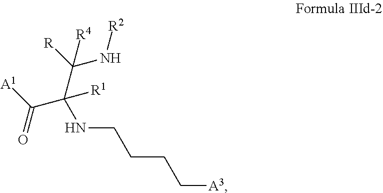

wherein A³ is the rest of the target protein.

In some embodiments, the compound/target protein complex has the structure of Formula IIIe-1:

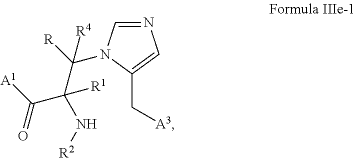

wherein A³ is the rest of the target protein.

In some embodiments, the compound/target protein complex has the structure of Formula IIIe-1:

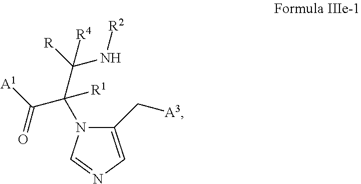

wherein A³ is the rest of the target protein.

In some embodiments, R is optionally substituted C₃-C₁₀ cycloalkyl, optionally substituted C₆-C₁₀ aryl, optionally substituted C₂-C₆ alkenyl, or optionally substituted C₂-C₆ alkynyl. In some embodiments, R is optionally substituted C₃-C₁₀ cycloalkyl. In some embodiments, R is optionally substituted cyclopropyl.

In some embodiments, R is

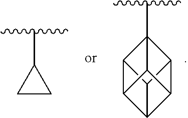

In some embodiments, R is

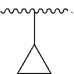 [SMILES: *C1CC1]

In some embodiments, R is optionally substituted C₂-C₆ alkenyl, or optionally substituted C₂-C₆ alkynyl. In some embodiments, R is

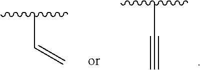

In some embodiments, R is a carbocation-stabilizing electron-donating group.

### Tri-Complexes

The vast majority of small molecule drugs act by binding a functionally important site on a target protein, thereby modulating (e.g., positively or negatively modulating) the activity of that protein. For example, the cholesterol-lowering drugs statins bind the enzyme active site of HMG-CoA reductase, thus preventing the enzyme from engaging with its substrates. The fact that many such drug/target interacting pairs are known may have misled some into believing that a small molecule modulator could be discovered for most, if not all, proteins provided a reasonable amount of time, effort, and resources. This is far from the case. Current estimates hold that only about 10% of all human proteins are targetable by small molecules. The other 90% are currently considered refractory or intractable toward small molecule drug discovery. Such targets are commonly referred to as “undruggable.” These undruggable targets include a vast and largely untapped reservoir of medically important human proteins. Thus, there exists a great deal of interest in discovering new molecular modalities capable of modulating the function of such undruggable targets.

The present invention encompasses the recognition that small molecules are typically limited in their targeting ability because their interactions with the target are driven by adhesive forces, the strength of which is roughly proportional to contact surface area. Because of their small size, the only way for a small molecule to build up enough intermolecular contact surface area to effectively interact with a target protein is to be literally engulfed by that protein. Indeed, a large body of both experimental and computational data supports the view that only those proteins having a hydrophobic “pocket” on their surface are capable of binding small molecules. In those cases, binding is enabled by engulfment. Not a single example exists of a small molecule binding with high-affinity to a protein outside of a hydrophobic pocket.

Nature has evolved a strategy that allows a small molecule to interact with target proteins at sites other than hydrophobic pockets. This strategy is exemplified by the naturally occurring immunosuppressive drugs cyclosporine A, rapamycin, and FK506. The activity of these drugs involves the formation of a high-affinity complex of the small molecule with a small presenting protein. The composite surface of the small molecule and the presenting protein then engages the target. Thus, for example, the binary complex formed between cyclosporine A and cyclophilin A targets calcineurin with high affinity and specificity, but neither cyclosporine A nor cyclophilin A alone binds calcineurin with measurable affinity.

Many important therapeutic targets exert their function by complexation with other proteins. The protein/protein interaction surfaces in many of these systems contain an inner core of hydrophobic side chains surrounded by a wide ring of polar residues. The hydrophobic residues contribute nearly all of the energetically favorable contacts, and hence this cluster has been designated as a “hotspot” for engagement in protein-protein interactions. Importantly, in the aforementioned complexes of naturally occurring small molecules with small presenting proteins, the small molecule provides a cluster of hydrophobic functionality akin to a hotspot, and the protein provides the ring of mostly polar residues. In other words, presented small molecule systems mimic the surface architecture employed widely in natural protein/protein interaction systems.

Compounds (e.g., macrocyclic compounds) of the invention are capable of modulating biological processes, for example through binding to a presenter protein (e.g., a member of the FKBP family, a member of the cyclophilin family, or PIN1) to form a presenter protein/compound complex as described above which binds to a target protein to form a tri-complex. The formation of these tri-complexes allows for modulation of proteins that do not have traditional binding pockets and/or are considered undruggable. The presenter protein/compound complexes are able to modulate biological processes through cooperative binding between the compound and the presenter protein. Both the compound and presenter protein have low affinity for the target protein alone, but the presenter protein/compound complex has high affinity for the target protein. Cooperative binding can be determined by measurement of the buried surface area of the target protein that includes atoms from the compound and/or presenter protein and/or by measurement of the free binding energy contribution of the compound and/or presenter protein. Binding is considered cooperative if at least one atom from each of the compound and presenter protein participates in binding with the target protein.

The binding of a presenter protein/compound complex and a target protein is achieved through formation of a combined binding site including residues from both the presenter protein and compound that allow for increased affinity that would not be possible with either the presenter protein or compound alone. For example at least 20% (e.g., at least 25%, at least 30%, at least 35%, at least 40%, at least 45%, at least 50%) of the total buried surface area of the target protein in the tri-complex includes one or more atoms that participate in binding to the compound and/or at least 20% (e.g., at least 25%, at least 30%, at least 35%, at least 40%, at least 45%, at least 50%) of the total buried surface area of the target protein in the tri-complex includes one or more atoms that participate in binding to the presenter protein. Alternatively, the compound contributes at least 10% (e.g., at least 20%, at least 25%, at least 30%, at least 35%, at least 40%, at least 45%, at least 50%, at least 60%, at least 70%, at least 80%, at least 90%) of the total binding free energy of the tri-complex and/or the presenter protein contributes at least 10% (e.g., at least 20% at least 25%, at least 30%, at least 35%, at least 40%, at least 45%, at least 50%, at least 60%, at least 70%, at least 80%, at least 90%) of the total binding free energy of the tri-complex.

In some embodiments, a presenter protein/compound complex binds at a flat surface site on a target protein. In some embodiments, a compound (e.g., macrocyclic compound) in a presenter protein/compound complex binds at a hydrophobic surface site on a target protein, e.g., a site that includes at least 50% hydrophobic residues. In some embodiments, at least 70% of the binding interactions between one or more of the atoms of a compound and one or more atoms of a target protein are van der Waals and/or π-effect interactions. In certain embodiments, a presenter protein/compound complex binds to a target protein at a site of a naturally occurring protein-protein interaction between a target protein and a protein that specifically binds the target protein. In some embodiments, a presenter protein/compound complex does not bind at an active site of a target protein. In some embodiments, a presenter protein/compound complex binds at an active site of a target protein.

A characteristic of compounds of the invention that form tri-complexes with a presenter protein and a target protein is a lack of major structural reorganization in the presenter protein/compound complex compared to the tri-complex. This lack of major structural reorganization results in a low entropic cost to reorganize into a configuration favorable for the formation of the tri-complex once the presenter protein/compound complex has been formed. For example, threshold quantification of RMSD can be measured using the align command in PyMOL version 1.7rc1 (Schrödinger LLC). Alternatively, RMSD can be calculated using the ExecutiveRMS parameter from the algorithm LigAlign (J. Mol. Graphics and Modelling 2010, 29, 93-101). In some embodiments, the structural organization of the compound (i.e., the average three-dimensional configuration of the atoms and bonds of the molecule) is substantially unchanged in the tri-complex compared to the compound when in the presenter protein/compound complex before binding to the target protein. For example, the root mean squared deviation (RMSD) of the two aligned structures is less than 1.

### Pharmaceutical Compositions and Methods of Use

One embodiment of the present invention provides pharmaceutical compositions containing a compound of the invention or a pharmaceutically acceptable salt thereof, and a pharmaceutically acceptable excipient, as well as methods of using the compounds of the invention to prepare such compositions.

As used herein, the term “pharmaceutical composition” refers to a compound, such as a compound of the present invention, or a pharmaceutically acceptable salt thereof, formulated together with a pharmaceutically acceptable excipient.

In some embodiments, a compound is present in a pharmaceutical composition in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

A “pharmaceutically acceptable excipient,” as used herein, refers to any inactive ingredient (for example, a vehicle capable of suspending or dissolving the active compound) having the properties of being nontoxic and non-inflammatory in a subject. Typical excipients include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

Compounds described herein, whether expressly stated or not, may be provided or utilized in salt form, e.g., a pharmaceutically acceptable salt form, unless expressly stated to the contrary. The term “pharmaceutically acceptable salt,” as use herein, refers to those salts of the compounds described herein that are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The compounds of the invention may have ionizable groups so as to be capable of preparation as pharmaceutically acceptable salts. These salts may be acid addition salts involving inorganic or organic acids or the salts may, in the case of acidic forms of the compounds of the invention, be prepared from inorganic or organic bases. In some embodiments, the compounds are prepared or used as pharmaceutically acceptable salts prepared as addition products of pharmaceutically acceptable acids or bases. Suitable pharmaceutically acceptable acids and bases are well-known in the art, such as hydrochloric, sulfuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-optionally substituted hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium, barium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

As used herein, the term “subject” refers to any member of the animal kingdom. In some embodiments, “subject” refers to humans, at any stage of development. In some embodiments, “subject” refers to a human patient. In some embodiments, “subject” refers to non-human animals. In some embodiments, the non-human animal is a mammal (e.g., a rodent, a mouse, a rat, a rabbit, a monkey, a dog, a cat, a sheep, cattle, a primate, or a pig). In some embodiments, subjects include, but are not limited to, mammals, birds, reptiles, amphibians, fish, or worms. In some embodiments, a subject may be a transgenic animal, genetically-engineered animal, or a clone.

As used herein, the term “dosage form” refers to a physically discrete unit of a compound (e.g., a compound of the present invention) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms. As used herein, the term “dosing regimen” refers to a set of unit doses (typically more than one) that are administered individually to a subject, typically separated by periods of time. In some embodiments, a given therapeutic compound (e.g., a compound of the present invention) has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

A “therapeutic regimen” refers to a dosing regimen whose administration across a relevant population is correlated with a desired or beneficial therapeutic outcome.

The term “treatment” (also “treat” or “treating”), in its broadest sense, refers to any administration of a substance (e.g., a compound of the present invention) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder or condition or of a subject who exhibits only early signs of the disease, disorder, or condition. Alternatively, or additionally, in some embodiments, treatment may be administered to a subject who exhibits one or more established signs of the relevant disease, disorder or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

For use as treatment of subjects, the compounds of the invention, or a pharmaceutically acceptable salt thereof, can be formulated as pharmaceutical or veterinary compositions. Depending on the subject to be treated, the mode of administration, and the type of treatment desired, e.g., prevention, prophylaxis, or therapy, the compounds, or a pharmaceutically acceptable salt thereof, are formulated in ways consonant with these parameters. A summary of such techniques may be found in Remington: The Science and Practice of Pharmacy, 21^{st} Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

Compositions can be prepared according to conventional mixing, granulating or coating methods, respectively, and the present pharmaceutical compositions can contain from about 0.1% to about 99%, from about 5% to about 90%, or from about 1% to about 20% of a compound of the present invention, or pharmaceutically acceptable salt thereof, by weight or volume. In some embodiments, compounds, or a pharmaceutically acceptable salt thereof, described herein may be present in amounts totaling 1-95% by weight of the total weight of a composition, such as a pharmaceutical composition.

The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

As used herein, the term “administration” refers to the administration of a composition (e.g., a compound, or a preparation that includes a compound as described herein) to a subject or system. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal or vitreal.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. A formulation will generally include a diluent as well as, in some cases, adjuvants, buffers, preservatives and the like. Compounds, or a pharmaceutically acceptable salt thereof, can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the invention, or a pharmaceutically acceptable salt thereof. Suitable forms include syrups, capsules, and tablets, as is understood in the art.

Each compound, or a pharmaceutically acceptable salt thereof, as described herein, may be formulated in a variety of ways that are known in the art. For example, the first and second agents of the combination therapy may be formulated together or separately. Other modalities of combination therapy are described herein.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dose or in which the individual compounds, or a pharmaceutically acceptable salt thereof, may vary in potency as therapy progresses); or the kit may contain multiple doses suitable for administration to multiple subjects (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, optionally substituted hydroxylpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release can be achieved by appropriate coating of a tablet, capsule, pellet, or granulate formulation of compounds, or by incorporating the compound, or a pharmaceutically acceptable salt thereof, into an appropriate matrix. A controlled release coating may include one or more of the coating substances mentioned above or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-optionally substituted hydroxylmethacrylate, methacrylate hydrogels, 1,3 butylene glycol, ethylene glycol methacrylate, or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, or halogenated fluorocarbon.

The liquid forms in which the compounds, or a pharmaceutically acceptable salt thereof, and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the compounds of the invention, or a pharmaceutically acceptable salt thereof, will depend on the nature of the compound, and can readily be determined by one skilled in the art. A dosage may be, for example, about 0.001 mg to about 2000 mg per day, about 1 mg to about 1000 mg per day, about 5 mg to about 500 mg per day, about 100 mg to about 1500 mg per day, about 500 mg to about 1500 mg per day, about 500 mg to about 2000 mg per day, or any range derivable therein. In some embodiments, the daily dose range for oral administration, for example, may lie within the range of from about 0.001 mg to about 2000 mg per kg body weight of a human, in single or divided doses. On the other hand, it may be necessary to use dosages outside these limits in some cases.

In some embodiments, the pharmaceutical composition may further comprise an additional compound having antiproliferative activity. Depending on the mode of administration, compounds, or a pharmaceutically acceptable salt thereof, will be formulated into suitable compositions to permit facile delivery. Each compound, or a pharmaceutically acceptable salt thereof, of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

It will be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics or procedures and the desired therapeutic effect to be achieved. It will also be appreciated that the therapies employed may achieve a desired effect for the same disorder, or they may achieve different effects (e.g., control of any adverse effects).

Administration of each drug in a combination therapy, as described herein, can, independently, be one to four times daily for one day to one year, and may even be for the life of the subject. Chronic, long-term administration may be indicated.

### Methods of Use

The compounds and complexes of the present disclosure may be useful in various methods. For example, the present disclosure provides a method of modulating a target protein by contacting the target protein with a compound or presenter protein/compound complex described herein. The method may include inhibiting a target protein by contacting the target protein with a compound or presenter protein/compound complex described herein or activating a target protein by contacting the target protein with a compound or presenter protein/compound complex described herein. The modulating (e.g., inhibiting or activating) may render the compounds and/or complexes useful for the treatment of various diseases or disorders. Accordingly, the compounds and complexes may be used in a method of treating a disease or disorder in a subject in need thereof by administering to the subject a therapeutically effective amount of a compound described herein. In some embodiments, the subject has previously been treated with a prior therapy. In some embodiments, the subject has developed resistance to treatment with a prior therapy.

The present disclosure also relates to the synthesis of the various complexes disclosed. For example, the disclosure provides a method of forming a tri-complex described herein by contacting a target protein with a presenter protein/compound complex described herein.

In some embodiments of the foregoing methods, upon contacting the target protein, the target protein forms a covalent bond to the compound or the presenter protein/compound complex. In some embodiments, upon contacting the target protein, an aspartic acid, glutamic acid, cysteine, glutamine, asparagine, lysine, or histidine residue of the target protein forms a covalent bond to the compound or the complex. In some embodiments, upon contacting the target protein, an aspartic acid, glutamic acid, cysteine, glutamine, or asparagine residue of the target protein forms a covalent bond to the compound or the complex.

In an embodiment, the disclosure provides a method of crosslinking a compound described herein to a second moiety by contacting the second moiety with the compound under conditions sufficient to form a covalent bond between the compound and the second moiety. In some embodiments, the second moiety is a target protein.

The disclosure further provides a method of forming a presenter protein/compound complex described herein by contacting a presenter protein with a compound described herein under conditions sufficient to permit the formation of a complex. In some embodiments, the complex is formed by way of noncovalent interactions.

In another embodiment, the disclosure provides a method of forming a tri-complex described herein, including the following steps:
a) contacting a presenter protein with the compound described herein under conditions sufficient to permit the formation of presenter protein/compound complex; and
b) contacting the presenter protein/compound complex with a target protein under conditions that permit the formation of a tri-complex.

In some embodiments, the presenter protein/compound complex binds to the target protein with at least 5-fold greater affinity than the presenter protein or the compound alone. In some embodiments, the presenter protein or the compound do not substantially bind to the target protein in the absence of forming the presenter protein/compound complex.

Compounds of the present invention may be used in methodologies including click chemistry. Persons having skill in the art would be familiar with how to adapt compounds disclosed herein for click chemistry applications. See, e.g., Jewett et al., J. Am. Chem. Soc. 2010, 132, 3688-3690; Gui et al., ChemRxiv 2022, 1-11; and Scinto et al., Nature Reviews Methods Primers 2021, 1, 1-23. Compounds of the present invention are also adaptable for uses in antibody-drug conjugates as well as degrader applications. Incorporation of the aziridine moieties described herein into known modulators (e.g., RAS inhibitors) are also contemplated. Exemplary scaffolds that are amenable to such modification include known RAS scaffolds and compounds disclosed in the art, such as WO 2022152233, WO 2022148422, WO 2022148421, WO 2022135346, WO 2022133731, WO 2022133038, WO 2022133345, WO 2022132200, WO 2022119748, WO 2022109487, WO 2022109485, WO 2022105859, WO 2022105857, WO 2022098625, WO 2022098625, WO 2022093856, WO 2022087335, WO 2022083569, WO 2022078470, WO 2022078414, WO 2022072783, WO 2022066805, WO 2022066646, WO 2022048545, WO 2022047093, WO 2022042630, WO 2022031678, WO 2022028492, WO 2022015375, WO 2022002102, WO 2021190467, WO 2021185233, WO 2021180181, WO 2021175199, 2021173923, WO 2021169990, WO 2021169963, WO 2021168193, WO 2021158071, WO 2021155716, WO 2021152149, WO 2021150613, WO 2021147967, WO 2021147965, WO 2021143693, WO 2021142252, WO 2021141628, WO 2021139748, WO 2021139678, WO 2021129824, WO 2021129820, WO 2021127404, WO 2021126816, WO 2021126799, WO 2021124222, WO 2021121371, WO 2021121367, WO 2021121330, WO 2021108643, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018217651, WO 2018218071, WO 2018218069, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659, WO 2013155223, and PCT/US2022/027773, each of which is incorporated by reference in its entirety.

Compounds of the present invention may be used in the treatment of subjects having a disease or disorder, such as mammals (e.g., mice, rats, dogs, and humans). The disease or disorder may be, for example, cancer, diabetes, cardiovascular disease, neurological disorder, viral disease, infectious disease, autoimmune disease, arthritis, an allergic disorder, inflammation, a hormone-related disease, a condition associated with organ transplantation (e.g., transplant rejection), an immunodeficiency disorder, a bone disorder, or a proliferative disorder.

### Kits

The invention also features kits including (a) a pharmaceutical composition including an agent (e.g., a compound or complex of the invention) described herein, and (b) a package insert with instructions to perform any of the methods described herein. In some embodiments, the kit includes (a) a pharmaceutical composition including an agent (e.g., a compound or complex of the invention) described herein, (b) one or more additional therapies (e.g., non-drug treatment or therapeutic agent), and (c) a package insert with instructions to perform any of the methods described herein.

As one aspect of the present invention contemplates the treatment of the disease or symptoms associated therewith with a combination of pharmaceutically active compounds that may be administered separately, the invention further relates to combining separate pharmaceutical compositions in kit form. The kit may comprise two separate pharmaceutical compositions: a compound of the present invention, and one or more additional therapies. The kit may comprise a container for containing the separate compositions such as a divided bottle or a divided foil packet. Additional examples of containers include syringes, boxes, and bags. In some embodiments, the kit may comprise directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

### ENUMERATED EMBODIMENTS

Some specific embodiments are listed below. The below enumerated embodiments should not be construed to limit the scope of the disclosure, rather, the below are presented as some examples of the utility of the disclosure.

1. A compound having the structure of Formula I:

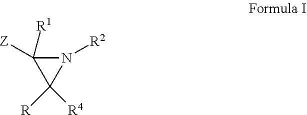

or a pharmaceutically acceptable salt thereof; wherein
M⁺ is a cation;

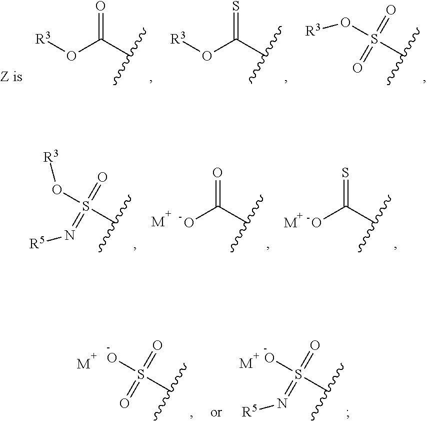

R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R and R¹ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R and R² combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; and R¹ and R⁴ are each, independently, hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a}); or
R and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃—C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R² combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and each of R and R⁴ is, independently, hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R² and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and each of R¹ and R is, independently, hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
wherein at least one of R and R⁴ is not hydrogen;
R³ is hydrogen, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₃-C₁₀ cycloalkyl, optionally substituted C₃-C₁₀ cycloalkenyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl;
each R^{1a}, R^{2a}, R^{2c}, R^{2d}, and R^{2e} is, independently, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl;
R^{2b} is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁵ is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl,
and wherein each hydrogen is independently, optionally, isotopically enriched for deuterium.

2. The compound of embodiment 1, or a pharmaceutically acceptable salt thereof, wherein the compound has the structure of Formula Ia:

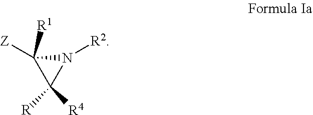

3. The compound of embodiment 1, or a pharmaceutically acceptable salt thereof, wherein the compound has the structure of Formula Ib:

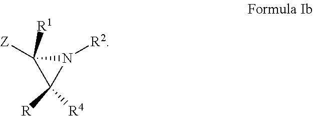

4. The compound of embodiment 1, or a pharmaceutically acceptable salt thereof, wherein the compound has the structure of Formula Ic:

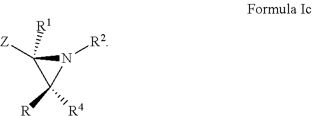

5. The compound of embodiment 1, or a pharmaceutically acceptable salt thereof, wherein the compound has the structure of Formula Id:

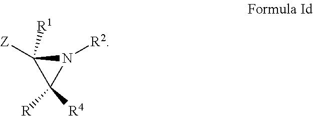

6. The compound of any one of embodiments 1 to 5, or a pharmaceutically acceptable salt thereof, wherein R³ is optionally substituted C₁-C₆ alkyl, optionally substituted 3- to 10-membered heterocycloalkyl, optionally substituted C₆-C₁₀ aryl, or optionally substituted 5- to 10-membered heteroaryl.

7. The compound of any one of embodiments 1 to 5, or a pharmaceutically acceptable salt thereof, wherein R³ is optionally substituted C₁-C₆ alkyl.

8. The compound of any one of embodiments 1 to 5, or a pharmaceutically acceptable salt thereof, wherein R³ is methyl, ethyl, or benzyl.

9. The compound of any one of embodiments 1 to 5, or a pharmaceutically acceptable salt thereof, wherein M⁺ is Li⁺.

10. The compound of any one of embodiments 1 to 5, or a pharmaceutically acceptable salt thereof, wherein R³ is:

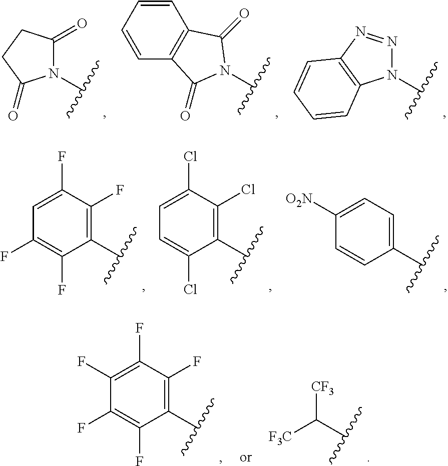

11. The compound of any one of embodiments 1 to 10, or a pharmaceutically acceptable salt thereof, wherein Z is

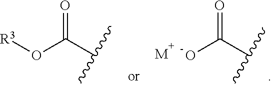

12. A compound having the structure of Formula II:

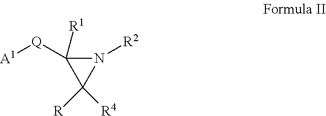

or a pharmaceutically acceptable salt thereof,
wherein A¹ is a monovalent organic moiety;
Q is

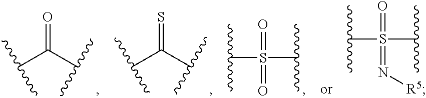

R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
or R and R¹ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R and R² combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and R¹ and R⁴ are each, independently, hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a}); or
R and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R² combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and each of R and R⁴ is, independently, hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R² and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and each of R¹ and R is, independently, hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
wherein at least one of R and R⁴ is not hydrogen;
each R^{1a}, R^{2a}, R^{2c}, R^{2d}, and R^{2e} is, independently, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl;
R^{2b} is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁵ is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl,
and wherein each hydrogen is independently, optionally, isotopically enriched for deuterium.

13. The compound of embodiment 12, or a pharmaceutically acceptable salt thereof, wherein the compound has the structure of Formula IIa:

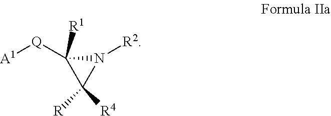

14. The compound of embodiment 12, or a pharmaceutically acceptable salt thereof, wherein the compound has the structure of Formula IIb:

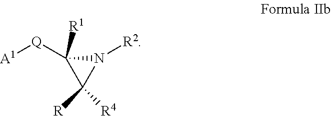

15. The compound of embodiment 12, or a pharmaceutically acceptable salt thereof, wherein the compound has the structure of Formula IIc:

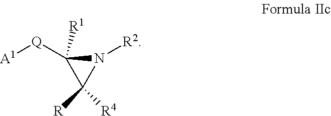

16. The compound of embodiment 12, or a pharmaceutically acceptable salt thereof, wherein the compound has the structure of Formula IId:

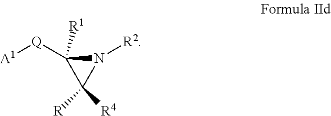

17. The compound of any one of embodiments 12 to 16, or a pharmaceutically acceptable salt thereof, wherein Q is

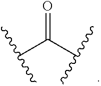 [SMILES: *C(*)=O]

18. A compound/target protein complex, or a pharmaceutically acceptable salt thereof, wherein the compound/target protein complex has the structure of Formula IIIa or Formula IIIb:

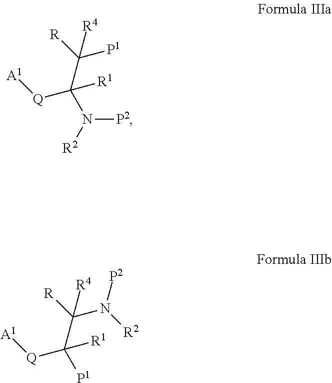

wherein A¹ is a monovalent organic moiety;
Q is

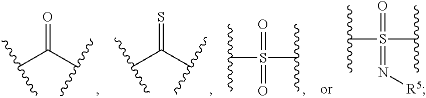

P¹ is A², and P² is hydrogen; or P¹ is hydroxyl, and P² is A²;
A² is the target protein;
R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl or —Si(R^{1a})₃; or
R and R¹ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl, and R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R and R² combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and R¹ and R⁴ are each, independently, hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a}); or
R and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R² combine to form an optionally substituted C₃-C₁₀ cycloalkyl, optionally substituted C₃-C₁₀ cycloalkenyl, optionally substituted C₈-C₁₀ cycloalkynyl, or optionally substituted 3- to 10-membered heterocycloalkyl, optionally substituted 5- to 10-membered heterocycloalkenyl, or optionally substituted 8- to 10-membered heterocycloalkynyl, and each of R and R⁴ is, independently, hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl or —Si(R^{1a})₃; or
R² and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and each of R¹ and R is, independently, hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
wherein at least one of R and R⁴ is not hydrogen;
each R¹—, R²³, R^{2c}, R^{2d}, and R^{2e} is, independently, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl;
R^{2b} is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted 5- to 10-membered heterocycloalkenyl, optionally substituted 8- to 10-membered heterocycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁵ is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl,
and wherein each hydrogen is independently, optionally, isotopically enriched for deuterium.

19. The compound/target protein complex of embodiment 18, wherein the compound/target protein complex has the structure of Formula IIIa-1:

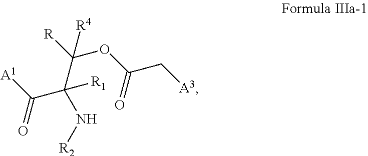

wherein A³ is the rest of the target protein.

20. The compound/target protein complex of embodiment 18, wherein the compound/target protein complex has the structure of Formula IIIa-2:

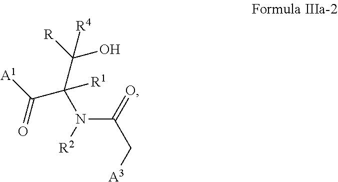

wherein A³ is the rest of the target protein.

21. The compound/target protein complex of embodiment 18, wherein the compound/target protein complex has the structure of Formula IIIa-3:

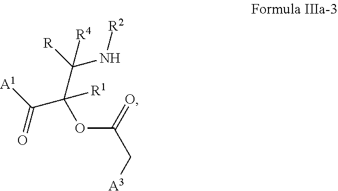

wherein A³ is the rest of the target protein.

22. The compound/target protein complex of embodiment 18, wherein the compound/target protein complex has the structure of Formula IIIa-4:

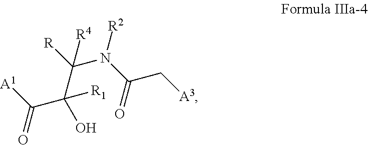

wherein A³ is the rest of the target protein.

23. The compound/target protein complex of embodiment 18, wherein the compound/target protein complex has the structure of Formula IIIb-1:

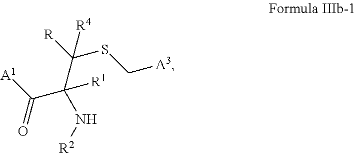

wherein A³ is the rest of the target protein.

24. The compound/target protein complex of embodiment 18, wherein the compound/target protein complex has the structure of Formula IIIb-2:

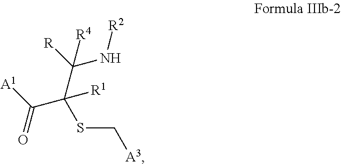

wherein A³ is the rest of the target protein.

25. The compound/target protein complex of embodiment 18, wherein the compound/target protein complex has the structure of Formula IIIc-1:

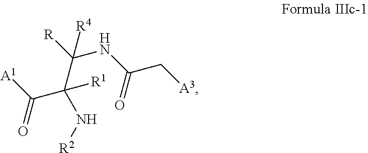

wherein A³ is the rest of the target protein.

26. The compound/target protein complex of embodiment 18, wherein the compound/target protein complex has the structure of Formula IIIc-2:

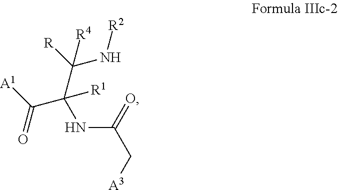

wherein A³ is the rest of the target protein.

27. The compound/target protein complex of embodiment 18, wherein the compound/target protein complex has the structure of Formula IIIc-3:

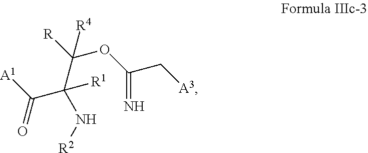

wherein A³ is the rest of the target protein.

28. The compound/target protein complex of embodiment 18, wherein the compound/target protein complex has the structure of Formula IIIc-4:

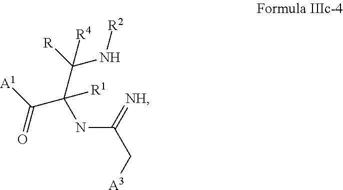

wherein A³ is the rest of the target protein.

29. The compound/target protein complex of embodiment 18, wherein the compound/target protein complex has the structure of Formula IIId-1:

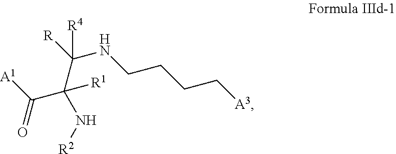

wherein A³ is the rest of the target protein.

30. The compound/target protein complex of embodiment 18, wherein the compound/target protein complex has the structure of Formula IIId-2:

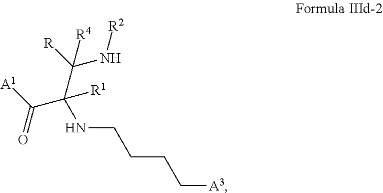

wherein A³ is the rest of the target protein.

31. The compound/target protein complex of embodiment 18, wherein the compound/target protein complex has the structure of Formula IIIe-1:

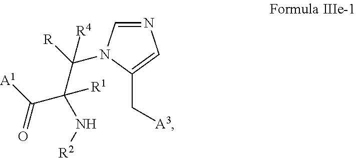

wherein A³ is the rest of the target protein.

32. The compound/target protein complex of embodiment 18, wherein the compound/target protein complex has the structure of Formula IIIe-2:

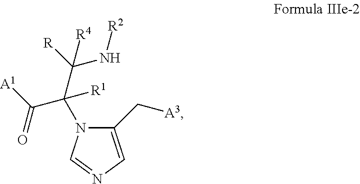

wherein A³ is the rest of the target protein.

33. The compound of any one of embodiments 1 to 17, or a pharmaceutically acceptable salt thereof, or the compound/target protein complex of any one of embodiments 18 to 33, wherein R is optionally substituted C₃-C₁₀ cycloalkyl, optionally substituted C₆-C₁₀ aryl, optionally substituted C₂-C₆ alkenyl, or optionally substituted C₂-C₆ alkynyl.

34. The compound or compound/target protein complex of embodiment 33, or a pharmaceutically acceptable salt thereof, wherein R is optionally substituted C₃-C₁₀ cycloalkyl

35. The compound or compound/target protein complex of embodiment 34, or a pharmaceutically acceptable salt thereof, wherein R is optionally substituted cyclopropyl.

36. The compound or compound/target protein complex of embodiment 35, or a pharmaceutically acceptable salt thereof, wherein R is

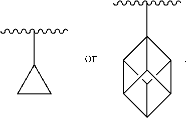

37. The compound or compound/target protein complex of embodiment 36, or a pharmaceutically acceptable salt thereof, wherein R is

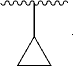 [SMILES: *C1CC1]

38. The compound or compound/target protein complex of embodiment 33, or a pharmaceutically acceptable salt thereof, wherein R is optionally substituted C₂-C₆ alkenyl, or optionally substituted C₂-C₆ alkynyl.

39. The compound or compound/target protein complex of embodiment 38, or a pharmaceutically acceptable salt thereof, wherein R is

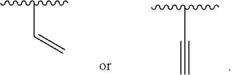

40. The compound of any one of embodiments 1 to 17, or a pharmaceutically acceptable salt thereof, or the compound/target protein complex of any one of embodiments 18 to 33, wherein R is a carbocation-stabilizing electron-donating group.

41. The compound or compound/target protein complex of any one of embodiments 1 to 40, or a pharmaceutically acceptable salt thereof, wherein R¹ is hydrogen.

42. The compound or compound/target protein complex of any one of embodiments 1 to 41, or a pharmaceutically acceptable salt thereof, wherein R² is hydrogen, optionally substituted C₁-C₆ alkyl, optionally substituted C₁-C₆ heteroalkyl, optionally substituted C₃-C₁₀ cycloalkyl, or optionally substituted 3- to 10-membered heterocycloalkyl.

43. The compound or compound/target protein complex of embodiment 42, or a pharmaceutically acceptable salt thereof, wherein R² is optionally substituted C₁-C₆ alkyl, optionally substituted C₁-C₆ heteroalkyl, optionally substituted C₃-C₁₀ cycloalkyl, or optionally substituted 3- to 10-membered heterocycloalkyl.

44. The compound or compound/target protein complex of embodiment 43, or a pharmaceutically acceptable salt thereof, wherein R² is optionally substituted C₁-C₆ alkyl.

45. The compound or compound/target protein complex of embodiment 43, or a pharmaceutically acceptable salt thereof, wherein R² is optionally substituted C₃-C₁₀ cycloalkyl.

46. The compound or compound/target protein complex of embodiment 43, or a pharmaceutically acceptable salt thereof, wherein R² is optionally substituted 3- to 10-membered heterocycloalkyl.

47. The compound or compound/target protein complex of any one of embodiments 1 to 41, or a pharmaceutically acceptable salt thereof, wherein R² is:

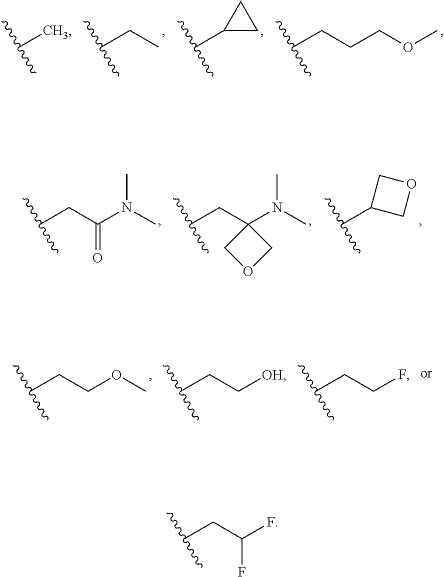

48. The compound or compound/target protein complex of embodiment 47, or a pharmaceutically acceptable salt thereof, wherein R² is:

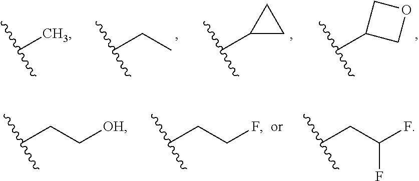

49. The compound or compound/target protein complex of embodiment 48, or a pharmaceutically acceptable salt thereof, wherein R² is methyl.

50. The compound or compound/target protein complex of any one of embodiments 1 to 49, or a pharmaceutically acceptable salt thereof, wherein R⁴ is hydrogen.

51. The compound or compound/target protein complex of any one of embodiments 12 to 50, or a pharmaceutically acceptable salt thereof, wherein A¹ is a protein.

52. The compound or compound/target protein complex of any one of embodiments 12 to 50, or a pharmaceutically acceptable salt thereof, wherein A¹ is a nucleic acid.

53. The compound or compound/target protein complex of any one of embodiments 12 to 50, or a pharmaceutically acceptable salt thereof, wherein A¹ is a small molecule.

54. The compound or compound/target protein complex of any one of embodiments 12 to 50, or a pharmaceutically acceptable salt thereof, wherein A¹ is a macrocyclic small molecule.

55. The compound or compound/target protein complex of any one of embodiments 12 to 50, or a pharmaceutically acceptable salt thereof, wherein A¹ has the structure of Formula IV:

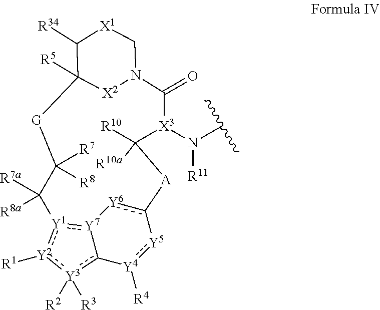

wherein the dotted lines represent zero, one, two, three, or four non-adjacent double bonds;
A is —N(H or CH₃)C(O)—(CH₂)—where the amino nitrogen is bound to the carbon atom of —CH(R¹⁰)—, optionally substituted C₂-C₄ alkylene, optionally substituted C₁-C₄ heteroalkylene, or optionally substituted C₂-C₄ alkenylene, optionally substituted 3- to 6-membered cycloalkylene, optionally substituted 3- to 6-membered heterocycloalkylene, optionally substituted 6-membered arylene, or optionally substituted 5- to 10-membered heteroarylene;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂ or N;
Y⁶ is C(O), CH, CH₂, or N;
R¹ is cyano, optionally substituted C₁-C₆ alkyl, optionally substituted C₁-C₆ heteroalkyl, optionally substituted 3- to 6-membered cycloalkyl, optionally substituted 3- to 6-membered cycloalkenyl, optionally substituted 3- to 6-membered heterocycloalkyl, optionally substituted 6 to 10-membered aryl, or optionally substituted 5- to 10-membered heteroaryl, or
R¹ and R² combine with the atoms to which they are attached to form an optionally substituted 3- to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3- to 6-membered cycloalkyl, optionally substituted 3- to 7-membered heterocycloalkyl, optionally substituted 6-membered aryl, optionally substituted 5- or 6-membered heteroaryl; R³ is absent, or R² and R³ combine with the atom to which they are attached to form an optionally substituted 3- to 8-membered cycloalkyl or optionally substituted 3- to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or R⁶ and R⁷ combine with the carbon atoms to which they are attached to form an optionally substituted 3- to 6-membered cycloalkyl or optionally substituted 3- to 7-membered heterocycloalkyl;
R⁸ is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6 to 10-membered aryl, or R⁷ and R⁸ combine with the carbon atom to which they are attached to form C═CR^{7′}R^{8′}; C═N(OH), C═N(O—C₁-C₃ alkyl), C═O, C═S, C═NH, optionally substituted 3- to 6-membered cycloalkyl, or optionally substituted 3- to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3- to 8-membered cycloalkyl, optionally substituted 3- to 14-membered heterocycloalkyl, optionally substituted 5- to 10-membered heteroaryl, or optionally substituted 6- to 10-membered aryl, or
R^{7′} and R^{8′} combine with the carbon atom to which they are attached to form optionally substituted 3- to 6-membered cycloalkyl or optionally substituted 3- to 7-membered heterocycloalkyl;
R⁹ is hydrogen, F, optionally substituted C₁-C₆ alkyl, optionally substituted C₁-C₆ heteroalkyl, optionally substituted 3- to 6-membered cycloalkyl, or optionally substituted 3- to 7-membered heterocycloalkyl, or
R⁹ and L combine with the atoms to which they are attached to form an optionally substituted 3- to 14-membered heterocycloalkyl; R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R³⁴ is hydrogen or C₁-C₃ alkyl, and wherein each hydrogen is independently, optionally, isotopically enriched for deuterium.

56. The compound or compound/target protein complex of any one of embodiments 12 to 50, or a pharmaceutically acceptable salt thereof, wherein A¹ has the structure of Formula V:

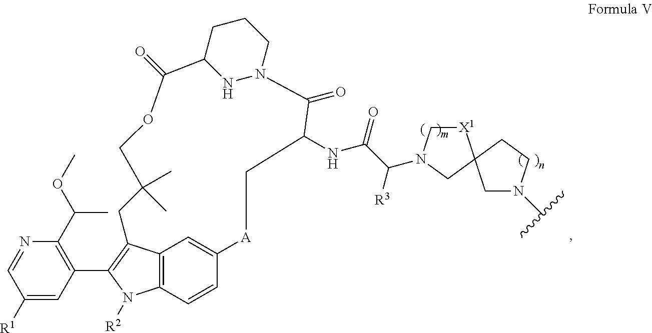

wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
X¹ is CH₂ or O;
m is 1 or 2;
n is 0 or 1;
R¹ is hydrogen or optionally substituted 3- to 10-membered heterocycloalkyl;
R² is optionally substituted C₁-C₆ alkyl; and
R³ is optionally substituted C₁-C₆ alkyl or optionally substituted 3- to 6-membered cycloalkyl, and wherein each hydrogen is independently, optionally, isotopically enriched for deuterium.

57. The compound or compound/target protein complex of any one of embodiments 12 to 50, or a pharmaceutically acceptable salt thereof, wherein A¹ has the structure of Formula VI:

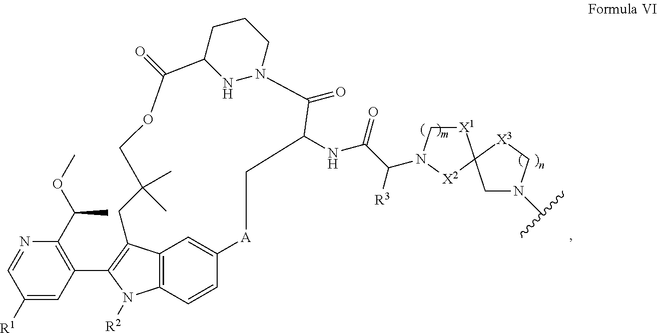

wherein A is optionally substituted 3- to 6-membered heterocycloalkylene, optionally substituted 3- to 6-membered cycloalkylene, optionally substituted 6-membered arylene, or optionally substituted 5- to 10-membered heteroarylene;
X¹, X², and X³ are each independently selected from CH₂, CHF, CF₂, C═O, or 0;
m is 1 or 2;
n is 0 or 1;
R¹ is hydrogen, optionally substituted C₁-C₆ heteroalkyl, or optionally substituted 3- to 10-membered heterocycloalkyl;
R² is optionally substituted C₁-C₆ alkyl; and
R³ is optionally substituted C₁-C₆ alkyl, optionally substituted C₁-C₆ heteroalkyl, optionally substituted 3- to 6-membered cycloalkyl, or optionally substituted heterocycloalkyl, and wherein each hydrogen is independently, optionally, isotopically enriched for deuterium.

58. The compound or compound/target protein complex of any one of embodiments 12 to 50, wherein A¹ has the structure of any one of Formula VII, VIII, and IX:

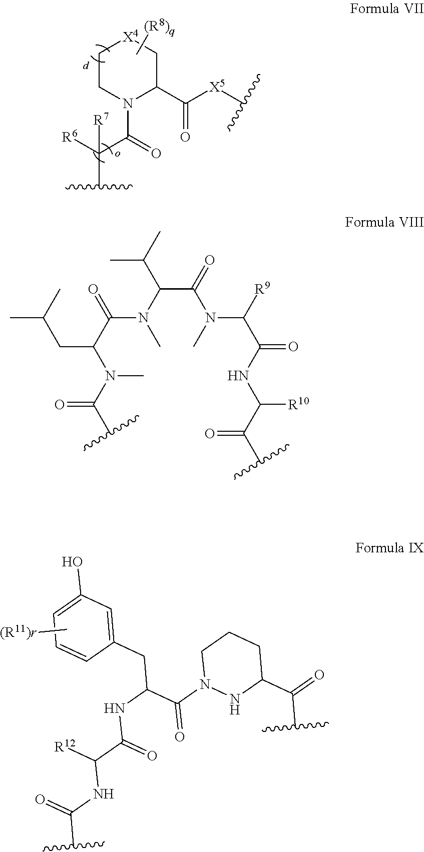

wherein o, and p are independently 0, 1, or 2;
q is an integer between 0 and 7;
r is an integer between 0 and 4;
X⁴ and X⁵ are each, independently, absent, CH₂, O, S, SO, SO₂, or NR¹¹;
each R⁶ and R⁷ are independently hydrogen, hydroxyl, optionally substituted amino, halogen, thiol, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted C₃-C₁₀ carbocyclyl, optionally substituted C₆-C₁₀ aryl, optionally substituted C₆-C₁₀ aryl C₁-C₆ alkyl, optionally substituted C₂-C₉ heteroaryl, optionally substituted C₂-C₉ heteroaryl C₁-C₆ alkyl, optionally substituted C₂-C₉ heterocyclyl, optionally substituted C₂-C₉ heterocyclyl C₁-C₆ alkyl, or R⁶ and R⁷ combine with the carbon atom to which they are bound to form C═O;
each R⁸ is, independently, hydroxyl, optionally substituted amino, halogen, thiol, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆heteroalkynyl, optionally substituted C₃-C₁₀ carbocyclyl, optionally substituted C₆-C₁₀ aryl, optionally substituted C₆-C₁₀ aryl C₁-C₆ alkyl, optionally substituted C₂-C₉ heteroaryl, optionally substituted C₂-C₉ heteroaryl C₁-C₆ alkyl, optionally substituted C₂-C₉ heterocyclyl, or optionally substituted C₂-C₉ heterocyclyl C₁-C₆ alkyl, or two R⁸ combine to form an optionally substituted C₃-C₁₀ carbocyclyl, optionally substituted C₆-C₁₀ aryl, or optionally substituted C₂-C₉ heteroaryl;
R⁹ is optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted C₃-C₁₀ carbocyclyl, optionally substituted C₆-C₁₀ aryl, optionally substituted C₆-C₁₀ aryl C₁-C₆ alkyl, optionally substituted C₂-C₉ heteroaryl, optionally substituted C₂-C₉ heteroaryl C₁-C₆ alkyl, optionally substituted C₂-C₉ heterocyclyl, or optionally substituted C₂-C₉ heterocyclyl C₁-C₆ alkyl;
R¹⁰ is optionally substituted C₁-C₆ alkyl;
each R¹¹ is, independently, hydroxyl, cyano, optionally substituted amino, halogen, thiol, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted C₃-C₁₀ carbocyclyl, optionally substituted C₆-C₁₀ aryl, optionally substituted C₆-C₁₀ aryl C₁-C₆ alkyl, optionally substituted C₂-C₉ heteroaryl, optionally substituted C₂-C₉ heteroaryl C₁-C₆ alkyl, optionally substituted C₂-C₉ heterocyclyl, or optionally substituted C₂-C₉ heterocyclyl C₁-C₆ alkyl; and
R¹² and R¹³ are each, independently, hydrogen, optionally substituted C₁-C₆ alkyl, optionally substituted C₂-C₆ alkenyl, optionally substituted C₂-C₆ alkynyl, optionally substituted aryl, C₃-C₇ carbocyclyl, optionally substituted C₆-C₁₀ aryl C₁-C₆ alkyl, and optionally substituted C₃-C₇ carbocyclyl C₁-C₆ alkyl,
and wherein each hydrogen is independently, optionally, isotopically enriched for deuterium.

59. A tri-complex comprising a presenter protein, a compound of any one of embodiments 12 to 58, and a target protein.

60. The compound/target protein complex of any one of embodiments 18 to 58, or the tri-complex of embodiment 59, wherein the target protein is a GTPase, GTPase activating protein, Guanine nucleotide-exchange factor, a heat shock protein, an ion channel, a coiled-coil protein, a kinase, a phosphatase, a ubiquitin ligase, a transcription factor, a chromatin modifier/remodeler, a protease, or a protein with classical protein-protein interaction domains and motifs.

61. The compound/target protein complex or tri-complex of embodiment 60, wherein the target protein is a GTPase selected from the group consisting of DIRAS1, DIRAS2, DIRAS3, ERAS, GEM, HRAS, KRAS, MRAS, NKIRAS1, NKIRAS2, NRAS, RALA, RALB, RAP1A, RAP1B, RAP2A, RAP2B, RAP2C, RASD1, RASD2, RASL10A, RASL10B, RASL11A, RASL11B, RASL12, REM1, REM2, RERG, RERGL, RRAD, RRAS, RRAS2, RHOA, RHOB, RHOBTB1, RHOBTB2, RHOBTB3, RHOC, RHOD, RHOF, RHOG, RHOH, RHOJ, RHOQ, RHOU, RHOV, RND1, RND2, RND3, RAC1, RAC2, RAC3, CDC42, RAB1A, RAB1B, RAB2, RAB3A, RAB3B, RAB3C, RAB3D, RAB4A, RAB4B, RABSA, RAB5B, RAB5C, RAB6A, RAB6B, RAB6C, RAB7A, RAB7B, RAB7L1, RAB8A, RAB8B, RAB9, RAB9B, RABL2A, RABL2B, RABL4, RAB10, RAB11A, RAB11B, RAB12, RAB13, RAB14, RAB15, RAB17, RAB18, RAB19, RAB20, RAB21, RAB22A, RAB23, RAB24, RAB25, RAB26, RAB27A, RAB27B, RAB28, RAB2B, RAB30, RAB31, RAB32, RAB33A, RAB33B, RAB34, RAB35, RAB36, RAB37, RAB38, RAB39, RAB39B, RAB40A, RAB40AL, RAB40B, RAB40C, RAB41, RAB42, RAB43, RAP1A, RAP1B, RAP2A, RAP2B, RAP2C, ARF1, ARF3, ARF4, ARF5, ARF6, ARL1, ARL2, ARL3, ARL4, ARL5, ARL5C, ARL6, ARL7, ARL8, ARL9, ARL10A, ARL10B, ARL10C, ARL11, ARL13A, ARL13B, ARL14, ARL15, ARL16, ARL17, TRIM23, ARL4D, ARFRP1, ARL13B, RAN, RHEB, RHEBL1, RRAD, GEM, REM, REM2, RIT1, RIT2, RHOT1, and RHOT2.

62. The compound/target protein complex or tri-complex of embodiment 61, wherein the target protein is a member of the RAS family.

63. The compound/target protein complex or tri-complex of embodiment 62, wherein the target protein is HRAS, KRAS, or NRAS.

64. The compound/target protein complex or tri-complex of embodiment 63, wherein the target protein is KRAS.

65. The compound/target protein complex or tri-complex of embodiment 60, wherein the target protein is a GTPase activating factor selected from the group consisting of NF1, IQGAP1, PLEXIN-B1, RASAL1, RASAL2, ARHGAP5, ARHGAP8, ARHGAP12, ARHGAP22, ARHGAP25, BCR, DLC1, DLC2, DLC3, GRAF, RALBP1, RAP1GAP, SIPA1, TSC2, AGAP2, ASAP1, and ASAP3.

66. The compound/target protein complex or tri-complex of embodiment 60, wherein the target protein is a Guanine nucleotide-exchange factor selected from the group consisting of CNRASGEF, RASGEF1A, RASGRF2, RASGRP1, RASGRP4, SOS1, RALGDS, RGL1, RGL2, RGR, ARHGEF10, ASEF/ARHGEF4, ASEF2, DBS, ECT2, GEF-H1, LARG, NET1, OBSCURIN, P-REX1, P-REX2, PDZ-RHOGEF, TEM4, TIAM1, TRIO, VAV1, VAV2, VAV3, DOCK1, DOCK2, DOCK3, DOCK4, DOCK8, DOCK10, C3G, BIG2/ARFGEF2, EFA6, FBX8, and GEP100.

67. The compound/target protein complex of any one of embodiments 18 to 58, or the tri-complex of embodiment 59, wherein the target protein is a protein with a protein-protein interaction domain selected from the group consisting of ARM, BAR, BEACH, BH, BIR, BRCT, BROMO, BTB, C₁, C₂, CARD, CC, CALM, CH, CHROMO, CUE, DEATH, DED, DEP, DH, EF-hand, EH, ENTH, EVH1, F-box, FERM, FF, FH2, FHA, FYVE, GAT, GEL, GLUE, GRAM, GRIP, GYF, HEAT, HECT, IQ, LRR, MBT, MH1, MH2, MIU, NZF, PAS, PB1, PDZ, PH, POLO-Box, PTB, PUF, PWWP, PX, RGS, RING, SAM, SC, SH2, SH3, SOCS, SPRY, START, SWIRM, TIR, TPR, TRAF, SNARE, TUBBY, TUDOR, UBA, UEV, UIM, VHL, VHS, WD40, WW, SH2, SH3, TRAF, Bromodomain, and TPR.

68. The compound/target protein complex or tri-complex of embodiment 60, wherein the target protein is a heat shock protein selected from the group consisting of Hsp20, Hsp27, Hsp70, Hsp84, alpha B crystalline, TRAP-1, hsf1, and Hsp90.

69. The compound/target protein complex or tri-complex of embodiment 60, wherein the target protein is an ion channel selected from the group consisting of Cav2.2, Cav3.2, IKACh, Kv1.5, TRPA1, NAv1.7, Nav1.8, Nav1.9, P2X3, or P2X4.

70. The compound/target protein complex or tri-complex of embodiment 60, wherein the target protein is a coiled-coil protein selected from the group consisting of geminin, SPAG4, VAV1, MAD1, ROCK1, RNF31, NEDP1, HCCM, EEA1, Vimentin, ATF4, Nemo, SNAP25, Syntaxin 1 a, FYCO1, and CEP250.

71. The compound/target protein complex or tri-complex of embodiment 60, wherein the target protein is a kinase selected from the group consisting of ABL, ALK, AXL, BTK, EGFR, FMS, FAK, FGFR1, 2, 3, 4, FLT3, HER2/ErbB2, HER3/ErbB3, HER4/ErbB4, IGF1R, INSR, JAK1, JAK2, JAK3, KIT, MET, PDGFRA, PDGFRB, RET RON, ROR1, ROR2, ROS, SRC, SYK, TIE1, TIE2, TRKA, TRKB, KDR, AKT1, AKT2, AKT3, PDK1, PKC, RHO, ROCK1, RSK1, RKS2, RKS3, ATM, ATR, CDK1, CDK2, CDK3, CDK4, CDK5, CDK6, CDK7, CDK8, CDK9, CDK10, ERK1, ERK2, ERK3, ERK4, GSK3A, GSK3B, JNK1, JNK2, JNK3, AurA, ARuB, PLK1, PLK2, PLK3, PLK4, IKK, KIN1, cRaf, PKN3, c-Src, Fak, PyK2, and AMPK.

72. The compound/target protein complex or tri-complex of embodiment 60, wherein the target protein is a phosphatase selected from the group consisting of WIP1, SHP2, SHP1, PRL-3, PTP1B, and STEP.

73. The compound/target protein complex or tri-complex of embodiment 60, wherein the target protein is a ubiquitin ligase selected from the group consisting of BMI-1, MDM2, NEDD4-1, Beta-TRCP, SKP2, E6AP, and APC/C.

74. The compound/target protein complex or tri-complex of embodiment 60, wherein the target protein is a chromatin modifier/remodeler encoded by a gene selected from the group consisting of BRG1, BRM, ATRX, PRDM3, ASH1L, CBP, KAT6A, KAT6B, MLL, NSD1, SETD2, EP300, KAT2A, and CREBBP.

75. The compound/target protein complex or tri-complex of embodiment 60, wherein the target protein is a transcription factor encoded by a gene selected from the group consisting of EHF, ELF1, ELF3, ELF4, ELF5, ELK1, ELK3, ELK4, ERF, ERG, ETS1, ETV1, ETV2, ETV3, ETV4, ETV5, ETV6, FEV, FLI1, GAVPA, SPDEF, SP11, SPIC, SPIB, E2F1, E2F2, E2F3, E2F4, E2F7, E2F8, ARNTL, BHLHA15, BHLHB2, BHLBHB3, BHLHE22, BHLHE23, BHLHE41, CLOCK, FIGLA, HAS5, HES7, HEY1, HEY2, ID4, MAX, MESP1, MLX, MLXIPL, MNT, MSC, MYF6, NEUROD2, NEUROG2, NHLH1, OLIG1, OLIG2, OLIG3, SREBF2, TCF3, TCF4, TFAP4, TFE3, TFEB, TFEC, USF1, ARF4, ATF7, BATF3, CEBPB, CEBPD, CEBPG, CREB3, CREB3L1, DBP, HLF, JDP2, MAFF, MAFG, MAFK, NRL, NFE2, NFIL3, TEF, XBP1, PROX1, TEAD1, TEAD3, TEAD4, ONECUT3, ALX3, ALX4, ARX, BARHL2, BARX, BSX, CART1, CDX1, CDX2, DLX1, DLX2, DLX3, DLX4, DLX5, DLX6, DMBX1, DPRX, DRGX, DUXA, EMX1, EMX2, EN1, EN2, ESX1, EVX1, EVX2, GBX1, GBX2, GSC, GSC2, GSX1, GSX2, HESX1, HMX1, HMX2, HMX3, HNF1A, HNF1B, HOMEZ, HOXA1, HOXA10, HOXA13, HOXA2, HOXAB13, HOXB2, HOXB3, HOXB5, HOXC10, HOXC11, HOXC12, HOXC13, HOXD11, HOXD12, HOXD13, HOXD8, IRX2, IRX5, ISL2, ISX, LBX2, LHX2, LHX6, LHX9, LMX1A, LMX1B, MEIS1, MEIS2, MEIS3, MEOX1, MEOX2, MIXL1, MNX1, MSX1, MSX2, NKX2-3, NKX2-8, NKX3-1, NKX3-2, NKX6-1, NKX6-2, NOTO, ONECUT1, ONECUT2, OTX1, OTX2, PDX1, PHOX2A, PHOX2B, PITX1, PITX3, PKNOX1, PROP1, PRRX1, PRRX2, RAX, RAXL1, RHOXF1, SHOX, SHOX2, TGIF1, TGIF2, TGIF2LX, UNCX, VAX1, VAX2, VENTX, VSX1, VSX2, CUX1, CUX2, POU1F1, POU2F1, POU2F2, POU2F3, POU3F1, POU3F2, POU3F3, POU3F4, POU4F1, POU4F2, POU4F3, POU5F1P1, POU6F2, RFX2, RFX3, RFX4, RFX5, TFAP2A, TFAP2B, TFAP2C, GRHL1, TFCP2, NFIA, NFIB, NFIX, GCM1, GCM2, HSF1, HSF2, HSF4, HSFY2, EBF1, IRF3, IRF4, IRF5, IRF7, IRF8, IRF9, MEF2A, MEF2B, MEF2D, SRF, NRF1, CPEB1, GMEB2, MYBL1, MYBL2, SMAD3, CENPB, PAX1, PAX2, PAX9, PAX3, PAX4, PAX5, PAX6, PAX7, BCL6B, EGR1, EGR2, EGR3, EGR4, GLIS1, GLIS2, GLI2, GLIS3, HIC2, HINFP1, KLF13, KLF14, KLF16, MTF1, PRDM1, PRDM4, SCRT1, SCRT2, SNAI2, SP1, SP3, SP4, SP8, YY1, YY2, ZBED1, ZBTB7A, ZBTB7B, ZBTB7C, ZIC1, ZIC3, ZIC4, ZNF143, ZNF232, ZNF238, ZNF282, ZNF306, ZNF410, ZNF435, ZBTB49, ZNF524, ZNF713, ZNF740, ZNF75A, ZNF784, ZSCAN4, CTCF, LEF1, SOX10, SOX14, SOX15, SOX18, SOX2, SOX21, SOX4, SOX7, SOX8, SOX9, SRY, TCF7L1, FOXO3, FOXB1, FOXC1, FOXC2, FOXD2, FOXD3, FOXG1, FOXI1, FOXJ2, FOXJ3, FOXK1, FOXL1, FOXO1, FOXO4, FOXO6, FOXP3, EOMES, MGA, NFAT5, NFATC1, NFKB1, NFKB2, TP63, RUNX2, RUNX3, T, TBR1, TBX1, TBX15, TBX19, TBX2, TBX20, TBX21, TBX4, TBX5, AR, ESR1, ESRRA, ESRRB, ESRRG, HNF4A, NR2C₂, NR2E1, NR2F1, NR2F6, NR3C₁, NR3C₂, NR4A2, RARA, RARB, RARG, RORA, RXRA, RXRB, RXRG, THRA, THRB, VDR, GATA3, GATA4, GATA5, C-myc, Max, Stat3, androgen receptor, C-Jun, C-Fox, N-Myc, L-Myc, MITF, Hif-1alpha, Hif-2alpha, Bcl6, E2F1, NF-kappaB, Stat5, and ER(coact).

76. The compound/target protein complex of any one of embodiments 18 to 58, or the tri-complex of embodiment 59, wherein the target protein is selected from the group consisting of TrkA, P2Y14, mPEGS, ASK1, ALK, Bcl-2, BCL-XL, mSIN1, RORγt, IL17RA, elF4E, TLR7R, PCSK9, IgE R, CD40, CD40L, Shn-3, TNFR1, TNFR2, IL31RA, OSMR, IL12beta1,2, Tau, FASN, KCTD 6, KCTD 9, Raptor, Rictor, RALGAPA, RALGAPB, Annexin family members, BCOR, NCOR, beta catenin, AAC 11, PLD1, PLD2, Frizzled7, RaLP, MLL-1, Myb, Ezh2, RhoGD12, EGFR, CTLA4R, GCGC (coact), Adiponectin R2, GPR 81, IMPDH2, IL-4R, IL-13R, IL-1R, IL2-R, IL-6R, IL-22R, TNF-R, TLR4, Nrlp3, and OTR.

77. A presenter protein/compound complex comprising a presenter protein and a compound of any one of embodiments 12 to 17 and 33 to 58.

78. The tri-complex of any one of embodiments 59 to 76, or the presenter protein/compound complex of embodiment 77, wherein the presenter protein is a prolyl isomerase.

79. The tri-complex of any one of embodiments 59 to 76, or the presenter protein/compound complex of embodiment 77, wherein the presenter protein is a member of the FKBP family, a member of the cyclophilin family, or PIN1.

80. The tri-complex or the presenter protein/compound complex of embodiment 79, wherein the presenter protein is a member of the FKBP family selected from the group consisting of FKBP12, FKBP12.6, FKBP13, FKBP19, FKBP22, FKBP23, FKBP25, FKBP36, FKBP38, FKBP51, FKBP52, FKBP60, FKBP65, and FKBP133.

81. The tri-complex or the presenter protein/compound complex of embodiment 80, wherein the member of the FKBP family is FKBP12, FKBP12.6, FKBP25, or FKBP52.

82. The tri-complex or the presenter protein/compound complex of embodiment 79, wherein the presenter protein is a member of the cyclophilin family selected from the group consisting of PP1A, CYPB, CYPC, CYP40, CYPE, CYPD, NKTR, SRCyp, CYPH, CWC27, CYPL1, CYP60, CYPJ, PPIL4, PPIL6, RANBP2, PPWD1, PPIAL4A, PPIAL4B, PPIAL4C, PPIAL4D, and PPIAL4G.

83. The tri-complex or the presenter protein/compound complex of embodiment 82, wherein the member of the cyclophilin family is PPIAL4A, PPIAL4B, PPIAL4C, PPIAL4D, or PPIAL4G.

84. A method of modulating a target protein, the method comprising contacting the target protein with the compound of any one of embodiments 12 to 58 or the presenter protein/compound complex of any one of embodiments 77 to 83.

85. A method of inhibiting a target protein, the method comprising contacting the target protein with the compound of any one of embodiments 12 to 58 or the presenter protein/compound complex of any one of embodiments 77 to 83.

86. A method of activating a target protein, the method comprising contacting the target protein with the compound of any one of embodiments 12 to 58 or the presenter protein/compound complex of any one of embodiments 77 to 83.

87. A method of forming a tri-complex of any one of embodiments 59 to 83, the method comprising contacting a target protein with a presenter protein/compound complex of any one of embodiments 77 to 83.

88. The method of any one of embodiments 84 to 87, wherein upon contacting the target protein, the target protein forms a covalent bond to the compound or the presenter protein/compound complex.

89. The method of embodiment 88, wherein upon contacting the target protein, an aspartic acid, glutamic acid, cysteine, glutamine, asparagine, lysine, or histidine residue of the target protein forms a covalent bond to the compound or the complex.

90. The method of embodiment 89, wherein upon contacting the target protein, an aspartic acid, glutamic acid, cysteine, glutamine, or asparagine residue of the target protein forms a covalent bond to the compound or the complex

91. A method of crosslinking a compound of any one of embodiments 12 to 58 to a second moiety, the method comprising contacting the moiety with the compound under conditions sufficient to form a covalent bond between the compound of any one of embodiments 12 to 58 and the second moiety.

92. The method of embodiment 91, wherein the second moiety is a target protein.

93. A method of forming the presenter protein/compound complex of any one of embodiments 77 to 83, the method comprising contacting a presenter protein with the compound of any one of embodiments 12 to 58 under conditions sufficient to permit the formation of a complex.

94. The method of embodiment 93, wherein the complex is formed by way of noncovalent interactions.

95. A method of forming the tri-complex of any one of embodiments 59 to 83, the method comprising:
a) contacting a presenter protein with the compound of any one of embodiments 12 to 58 under conditions sufficient to permit the formation of presenter protein/compound complex; and
b) contacting the presenter protein/compound complex with a target protein under conditions that permit the formation of a tri-complex.

96. The method of embodiment 95, wherein the presenter protein/compound complex binds to the target protein with at least 5-fold greater affinity than the presenter protein or the compound of any one of embodiments 12 to 58 alone.

97. The method of embodiment 95 or 96, wherein the presenter protein or the compound of any one of embodiments 12 to 58 do not substantially bind to the target protein in the absence of forming the presenter protein/compound complex.

98. A method of treating a disease or disorder in a subject in need thereof, the method comprising administering to the subject a therapeutically effective amount of the compound of any one of embodiments 12 to 58.

99. The method of embodiment 98, wherein the subject has previously been treated with a prior therapy.

100. The method of embodiment 98 or 99, wherein the subject has developed resistance to treatment with a prior therapy.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

### Example 1. Synthesis of Compounds

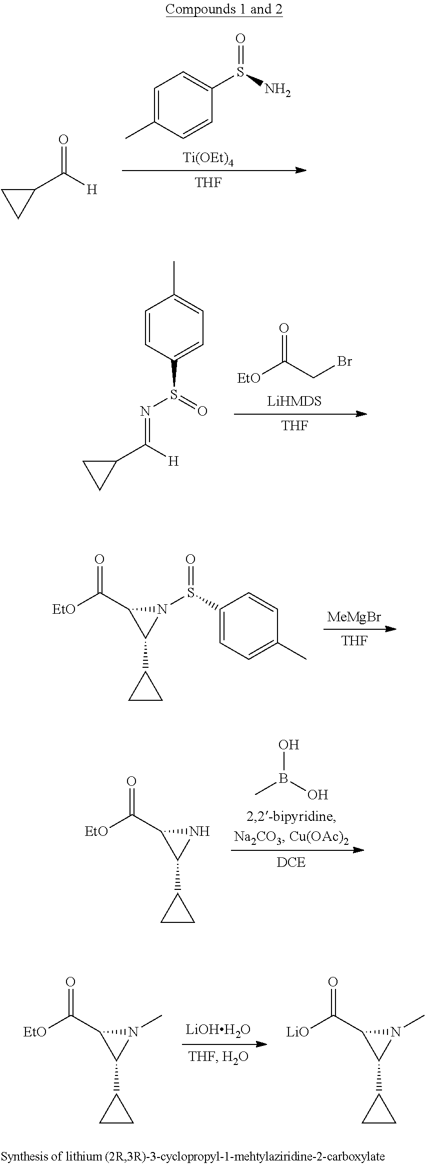

### Step 1: Synthesis of (R,E)-N-(cyclopropylmethylene)-4-methylbenzenesulfonamide

To a solution of cyclopropanecarbaldehyde (6 g, 85.60 mmol) in THF (120 mL) was added (R)-4-methylbenzenesulfonamide (13.29 g, 85.60 mmol) and Ti(OEt)₄ (39.05 g, 171.21 mmol) at room temperature under N₂. The mixture was stirred at 75° C. for 2 h. The reaction mixture was poured into brine/H₂O (1:1, 600 mL) at 0-15° C. The mixture was filtered through a pad of Celite, and the pad was washed with EtOAc (6×200 mL). The combined filtrates were extracted with EtOAc (2×200 mL). The combined organic layers were washed with brine (200 mL), dried over Na₂SO₄, filtered and concentrated under reduced pressure to give a residue. The crude product was purified by silica gel column chromatography. (0→10% EtOAc/pet. ether) to afford the product (14.6 g, 82% yield) as a solid.

### Step 2: Synthesis of ethyl (2R,3R)-3-cyclopropyl-1-((R)-p-tolylsulfinyl)aziridine-2-carboxylate

To a solution of ethyl 2-bromoacetate (23.52 g, 140.86 mmol) in THF (700 mL) was added LiHMDS (1 M, 140.86 mL) at −70° C. over 10 min under N₂. The mixture was stirred at −70° C. for 20 min. A solution of (R,E)-N-(cyclopropylmethylene)-4-methylbenzenesulfonamide (14.6 g, 70.43 mmol) in THE (150 mL) was added into the reaction solution at −70° C. over 10 min. The mixture was stirred at −70° C. for 1 h 20 min under N₂. The reaction mixture was poured into cold H₂O (1.2 L) and stirred at room temperature for 5 min. The aqueous layer was extracted with EtOAc (3×300 mL), and the combined organic layers were washed with brine (300 mL), dried over Na₂SO₄, filtered and concentrated under reduced pressure to give a residue. The crude product was purified by silica gel column chromatography. (0→10% EtOAc/pet. ether) to afford the product (11 g, 53% yield) as an oil. LCMS (ESI) m/z [M+H] calcd for C₁₅H₂₀NO₃S: 294.11; found: 294.1.

### Step 3: Synthesis of ethyl (2R,3R)-3-cyclopropylaziridine-2-carboxylate

Ethyl (2R,3R)-3-cyclopropyl-1-[(R)-p-tolylsulfinyl]aziridine-2-carboxylate (6 g, 20.45 mmol) was dissolved in anhydrous THF (300 mL). MeMgBr (3 M, 13.63 mL) was added dropwise at −65° C. over 40 min under N₂. The reaction mixture was stirred for 5 min. Sat. aq. NH₄Cl (90 mL) was added dropwise at −65° C. The cooling bath was removed, and the reaction mixture was warmed to room temperature. EtOAc (300 mL) was added, and the organic layer was separated and concentrated under reduced pressure. The residue was purified by silica gel column chromatography (0→50% EtOAC/pet. ether) to afford the product as an oil.

### Step 4: Synthesis of ethyl (2R,3R)-3-cyclopropyl-1-methylaziridine-2-carboxylate

To a solution of ethyl (2R,3R)-3-cyclopropylaziridine-2-carboxylate (400 mg, 2.58 mmol) in DCE (8 mL) was added methylboronic acid (462.85 mg, 7.73 mmol), 2,2′-bipyridine (402.54 mg, 2.58 mmol), Cu(OAc)₂ (468.14 mg, 2.58 mmol), and Na₂CO₃ (819.54 mg, 7.73 mmol). The reaction mixture was stirred at 45° C. for 40 h. The mixture was poured into aq. NH₄Cl (15 mL) and extracted with DCM (3×15 mL) and the combined organic phases were washed with brine (20 mL), dried with Na₂SO₄, filtered and concentrated under reduced pressure. The residue was purified by silica gel column chromatography (0→50% EtOAc/pet. ether) to afford the product (230 mg, 53% yield) as an oil. LCMS (ESI) m/z [M+H] calcd for C₉H₁₆NO₂: 170.1; found: 170.1.

### Step 5: Synthesis of lithium (2R,3R)-3-cyclopropyl-1-methylaziridine-2-carboxylate

To a solution of ethyl (2R,3R)-3-cyclopropyl-1-methylaziridine-2-carboxylate (230 mg, 1.36 mmol) in THF (2 mL) was added a solution of LiOH·H₂O (114.07 mg, 2.72 mmol) in H₂O (1 mL). The reaction mixture was stirred at room temperature for 1 h. The pH was adjusted to about 8 with 0.5N HCl at 0° C., and the solution was lyophilized directly to give the product (230 mg, crude) as a solid.

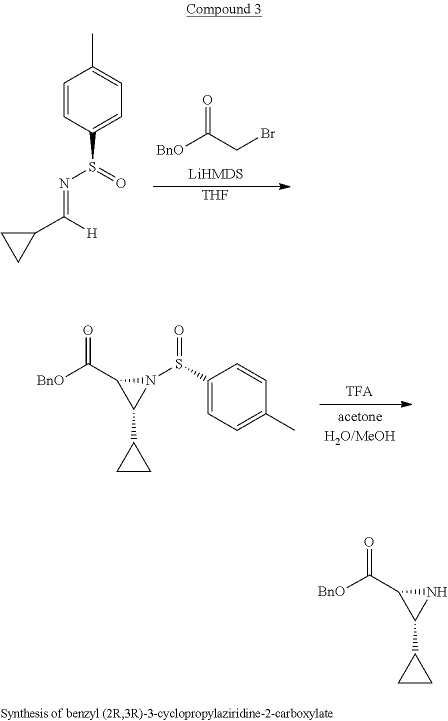

### Step 1: Synthesis of benzyl (2R,3R)-3-cyclopropyl-1-((R)-p-tolylsulfinyl)aziridine-2-carboxylate

To a solution of (R)—N-(cyclopropylmethylidene)-4-methylbenzenesulfonamide (100 g, 482.4 mmol) and benzyl 2-bromoacetate (143.66 g, 627.1 mmol) in THF (1 L) at −60° C. was added LiHMDS (627.1 mL, 627.1 mmol) dropwise over 30 min. The resulting mixture was stirred at −40° C. for 1.5 h and then cold H₂O (1.5 L) was added. The aqueous layer was extracted with EtOAc (2×1 L), and the combined organic layers were washed with H₂O (2×2 L) and brine (2 L), dried over Na₂SO₄, filtered, and concentrated under reduced pressure. The residue was purified by silica gel column chromatography (5% EtOAc/pet. ether) to afford the desired product (137 g, 80% yield) as an oil.

### Step 2: Synthesis of benzyl (2R,3R)-3-cyclopropylaziridine-2-carboxylate

To a solution of benzyl (2R,3R)-3-cyclopropyl-1-((R)-p-tolylsulfinyl)aziridine-2-carboxylate (60 g, 168.80 mmol) in acetone (786 mL), H₂O (131 mL) and MeOH (102 mL) at 0° C. was added TFA (96.24 g, 844.0 mmol). The resulting mixture was stirred at 0° C. for 60 min and then the reaction mixture was added to NH₃·H₂O (500 mL of 28% NH₃·H₂O in 1 L of H₂O) at 0° C. The resulting mixture was extracted with EtOAc (3×700 mL), and the combined organic layers were washed with H₂O (3×400 mL), dried over Na₂SO₄, filtered, and concentrated under reduced pressure. The residue was purified by silica gel column chromatography (9% EtOAc/pet. ether) to afford the desired product. LCMS (ESI) m/z [M+H] calcd for C₁₃H₁₅NO₂ 218.12; found: 218.3.

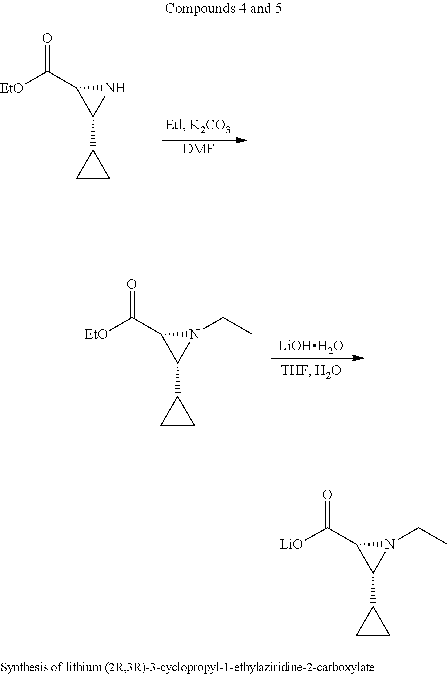

### Step 1: Synthesis of ethyl (2R,3R)-3-cyclopropyl-1-ethylaziridine-2-carboxylate

To a solution of ethyl (2R,3R)-3-cyclopropylaziridine-2-carboxylate (0.5 g, 3.22 mmol) in DMF (5 mL) at room temperature was added Etl (1.80 mL, 22.55 mmol) and K₂CO₃ (1.78 g, 12.89 mmol). The reaction mixture was stirred for 16 h and was quenched with H₂O (50 mL) at 0° C. The resulting mixture was extracted with EtOAc (3×15 mL). The combined organic layers were washed with brine (15 mL), dried over Na₂SO₄, filtered, and concentrated under reduced pressure. The residue was purified by silica gel column chromatography (0→25% EtOAc/pet. ether) to afford the product (0.35 g, 59% yield) as an oil.

### Step 2: Synthesis of lithium (2R,3R)-3-cyclopropyl-1-ethylaziridine-2-carboxylate

To a solution of ethyl (2R,3R)-3-cyclopropylaziridine-2-carboxylate (0.12 g, 654.85 μmol) in THF (1.2 mL) at 0° C. was added a solution of LiOH·H₂O (54.96 mg, 1.31 mmol) in H₂O (0.4 mL). The reaction mixture was warmed to room temperature and stirred for 4 h. The mixture was adjusted to pH=7-8 and lyophilized to afford the product (0.18 g, crude) as a white solid.

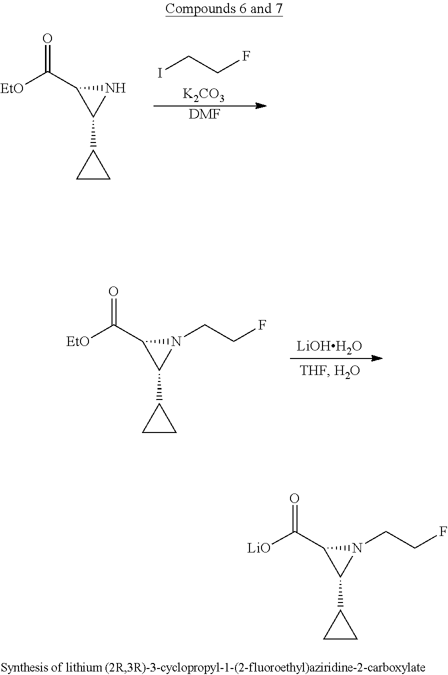

### Step 1: Synthesis of ethyl (2R,3R)-3-cyclopropyl-1-(2-fluoroethyl)aziridine-2-carboxylate

To a solution of ethyl (2R,3R)-3-cyclopropylaziridine-2-carboxylate (300 mg, 1.93 mmol) in DMF (3 mL) at room temperature was added 1-fluoro-2-iodoethane (1.68 g, 9.67 mmol) and K₂CO₃ (1.60 g, 11.60 mmol). The reaction mixture was stirred a 50° C. for 15 h. The reaction mixture was then cooled to room temperature and quenched with H₂O (30 mL). The resulting mixture was extracted with EtOAc (3×15 mL), and the combined organic layers were washed with brine, dried over Na₂SO₄, filtered, and concentrated under reduced pressure. The residue was purified by prep-TLC (33% EtOAc/pet. ether) to afford the product (120 mg, 31% yield) as an oil. LCMS (ESI) m/z [M+H] calcd for C₁₀H₁₆FNO₂: 202.12; found: 202.2.

### Step 2: Synthesis of lithium (2R,3R)-3-cyclopropyl-1-(2-fluoroethyl)aziridine-2-carboxylate

To a solution of ethyl (2R,3R)-3-cyclopropyl-1-(2-fluoroethyl)aziridine-2-carboxylate (100 mg, 496.93 μmol) in THF (1 mL) and H₂O (0.3) at room temperature was added LiOH·H₂O (41.71 mg, 993.85 μmol). The reaction mixture was stirred for 1 h. The mixture was quenched with H₂O (3 mL) and the resulting mixture was lyophilized to afford the product (90 mg, crude) as a solid.

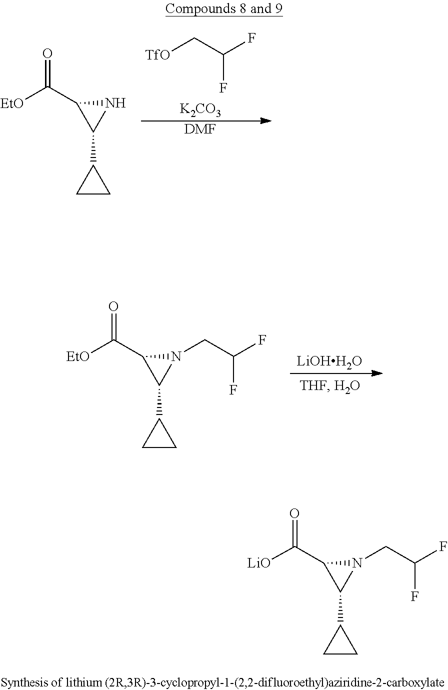

### Step 1: Synthesis of ethyl (2R,3R)-3-cyclopropyl-1-(2,2-difluoroethyl)aziridine-2-carboxylate

To a solution of ethyl (2R,3R)-3-cyclopropylaziridine-2-carboxylate (120 mg, 773.23 μmol) in DMF (2 mL) at room temperature was added 2,2-difluoroethyl trifluoromethanesulfonate (281.45 mg, 1.31 mmol) and K₂CO₃ (534.32 mg, 3.87 mmol). The reaction mixture was stirred for 15 h and was then quenched with sat. NH₄Cl (5 mL). The resulting mixture was extracted with EtOAc (3×3 mL), and the combined organic layers were washed with brine (5 mL), dried over Na₂SO₄, filtered, and concentrated under reduced pressure. The residue was purified by column chromatography (0→25% EtOAc/pet. ether) to afford the product (80 mg, 47% yield) as an oil.

### Step 2: Synthesis of lithium (2R,3R)-3-cyclopropyl-1-(2,2-difluoroethyl)aziridine-2-carboxylate

To a solution of ethyl (2R,3R)-3-cyclopropyl-1-(2,2-difluoroethyl)aziridine-2-carboxylate (130 mg, 592.99 μmol) in THF (1.3 mL) at room temperature was added a solution of LiOH·H₂O (49.77 mg, 1.19 mmol) in H₂O (0.6 mL). The reaction mixture was stirred for 1 h. The mixture was quenched with H₂O (4 mL) and the resulting mixture was lyophilized to afford the product (100 mg, crude) as a solid.

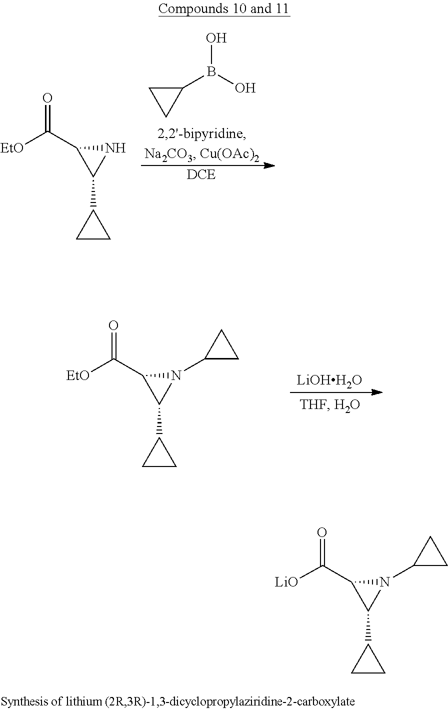

### Step 1: ethyl (2R,3R)-1,3-dicyclopropylaziridine-2-carboxylate

To a solution of ethyl (2R,3R)-3-cyclopropylaziridine-2-carboxylate (50 mg, 322.18 μmol) in DCE (1 mL) was added cyclopropylboronic acid (83.02 mg, 966.53 μmol), 2,2′-bipyridine (50.32 mg, 322.18 μmol), Cu(OAc)₂ (58.52 mg, 322.18 μmol), and Na₂CO₃ (102.44 mg, 966.53 μmol). The reaction mixture was stirred at room temperature for 16 h. The mixture was poured into brine (5 mL) and extracted with DCM (3×3 mL). The combined organic phases were washed with brine, dried with Na₂SO₄, filtered and concentrated under reduced pressure. The residue was purified by prep-TLC (33% EtOAc/pet. ether) to give the product (35 mg, 56% yield) as an oil.

### Step 2: lithium (2R,3R)-1,3-dicyclopropylaziridine-2-carboxylate

To a solution of ethyl (2R,3R)-1,3-dicyclopropylaziridine-2-carboxylate (80 mg, 409.72 μmol) in THF (1 mL) was added a solution of LiOH·H₂O (34.39 mg, 819.43 μmol) in H₂O (0.5 mL). The reaction mixture was stirred at room temperature for 1 h and was then quenched with H₂O (4 mL). The solution was lyophilized directly to give the product (100 mg, crude) as a solid.

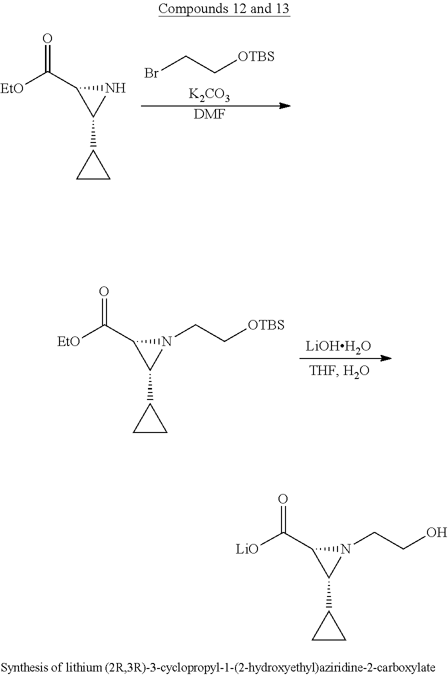

### Step 1: Synthesis of ethyl (2R,3R)-1-(2-((tert-butyldimethylsilyl)oxy)ethyl)-3-cyclopropylaziridine-2-carboxylate

To a solution of ethyl (2R,3R)-3-cyclopropylaziridine-2-carboxylate (320 mg, 2.06 mmol) in DMF (3 mL) at room temperature was added (2-bromoethoxy)(tert-butyl)dimethylsilane (2.46 g, 10.30 mmol) and K₂CO₃ (1.14 g, 8.24 mmol). The reaction mixture was heated to 75° C. and stirred for 24 h. The reaction mixture was then cooled to room temperature and quenched with H₂O (50 mL). The resulting mixture was extracted with EtOAc (3×30 mL), and the combined organic layers were washed with brine (30 mL), dried over Na₂SO₄, filtered, and concentrated under reduced pressure. The residue was purified by column chromatography (0→100% EtOAc/pet. ether) to afford the product (410 mg, 64% yield) as an oil. LCMS (ESI) m/z [M+H] calcd for C₁₆H₃₁NO₃Si: 314.22; found: 314.1.

### Step 2: Synthesis of lithium (2R,3R)-3-cyclopropyl-1-(2-hydroxyethyl)aziridine-2-carboxylate

To a solution of ethyl (2R,3R)-1-(2-((tert-butyldimethylsilyl)oxy)ethyl)-3-cyclopropylaziridine-2-carboxylate (410 mg, 1.31 mmol) in THF (4 mL) at 0° C. was added a solution of LiOH·H₂O (219.50 mg, 5.23 mmol) in H₂O (2 mL). The reaction mixture was stirred for 24 h. The mixture was quenched with H₂O (5 mL) and the resulting mixture was lyophilized to afford the product (330 mg, crude) as a solid. LCMS (ESI) m/z [M+H] calcd for C₈H₁₃NO₃: 172.09; found: 172.1.

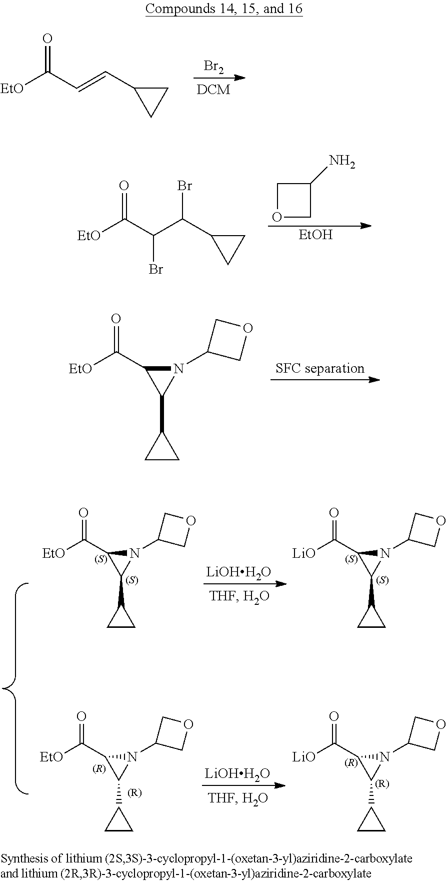

### Step 1: Synthesis of ethyl 2,3-dibromo-3-cyclopropylpropanoate

To a solution of Br₂ (5.0 mL, 97.38 mmol) in DCM (30 mL) was added to a solution of ethyl (E)-3-cyclopropylacrylate (13 g, 92.74 mmol) in DCM (100 mL). The mixture was stirred at room temperature for 5 min. The reaction mixture was then added to H₂O (100 mL) and the aqueous layer was extracted with DCM (3×50 mL). The combined organic layers were washed with brine (80 mL), dried with Na₂SO₄, filtered, and concentrated under reduced pressure. The residue was purified by silica gel column chromatography (0→9% EtOAc/pet. ether) to afford the desired product (8 g, 29% yield) as an oil.

### Step 2: Synthesis of ethyl 3-cyclopropyl-1-(oxetan-3-yl)aziridine-2-carboxylate

To a solution of oxetan-3-amine (65.79 g, 900.0 mmol) in EtOH (45 mL) at 0° C. was added a solution of ethyl 2,3-dibromo-3-cyclopropylpropanoate (9 g, 30.0 mmol) in EtOH (45 mL). The reaction mixture was warmed to room temperature and stirred for 16 h. The reaction mixture was concentrated under reduced pressure. The crude product was purified by silica gel chromatography (0→50% EtOAc/pet. ether) to afford the desired product (3.9 g, 62% yield) as a solid. LCMS (ESI) m/z [M+H] calcd for C₁₁H₁₇NO₃: 212.13; found: 212.1.

### Step 3: Synthesis of ethyl (2S,3S)-3-cyclopropyl-1-(oxetan-3-yl)aziridine-2-carboxylate and ethyl

(2R,3R)-3-cyclopropyl-1-(oxetan-3-yl)aziridine-2-carboxylate Ethyl 3-cyclopropyl-1-(oxetan-3-yl)aziridine-2-carboxylate (1.5 g) was purified by prep-SFC (Daicel CHIRALPAK IC (250 mm×30 mm, 10 μm); 25% EtOH/CO₂) then the solution was concentrated under reduced pressure to afford ethyl (2S,3S)-3-cyclopropyl-1-(oxetan-3-yl)aziridine-2-carboxylate (600 mg, 40% yield) and ethyl (2R,3R)-3-cyclopropyl-1-(oxetan-3-yl)aziridine-2-carboxylate (600 mg, 40% yield) as oils. LCMS (ESI) m/z [M+H] calcd for C₁₁H₁₇NO₃: 212.13; found: 212.1.

### Step 4: Synthesis of lithium (2S,3S)-3-cyclopropyl-1-(oxetan-3-yl)aziridine-2-carboxylate

To a solution of ethyl (2S,3S)-3-cyclopropyl-1-(oxetan-3-yl)aziridine-2-carboxylate (250 mg, 1.18 mmol) in THF (2.5 mL) at 0° C. was added a solution of LiOH·H₂O (99.31 mg, 2.37 mmol) in H₂O (1.25 mL). The reaction mixture was warmed to room temperature and stirred for 1 h. The mixture was diluted with H₂O (4 mL) and lyophilized directly to afford the desired product (200 mg, 89% yield) as a solid.

### Step 5: Synthesis of lithium (2R,3R)-3-cyclopropyl-1-(oxetan-3-yl)aziridine-2-carboxylate

To a solution of ethyl (2S,3S)-3-cyclopropyl-1-(oxetan-3-yl)aziridine-2-carboxylate (250 mg, 1.18 mmol) in THF (2.5 mL) at 0° C. was added a solution of LiOH·H₂O (99.32 mg, 2.37 mmol) in H₂O (1.25 mL). The reaction mixture was warmed to room temperature and stirred for 1 h. The mixture was diluted with H₂O (4 mL) and lyophilized directly to afford the desired product (200 mg, 89% yield) as a solid.

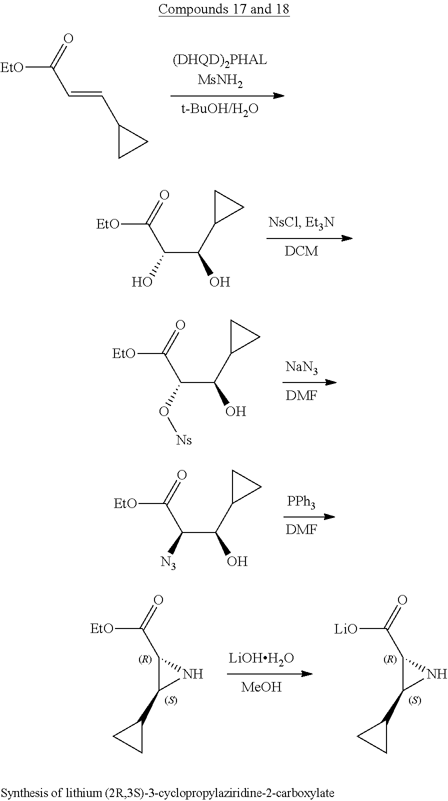

### Step 1: Synthesis of ethyl (2S,3R)-3-cyclopropyl-2,3-dihydroxypropanoate

A solution of ethyl (E)-3-cyclopropylacrylate (10.4 mL, 71 mmol) in tert-BuOH (270 mL) and H₂O (270 mL) was stirred at 0° C. After 5 min MsNH₂ (6.8 g, 71 mmol) and (DHQD)₂PHAL (100 g, 130 mmol) were added and the reaction mixture was warmed to room temperature. After stirring overnight, sat. Na₂SO₃ was added and the mixture was stirred for 30 min. The mixture was acidified to pH 6 with KH₂PO₄. Purification by silica gel column chromatography (33% EtOAc/pet. ether) afforded desired product (5.5 g, 44% yield).

### Step 2: Synthesis of ethyl (2S,3R)-3-cyclopropyl-3-hydroxy-2-(((4-nitrophenyl)sulfonyl)oxy)propanoate

A solution of ethyl (2S,3R)-3-cyclopropyl-2,3-dihydroxypropanoate (5.40 g, 31.0 mmol) and Et₃N (13.0 mL, 93.0 mmol) in DCM (20 mL) was stirred at 0° C. and a solution of 4-nitrobenzenesulfonyl chloride (6.53 g, 29.5 mmol) in DCM (10 mL) was added. The reaction mixture was stirred for 1.5 h and was then extracted with DCM (3×200 mL). The combined organic layers were washed with brine (100 mL), dried with Na₂SO₄, filtered, and concentrated under reduced pressure. Purification by silica gel column chromatography (33% EtOAc/pet. ether) afforded desired product (6.9 g, 62% yield).

### Step 3: Synthesis of ethyl (2R,3R)-2-azido-3-cyclopropyl-3-hydroxypropanoate

A mixture of ethyl (2S,3R)-3-cyclopropyl-3-hydroxy-2-(((4-nitrophenyl)sulfonyl)oxy)propanoate (6.90 g, 19.2 mmol) and NaN₃ (6.24 g, 96.0 mmol) in DMF (70.0 mL) was heated to 50° C. The reaction mixture was stirred for 5 h and then extracted with EtOAc (3×200 mL). The combined organic layers were washed with brine (100 mL), dried with Na₂SO₄, filtered, and concentrated under reduced pressure. Purification by silica gel column chromatography (20% EtOAc/pet. ether) afforded desired product (2.8 g, 73% yield).

### Step 4: Synthesis of ethyl (2R,3S)-3-cyclopropylaziridine-2-carboxylate

A mixture of triphenylphosphine (1.84 g, 7.02 mmol) in DMF (5 mL) was stirred at 0° C. After 5 min ethyl (2R,3R)-2-azido-3-cyclopropyl-3-hydroxypropanoate (1.40 g, 7.03 mmol) was added and the reaction was warmed to room temperature. The reaction mixture was heated to 80° C. and stirred for 1 h. The mixture was then cooled to room temperature and extracted with EtOAc (3×50 mL). The combined organic layers were washed with brine (50 mL), dried with Na₂SO₄, filtered, and concentrated under reduced pressure. Purification by silica gel column chromatography (20% EtOAc/pet. ether) afforded the desired product (230 mg, 46% yield). LCMS (ESI) m/z: [M+H] calcd for C₈H₁₃NO₂: 156.10; found 156.2.

### Step 5: Synthesis of lithium (2R,3S)-3-cyclopropylaziridine-2-carboxylate

To a mixture of ethyl (2R,3S)-3-cyclopropylaziridine-2-carboxylate (230 mg, 1.5 mmol) in MeOH (3.0 mL) was added LiOH·H₂O (125 mg, 3.0 mmol). The reaction was stirred for 3 h and then filtered. The filtrate was concentrated under reduced pressure which afforded the desired product (150 mg, crude). LCMS (ESI) m/z: [M+H] calcd for C₆H₉NO₂: 128.07; found 128.2.

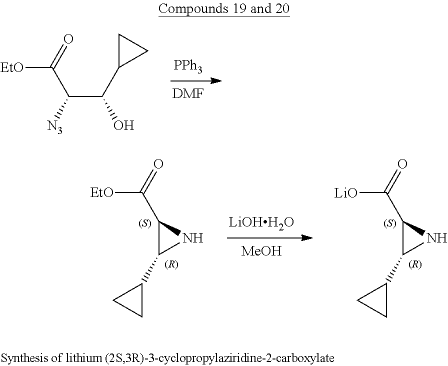

### Step 1: Synthesis of ethyl (2S,3R)-3-cyclopropylaziridine-2-carboxylate

A mixture of PPh₃ (1.4 g, 5.4 mmol) in DMF (15.0 mL) was stirred at 0° C. After 30 min, ethyl (2S,3S)-2-azido-3-cyclopropyl-3-hydroxypropanoate (980 mg, 4.92 mmol) was added. The reaction mixture was heated to 80° C. After 2 h the reaction was quenched by the addition of H₂O (20 mL) and was extracted with EtOAc (3×30 mL). Purification by silica gel column chromatography (17% EtOAc/pet. ether) afforded desired product (500 mg, 65% yield).

### Step 2: Synthesis of lithium (2S,3R)-3-cyclopropylaziridine-2-carboxylate

To a solution of ethyl (2S,3R)-3-cyclopropylaziridine-2-carboxylate (450 mg, 2.9 mmol) in THE (6.0 mL) and H₂O (2.0 mL) was added LiOH (90 mg, 3.8 mmol). The reaction was stirred for 2 h and then filtered. The filtrate was concentrated under reduced pressure which afforded the desired product (300 mg, crude).

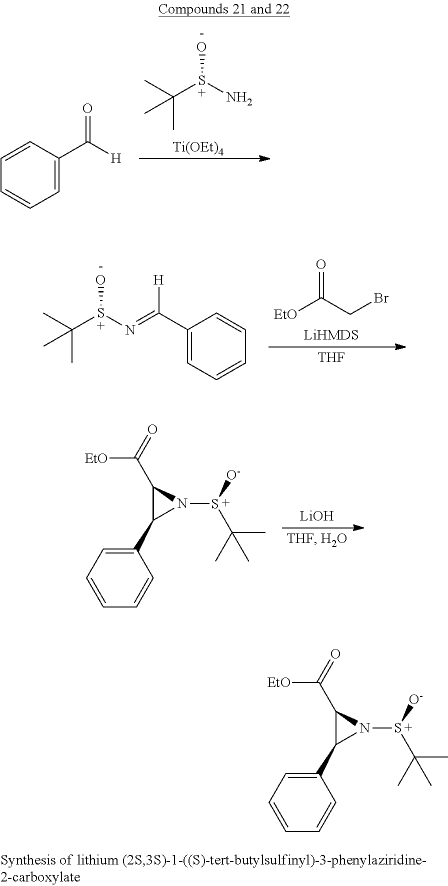

### Step 1: Synthesis of (S,E)-N-benzylidene-2-methylpropane-2-sulfinamide

A solution of (S)-2-methylpropane-2-sulfinamide (2.50 g, 20.6 mmol), titanium ethoxide (9.41 g, 41.25 mmol) and benzaldehyde (2.19 g, 20.7 mmol) was heated at 70° C. for 1 h, cooled, and diluted with H₂O (250 mL). The aqueous layer was extracted with EtOAc (3×80 mL), and the combined organic layers were washed with brine (2×100 mL), dried with Na₂SO₄, filtered and concentrated under reduced pressure to afford the desired product (4.3 g, crude) which was used without further purification. LCMS (ESI) m/z: [M+H] calcd for C₁₁H₁₅NOS: 210.10; found 210.2.

### Step 2: Synthesis of ethyl (2S,3S)-1-((S)-tert-butylsulfinyl)-3-phenylaziridine-2-carboxylate

To a solution of ethyl bromoacetate (798 mg, 4.78 mmol) in THF (15 mL) at −78° C. was added LiHMDS (1 M in THF, 4.78 mL, 4.78 mmol). After 1 h, (S,E)-N-benzylidene-2-methylpropane-2-sulfinamide (500 mg, 2.39 mmol) in THF (5 mL) was added in portions over 20 min. The reaction mixture was stirred at −78° C. for 2 h and then quenched by the addition of sat. NH₄Cl. The aqueous layer was extracted with EtOAc (3×40 mL), and the combined organic layers were washed with brine (2×30 mL), dried with Na₂SO₄, filtered, and concentrated under reduced pressure. Purification by reverse phase chromatography (30→60% MeCN/H₂O, 0.1% HCO₂H) afforded the desired product (480 mg, 61% yield). LCMS (ESI) m/z: [M+H] calcd for C₁₅H₂₁NO₃S: 296.13; found 296.2.

### Step 3: Synthesis lithium (2S,3S)-1-((S)-tert-butylsulfinyl)-3-phenylaziridine-2-carboxylate

To a solution of ethyl (2S,3S)-1-((S)-tert-butylsulfinyl)-3-phenylaziridine-2-carboxylate (600 mg, 2.03 mmol) in THF (4.0 mL) at 0° C. was added a solution of LiOH (97.2 mg, 4.06 mmol) in H₂O (4.0 mL). The resulting mixture was stirred for 2 h at 0° C. and then acidified to pH 5 with 1 M HCl. The aqueous layer was extracted with EtOAc (3×40 mL), and the combined organic layers were washed with brine (2×20 mL), dried with Na₂SO₄, filtered, and concentrated under reduced pressure to afford the desired compound (450 mg, crude) which was used without further purification. LCMS (ESI) m/z: [M+H] calcd for C₁₃H₁₇NO₃S: 268.10; found 268.1.

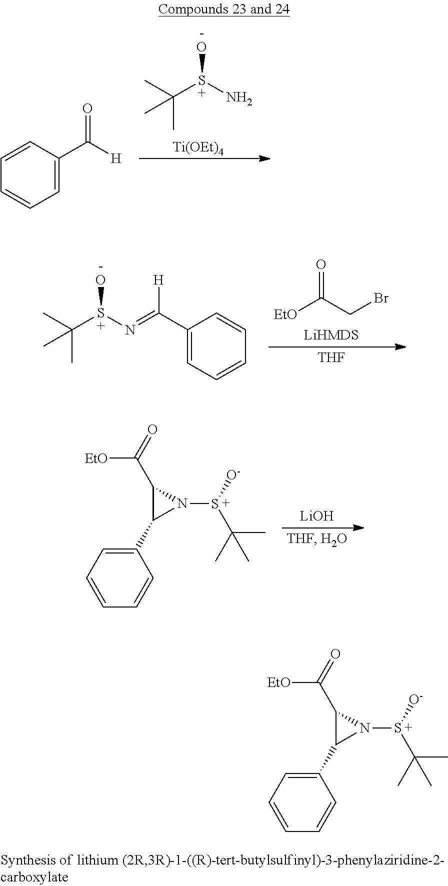

### Step 1: Synthesis (R,E)-N-benzylidene-2-methylpropane-2-sulfinamide

A solution (R)-2-methylpropane-2-sulfinamide (2.50 g, 20.6 mmol), titanium tetraethoxide (9.41 g, 41.3 mmol) and benzaldehyde (2.19 g, 20.6 mmol) was heated 70° C. for 1 h, cooled, and diluted with H₂O (250 mL). The aqueous layer was extracted with EtOAc (3×90 mL), and the combined organic layers were washed with brine (2×100 mL), dried with Na₂SO₄, filtered and concentrated under reduced pressure to afford the desired product (4.2 g, crude) which was used without further purification. LCMS (ESI) m/z: [M+H] calcd for C₁₁H₁₅NOS: 210.10; found 210.1.

### Step 2: Synthesis of ethyl (2R,3R)-1-((R)-tert-butylsulfinyl)-3-phenylaziridine-2-carboxylate

To a solution of ethyl bromoacetate (6.38 g, 38.2 mmol) in THF (150 mL) at −78° C. was added LiHMDS (1 M in THF, 7.19 mL, 42.9 mmol). After 1 h, (R,E)-N-benzylidene-2-methylpropane-2-sulfinamide (4.0 g, 19.1 mmol) in THF (50 mL) was added in portions over 20 min. The reaction mixture was stirred at −78° C. for 2 h and then quenched by the addition of sat. NH₄Cl. The aqueous layer was extracted with EtOAc (3×80 mL), and the combined organic layers were washed with brine (2×60 mL), dried with Na₂SO₄, filtered and concentrated under reduced pressure. Purification by reverse phase chromatography (30→60% MeCN/H₂O, 0.1% HCO₂H) afforded the desired product (3.9 g, 62% yield). LCMS (ESI) m/z: [M+H] calcd for C₁₅H₂₁NO₃S: 296.13; found 296.2.

### Step 3: Synthesis lithium (2R,3R)-1-((R)-tert-butylsulfinyl)-3-phenylaziridine-2-carboxylate

To a solution of ethyl (2R,3R)-1-((R)-tert-butylsulfinyl)-3-phenylaziridine-2-carboxylate (200 mg, 0.677 mmol) in THF (1.5 mL) at 0° C. was added a solution of LiOH (32.4 mg, 1.35 mmol) in H₂O (1.3 mL). The resulting mixture was stirred for 2 h at 0° C. and then acidified to pH 5 with 1 M HCl. The aqueous layer was extracted with EtOAc (3×20 mL), and the combined organic layers were washed with brine (2×10 mL), dried with Na₂SO₄, filtered, and concentrated under reduced pressure to afford the desired compound (220 mg, crude) which was used without further purification. LCMS (ESI) m/z: [M+H] calcd for C₁₃H₁₇NO₃S: 268.10; found 268.4.

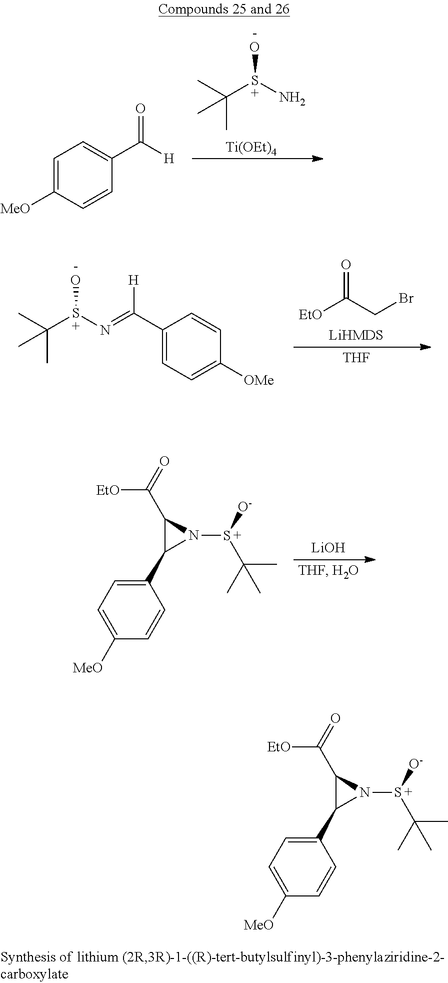

### Step 1: Synthesis of (E)-N-(4-methoxybenzylidene)-2-methylpropane-2-sulfinamide

A solution of (S)-2-methylpropane-2-sulfinamide (2.50 g) and anisaldehyde (2.81 g) in Ti(OEt)₄ (20.0 mL) was stirred at 70° C. for 1 h. The resulting mixture was cooled to room temperature, diluted with EtOAc (60 mL), and then poured into H₂O. The mixture was filtered, and the filter cake was washed with EtOAc (3×50 mL). The resulting mixture was extracted with EtOAc (3×50 mL), and the combined organic layers were washed with brine (50 mL), dried over Na₂SO₄, filtered, and concentrated under reduced pressure. The residue was purified by silica gel column chromatography (25% EtOAc/pet. ether) to afford the desired product (4 g, 81% yield). LCMS (ESI) m/z: [M+H] calcd for C₁₂H₁₇NO₂S: 240.11; found 240.1.

### Step 2: Synthesis of ethyl (2S,3S)-1-(tert-butylsulfinyl)-3-(4-methoxyphenyl)aziridine-2-carboxylate

To a solution of ethyl 2-bromoacetate (5.60 g, 33.5 mmol) in THF (100 mL) at −78° C. was added LiHMDS (1 M in THF, 34 mL, 33.473 mmol). After 30 min a solution of (E)-N-(4-methoxybenzylidene)-2-methylpropane-2-sulfinamide (4 g, 16.74 mmol) in THF (20 mL) was added. The resulting mixture was stirred at −78° C. for an additional 3 h. The reaction was then quenched with sat. aq. NH₄Cl. The mixture was extracted with EtOAc (3×100 mL), and the combined organic layers were washed with brine (50 mL), dried over Na₂SO₄, filtered, and concentrated under reduced pressure. The residue was purified by silica gel chromatography (25% EtOAc/pet. Ether) to afford the desired product (2.7 g, 50% yield). LCMS (ESI) m/z: [M+H] calcd for C₁₆H₂₃NO₄S: 326.14; found 326.1.

### Step 3: Synthesis of lithium (2S,3S)-1-((S)-tert-butylsulfinyl)-3-(4-methoxyphenyl)aziridine-2-carboxylate

To a solution of ethyl (2S,3S)-1-(tert-butylsulfinyl)-3-(4-methoxyphenyl)aziridine-2-carboxylate (800.0 mg, 2.68 mmol) in THF (2.0 mL) at 0° C. was added a solution of LiOH·H₂O (309.46 mg, 7.38 mmol) in H₂O (3.0 mL). The resulting mixture was warmed to room temperature and stirred for 4 h. The mixture was then acidified to pH 6 with sat. aq. NH₄Cl and then extracted with EtOAc (3×50 mL). The combined organic layers were dried over Na₂SO₄, filtered. And concentrated under reduced pressure to afford the desired product (690 mg, 94% yield). LCMS (ESI) m/z: [M−H] calcd for C₁₄H₁₉NO₄S: 296.10; found 296.2.

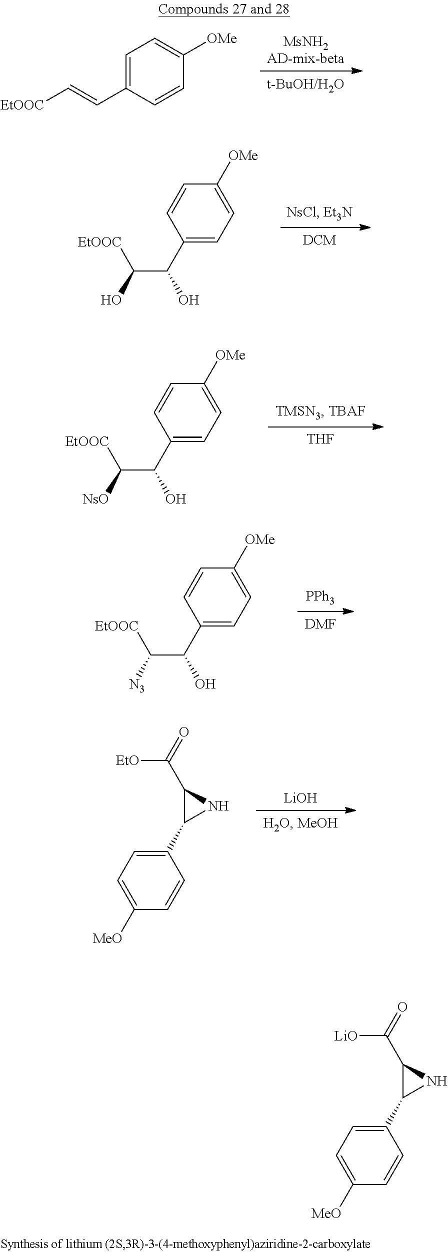

### Step 1: Synthesis of ethyl (2R,3S)-2,3-dihydroxy-3-(4-methoxyphenyl)propanoate

To a solution of ethyl p-methoxycinnamate (5.0 g, 24.24 mmol) in tBuOH (70.0 mL) and H₂O (70.0 mL) at 0° C. was added AD-mix-α (33.80 g, 43.39 mmol) and methanesulfonamide (2.31 mg, 0.024 mmol). The resulting mixture was warmed to room temperature and stirred overnight. The reaction was then cooled to 0° C. and quenched with KHSO₄ (aq.). The mixture was extracted with EtOAc (3×100 mL), and the combined organic layers were washed with brine (2×90 mL), dried over Na₂SO₄, filtered, and concentrated under reduced pressure. The residue was purified by silica gel chromatography (50% EtOAc/pet. ether) to afford the desired product (5.7 g, 88% yield).

### Step 2: Synthesis of ethyl (2R,3S)-3-hydroxy-3-(4-methoxyphenyl)-2-(((4-nitrophenyl)sulfonyl)oxy)propanoate

To a solution of ethyl (2R,3S)-2,3-dihydroxy-3-(4-methoxyphenyl)propanoate (3.0 g, 12.49 mmol) and Et₃N (0.174 mL, 1.249 mmol) in DCM (30.0 mL) at 0° C. was added 4-nitrobenzenesulfonyl chloride (2.76 g, 12.49 mmol). The resulting mixture was stirred for 1 h and was then diluted with H₂O. The mixture was extracted with DCM (3×100 mL) and the combined organic layers were washed with brine (2×100 mL), dried over Na₂SO₄, filtered, and concentrated under reduced pressure. The residue was purified by prep-TLC (50% EtOAc/pet. ether) to afford the desired product (3.8 g, 68% yield). LCMS (ESI) m/z: [M+Na] calcd for C₁₈H₁₉NO₉S: 448.07; found 448.2.

### Step 3: Synthesis of ethyl (2S,3S)-2-azido-3-hydroxy-3-(4-methoxyphenyl)propanoate

To a solution of ethyl (2R,3S)-3-hydroxy-3-(4-methoxyphenyl)-2-(((4-nitrophenyl)sulfonyl)oxy)propanoate (1.20 g, 2.82 mmol) in THF at 0° C. was added TBAF (1 M in THF, 5.64 mL, 5.64 mmol) and TMSN₃ (648.79 mg, 5.64 mmol). The resulting mixture was heated at 60° C. and stirred for 16 h. The reaction was then cooled to 0° C. and quenched with sat. aq. NH₄Cl. The mixture was extracted with EtOAc (3×100 mL), and the combined organic layers were washed with H₂O (2×100 mL), dried over Na₂SO₄, filtered, and concentrated under reduced pressure. The residue was purified by prep-TLC (33% EtOAc/pet. ether) to afford the desired product (540 mg, 71% yield).

### Step 4: Synthesis of ethyl (2S,3R)-3-(4-methoxyphenyl)aziridine-2-carboxylate

To a solution of ethyl (2S,3S)-2-azido-3-hydroxy-3-(4-methoxyphenyl)propanoate (440.0 mg, 1.659 mmol) in DMF was added PPh₃ (522.06 mg, 1.99 mmol). The resulting mixture was stirred at room temperature for 30 min and was then heated to 80° C. and stirred overnight. The mixture was then extracted with EtOAc (3×100 mL), and the combined organic layers were washed with H₂O (2×100 mL), dried over anhydrous Na₂SO₄, filtered, and concentrated under reduced pressure. The residue was purified by prep-TLC (25% EtOAc/pet. ether) to afford the desired product (200 mg, 52% yield). LCMS (ESI) m/z: [M+H] calcd for C₁₂H₁₅NO₃: 222.12; found 222.1.

### Step 5: Synthesis of lithium (2S,3R)-3-(4-methoxyphenyl)aziridine-2-carboxylate

To a solution of ethyl (2S,3R)-3-(4-methoxyphenyl)aziridine-2-carboxylate (200.0 mg, 0.904 mmol) in MeOH and H₂O at 0° C. was added LiOH·H₂O (86.6 mg, 3.62 mmol). The resulting mixture was stirred for 1 h and was then neutralized to pH 7 with HCl (aq.). The mixture was extracted with EtOAc (3×100 mL), and the combined organic layers were washed with H₂O (2×100 mL), dried over Na₂SO₄, filtered, and concentrated under reduced pressure to afford the desired product (180 mg, 98% yield). LCMS (ESI) m/z: [M−H] calcd for C₁₀H₁₁NO₃: 192.07; found 192.0.

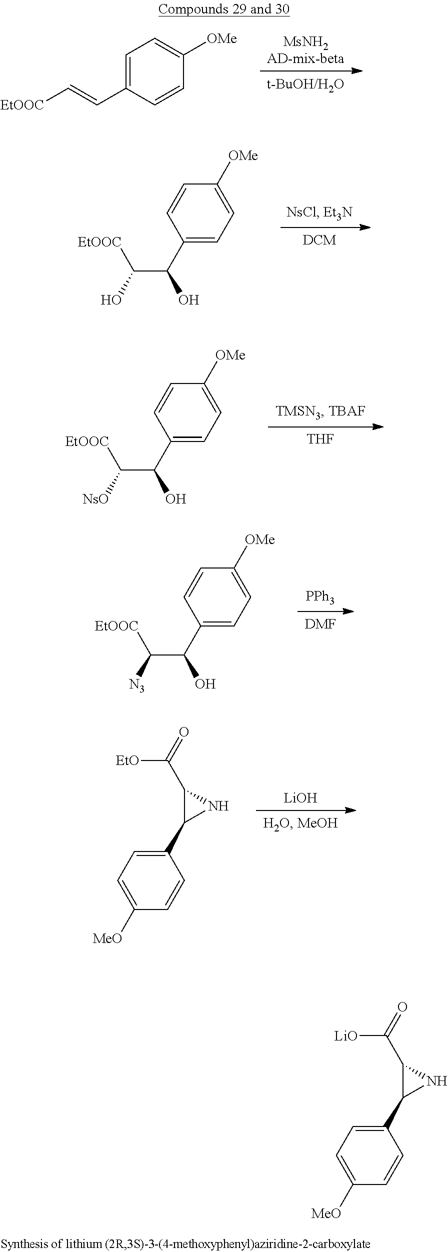

### Step 1: Synthesis of ethyl (2S,3R)-2,3-dihydroxy-3-(4-methoxyphenyl)propanoate

To a solution of ethyl p-methoxycinnamate (5.0 g, 24.24 mmol) in tBuOH (70.0 mL) and H₂O (70.0 mL) at 0° C. was added AD-mix-B (33.80 g, 43.39 mmol) and methanesulfonamide (2.31 mg, 0.024 mmol). The resulting mixture was warmed to room temperature and stirred overnight. The reaction was then cooled to 0° C. and quenched with KHSO₄ (aq.). The mixture was extracted with EtOAc (3×100 mL), and the combined organic layers were washed with brine (2×90 mL), dried over Na₂SO₄, filtered, and concentrated under reduced pressure. The residue was purified by silica gel chromatography (50% EtOAc/pet. ether) to afford the desired product (5.7 g, 88% yield).

### Step 2: Synthesis of ethyl (2S,3R)-3-hydroxy-3-(4-methoxyphenyl)-2-(((4-nitrophenyl)sulfonyl)oxy)propanoate

To a solution of ethyl (2S,3R)-2,3-dihydroxy-3-(4-methoxyphenyl)propanoate (5.80 g, 24.14 mmol) and Et₃N (10.1 mL, 72.42 mmol) in DCM (30.0 mL) at 0° C. was added 4-nitrobenzenesulfonyl chloride (5.34 g, 24.1 mmol). The resulting mixture was stirred for 1 h and was then diluted with H₂O. The mixture was extracted with DCM (3×100 mL) and the combined organic layers were washed with brine (2×100 mL), dried over Na₂SO₄, filtered, and concentrated under reduced pressure. The residue was purified by prep-TLC (50% EtOAc/pet. ether) to afford the desired product (7.2 g, 67% yield). LCMS (ESI) m/z: [M+H] calcd for C₁₈H₁₉NO₉S: 426.09; found 426.2.

### Step 3: Synthesis of ethyl (2R,3R)-2-azido-3-hydroxy-3-(4-methoxyphenyl)propanoate

To a solution of ethyl (2S,3R)-3-hydroxy-3-(4-methoxyphenyl)-2-(((4-nitrophenyl)sulfonyl)oxy)propanoate (5.0 g, 11.75 mmol) in THF at 0° C. was added TBAF (1 M in THF, 23.5 mL, 23.51 mmol) and TMSN₃ (2.7 g, 23.5 mmol). The resulting mixture was heated to 60° C. and stirred for 16 h. The reaction was then cooled to 0° C. and quenched with sat. aq. NH₄Cl. The mixture was extracted with EtOAc (3×100 mL), and the combined organic layers were washed with H₂O (2×100 mL), dried over Na₂SO₄, filtered, and concentrated under reduced pressure. The residue was purified by prep-TLC (33% EtOAc/pet. ether) to afford the desired product (2.3 g, 70% yield).

### Step 4: Synthesis of ethyl (2R,3S)-3-(4-methoxyphenyl)aziridine-2-carboxylate

To a solution of ethyl (2R,3R)-2-azido-3-hydroxy-3-(4-methoxyphenyl)propanoate (2.30 g, 8.67 mmol) in DMF was added PPh₃ (2.73 g, 10.4 mmol). The resulting mixture was stirred at room temperature for 30 min and was then heated to 80° C. and stirred overnight. The mixture was then extracted with EtOAc (3×100 mL), and the combined organic layers were washed with H₂O (2×100 mL), dried over anhydrous Na₂SO₄, filtered, and concentrated under reduced pressure. The residue was purified by prep-TLC (25% EtOAc/pet. ether) to afford the desired product (1.6 g, 79% yield). LCMS (ESI) m/z: [M+H] calcd for C₁₂H₁₅NO₃: 222.12; found 222.1.

### Step 5: Synthesis of lithium (2R,3S)-3-(4-methoxyphenyl)aziridine-2-carboxylate

To a solution of ethyl (2S,3R)-3-(4-methoxyphenyl)aziridine-2-carboxylate (200.0 mg, 0.904 mmol) in MeOH and H₂O at 0° C. was added LiOH·H₂O (86.6 mg, 3.62 mmol). The resulting mixture was stirred for 1 h and was then neutralized to pH 7 with HCl (aq.). The mixture was extracted with EtOAc (3×100 mL) and the combined organic layers were washed with H₂O (2×100 mL), dried over Na₂SO₄, filtered, and concentrated under reduced pressure to afford the desired product (180 mg, 98% yield). LCMS (ESI) m/z: [M−H] calcd for C₁₀H₁₁NO₃: 192.07; found 192.0.

### Other Embodiments

While the disclosure has been described in connection with specific embodiments thereof, it will be understood that it is capable of further modifications and this application is intended to cover any variations, uses, or adaptations of the disclosure following, in general, the principles of the disclosure and including such departures from the disclosure that come within known or customary practice within the art to which the disclosure pertains and may be applied to the essential features hereinbefore set forth, and follows in the scope of the claims. Other embodiments are within the claims.

All publications, patents and patent applications are herein incorporated by reference in their entirety to the same extent as if each individual publication, patent or patent application was specifically and individually indicated to be incorporated by reference in its entirety.

1. A compound having the structure of Formula I: 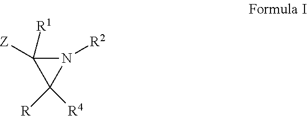
or a pharmaceutically acceptable salt thereof; wherein
M⁺ is a cation; 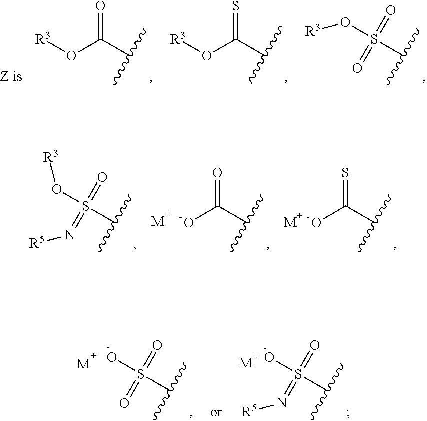
R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R and R¹ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R and R² combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; and R¹ and R⁴ are each, independently, hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a}); or
R and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R² combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and each of R and R⁴ is, independently, hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R² and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and each of R¹ and R is, independently, hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
wherein at least one of R and R⁴ is not hydrogen;
R³ is hydrogen, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₃-C₁₀ cycloalkyl, optionally substituted C₃-C₁₀ cycloalkenyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl;
each R^{1a}, R^{2a}, R^{2c}, R^{2d}, and R^{2e} is, independently, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl;
R^{2b} is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁵ is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl,
and wherein each hydrogen is independently, optionally, isotopically enriched for deuterium.

2. The compound of claim 1, or a pharmaceutically acceptable salt thereof, wherein R³ is optionally substituted C₁-C₆ alkyl, optionally substituted 3- to 10-membered heterocycloalkyl, optionally substituted C₆-C₁₀ aryl, or optionally substituted 5- to 10-membered heteroaryl.

3. The compound of claim 1 or 2, or a pharmaceutically acceptable salt thereof, wherein R³ is optionally substituted C₁-C₆ alkyl.

4. The compound of any one of claims 1 to 3, or a pharmaceutically acceptable salt thereof, wherein R³ is methyl, ethyl, or benzyl.

5. The compound of any one of claims 1 to 4, or a pharmaceutically acceptable salt thereof, wherein M⁺ is Li⁺.

6. The compound of any one of claims 1 to 5, or a pharmaceutically acceptable salt thereof, wherein R³ is:

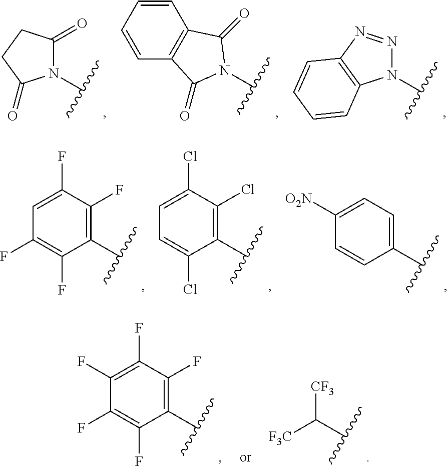

7. The compound of any one of claims 1 to 6, or a pharmaceutically acceptable salt thereof, wherein Z is

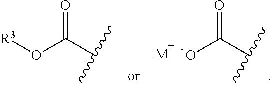

8. A compound having the structure of Formula II: 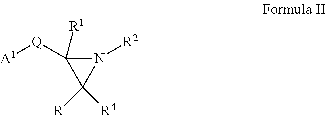
or a pharmaceutically acceptable salt thereof,
wherein A¹ is a monovalent organic moiety;
Q is 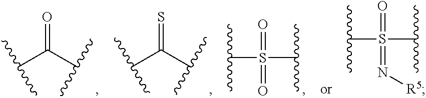
R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
or R and R¹ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R and R² combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and R¹ and R⁴ are each, independently, hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a}); or
R and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl; R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R² combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and each of R and R⁴ is, independently, hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R² and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and each of R¹ and R is, independently, hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
wherein at least one of R and R⁴ is not hydrogen;
each R^{1a}, R^{2a}, R^{2c}, R^{2d}, and R^{2e} is, independently, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl;
R^{2b} is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁵ is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl,
and wherein each hydrogen is independently, optionally, isotopically enriched for deuterium.

9. The compound of claim 8, or a pharmaceutically acceptable salt thereof, wherein Q is

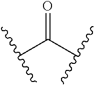 [SMILES: *C(*)=O]

10. A compound/target protein complex, or a pharmaceutically acceptable salt thereof, wherein the compound/target protein complex has the structure of Formula IIIa or Formula IIIb: 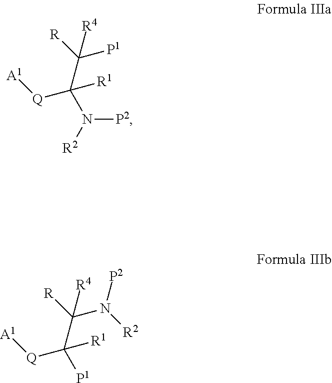
wherein A¹ is a monovalent organic moiety;
Q is 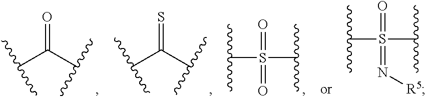
P¹ is A², and P² is hydrogen; or P¹ is hydroxyl, and P² is A²;
A² is the target protein;
R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl or —Si(R^{1a})₃; or
R and R¹ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl, and R⁴ is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; or
R and R² combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and R¹ and R⁴ are each, independently, hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a}); or
R and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, R¹ is hydrogen, halo, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, R is hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃; and R² is hydrogen, optionally substituted C₁-C₆ alkyl, optionally substituted C₁-C₆ acyl, —C(O)₂R^{2a}, —C(O)N(R^{2b})₂, —S(O)₂R^{2c}, —S(O)₂N(R^{2d})₂, —S(O)₂OR^{2e}, optionally substituted C₁-C₆ heteroalkyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; or
R¹ and R² combine to form an optionally substituted C₃-C₁₀ cycloalkyl, optionally substituted C₃-C₁₀ cycloalkenyl, optionally substituted C₈-C₁₀ cycloalkynyl, or optionally substituted 3- to 10-membered heterocycloalkyl, optionally substituted 5- to 10-membered heterocycloalkenyl, or optionally substituted 8- to 10-membered heterocycloalkynyl, and each of R and R⁴ is, independently, hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl or —Si(R^{1a})₃; or
R² and R⁴ combine to form an optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, or optionally substituted 8- to 10-membered heterocycloalkynyl, and each of R¹ and R is, independently, hydrogen, cyano, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₁-C₆ heteroalkenyl, optionally substituted C₁-C₆ heteroalkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, optionally substituted 5- to 10-membered heteroaryl, or —Si(R^{1a})₃;
wherein at least one of R and R⁴ is not hydrogen;
each R^{1a}, R^{2a}, R^{2c}, R^{2d}, and R^{2e} is, independently, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl;
R^{2b} is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted 5- to 10-membered heterocycloalkenyl, optionally substituted 8- to 10-membered heterocycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl; and
R⁵ is hydrogen, optionally substituted C₁-C₆ alkyl, optionally substituted C₂-C₆ alkenyl, optionally substituted C₂-C₆ alkynyl, optionally substituted C₃-C₁₀ cycloalkyl, optionally substituted C₃-C₁₀ cycloalkenyl, optionally substituted C₈-C₁₀ cycloalkynyl, optionally substituted 3- to 10-membered heterocycloalkyl, optionally substituted 5- to 10-membered heterocycloalkenyl, optionally substituted 8- to 10-membered heterocycloalkynyl, optionally substituted C₆-C₁₀ aryl, or optionally substituted 5- to 10-membered heteroaryl,
and wherein each hydrogen is independently, optionally, isotopically enriched for deuterium.

11. The compound of any one of claims 1 to 9, or a pharmaceutically acceptable salt thereof, or the compound/target protein complex of claim 10, wherein R is optionally substituted C₃-C₁₀ cycloalkyl, optionally substituted C₆-C₁₀ aryl, optionally substituted C₂-C₆ alkenyl, or optionally substituted C₂-C₆ alkynyl.

12. The compound or compound/target protein complex of claim 11, or a pharmaceutically acceptable salt thereof, wherein R is optionally substituted C₃-C₁₀ cycloalkyl.

13. The compound or compound/target protein complex of claim 12, or a pharmaceutically acceptable salt thereof, wherein R is optionally substituted cyclopropyl.

14. The compound or compound/target protein complex of claim 11, or a pharmaceutically acceptable salt thereof, wherein R is optionally substituted C₂-C₆ alkenyl, or optionally substituted C₂-C₆ alkynyl.

15. The compound or compound/target protein complex of any one of claims 1 to 14, or a pharmaceutically acceptable salt thereof, wherein R¹ is hydrogen.

16. The compound or compound/target protein complex of any one of claims 1 to 15, or a pharmaceutically acceptable salt thereof, wherein R² is hydrogen, optionally substituted C₁-C₆ alkyl, optionally substituted C₁-C₆ heteroalkyl, optionally substituted C₃-C₁₀ cycloalkyl, or optionally substituted 3- to 10-membered heterocycloalkyl.

17. The compound or compound/target protein complex of any one of claims 1 to 16, or a pharmaceutically acceptable salt thereof, wherein R⁴ is hydrogen.

18. The compound or compound/target protein complex of any one of claims 8 to 17, or a pharmaceutically acceptable salt thereof, wherein A¹ is a protein, a nucleic acid, or a small molecule.

19. The compound/target protein complex of any one of claims 10 to 18 wherein the target protein is a GTPase, GTPase activating protein, Guanine nucleotide-exchange factor, a heat shock protein, an ion channel, a coiled-coil protein, a kinase, a phosphatase, a ubiquitin ligase, a transcription factor, a chromatin modifier/remodeler, a protease, or a protein with classical protein-protein interaction domains and motifs.

20. A presenter protein/compound complex comprising a presenter protein and a compound of any one of claims 8, 9, and 11 to 18, or a pharmaceutically acceptable salt thereof.

21. A method of modulating a target protein, the method comprising contacting the target protein with the compound of any one of claims 8 to 18 or the presenter protein/compound complex claim 20.

22. A tri-complex comprising a presenter protein, a compound of any one of claims 8 to 18 or a pharmaceutically acceptable salt thereof, and a target protein.

23. A method of forming a tri-complex of claim 22, the method comprising contacting a target protein with a presenter protein/compound complex of claim 20.

24. A method of crosslinking a compound of any one of claims 8 to 18 to a second moiety, the method comprising contacting the moiety with the compound under conditions sufficient to form a covalent bond between the compound of any one of claims 8 to 18 and the second moiety.

25. A method of forming the presenter protein/compound complex of claim 20, the method comprising contacting a presenter protein with the compound of any one of claims 8 to 18 under conditions sufficient to permit the formation of a complex.

26. A method of forming the tri-complex of claim 22, the method comprising:
a) contacting a presenter protein with the compound of any one of claims 8 to 18 under conditions sufficient to permit the formation of presenter protein/compound complex; and
b) contacting the presenter protein/compound complex with a target protein under conditions that permit the formation of a tri-complex.

27. A method of treating a disease or disorder in a subject in need thereof, the method comprising administering to the subject a therapeutically effective amount of the compound of any one of claims 8 to 18.
